(19) United States
(12) Patent Application Publication
(10) Pub. No.: US 2022/0362386 A1
(43) Pub. Date: Nov. 17, 2022

(54) LIQUID COMPOSITIONS COMPRISING A LEVODOPA AMINO ACID CONJUGATE AND USES THEREOF

(71) Applicant: NeuroDerm, Ltd., Rehovot (IL)

(72) Inventors: Kenji Morokuma, Osaka (JP); Daisuke Iijima, Osaka (JP); Masataka Okuno, Osaka (JP); Akira Nakao, Osaka (JP); Liora Braiman-Wiksman, Rishon LeZion (IL); Elana Gazal, Rehovot (IL); Ronit Shaltiel-Karyo, Ramat HaSharon (IL); Alex Mainfeld, Petach Tikva (IL); Eduardo Zawoznik, Mazkeret Batya (IL); Shmuel Ben-Hamo, Kiriat Ata (IL)

(21) Appl. No.: 17/640,666

(22) PCT Filed: Sep. 3, 2020
(86) PCT No.: PCT/IL2020/050960

§ 371 Date: Mar. 4, 2022

Related U.S. Application Data
(60) Provisional application No. 62/896,518, filed on Sep. 5, 2019.
(60) Provisional application No. 62/897,648, filed on Sep. 9, 2019.

(51) Int. Cl. A61K 47/54; A61K 31/198; A61K 45/06

(52) U.S. Cl. CPC A61K 47/542; A61K 31/198; A61K 45/06

(57) ABSTRACT

Disclosed herein are liquid pharmaceutical formulations comprising levodopa amino acid conjugates that may further comprise a decarboxylase inhibitor, such as carbidopa, an antioxidant, a solvent, or any other pharmaceutically acceptable excipient. Further disclosed are methods of treating generative conditions and/or conditions characterized by reduced levels of dopamine in the brain, such as Parkinson's disease, comprising administering the disclosed liquid pharmaceutical formulations. Disclosed also are LDAA conjugate compounds.

### TECHNICAL FIELD

The present invention is directed to levodopa amino acids (LDAAs), salts thereof, compositions comprising the same, methods of preparing LDAAs, and methods of using the same in, for example, the treatment of conditions characterized by neurodegeneration and/or reduced levels of dopamine in the brain, e.g., Parkinson's disease.

### BACKGROUND

Parkinson's disease is a degenerative condition characterized by reduced concentration of the neurotransmitter dopamine in the brain. Levodopa (L-dopa or L-3,4-dihydroxyphenylalanine) is an immediate metabolic precursor of dopamine that, unlike dopamine, is able to cross the blood brain barrier, and is most commonly used for restoring the dopamine concentration in the brain. For the past 40 years, levodopa has remained the most effective therapy for the treatment of Parkinson's disease.

However, conventional treatments for Parkinson's disease with levodopa have proven to be inadequate for many reasons of record in the medical literature. For example, some patients eventually become less responsive to levodopa, such that previously effective doses eventually fail to produce any therapeutic benefit. Thus, the systemic administration of levodopa, while producing clinically beneficial effects at first, is complicated by the need to increase the doses to such high doses that may result in adverse side effects. For such reasons, the benefits of levodopa treatment often begin to diminish after about 3 or 4 years of therapy, irrespective of the initial therapeutic response.

The peripheral administration of levodopa is further complicated by the fact that only about 1-3% of the levodopa administered is able to enter the brain unaltered, wherein most of the levodopa is metabolized extracerebrally, predominantly by the decarboxylation of the levodopa to dopamine, which does not penetrate the blood brain barrier and therefore, is ineffective in treatment. The metabolic transformation of levodopa to dopamine is catalyzed by the aromatic L-amino acid decarboxylase enzyme, an ubiquitous enzyme with particularly high concentrations in the intestinal mucosa, liver, brain and brain capillaries. Due to the possibility of extracerebral metabolism of levodopa, it is necessary to administer large doses of levodopa, leading to high extracerebral concentrations of dopamine. The co-administration of levodopa and a peripheral dopamine decarboxylase (aromatic L-amino acid decarboxylase) inhibitor, such as carbidopa or benserazide, has been found to reduce the dosage requirements of levodopa and, respectively, some of the side effects; however, frequently, the obtained reduction is insufficient.

Finally, certain fluctuations in the clinical response to levodopa occur with increasing frequency with prolonged treatment. In some patients, these fluctuations relate to the timing of levodopa intake, known as “wearing-off reactions” or “end-of-dose akinesia”. In other instances, fluctuations in the clinical state are unrelated to the timing of doses and are generally referred to as “on-off phenomenon”. In the on-off phenomenon, “off-periods” of marked akinesia and bradykinesia alternate over the course of a few hours with “on-periods” of improved mobility, which are often associated with troublesome dyskinesia.

It is well accepted in the art that many of the disadvantages referred to above result from the unfavorable pharmacokinetic properties of levodopa and, more particularly, from its poor water solubility, bioavailability and fast degradation in vivo. Thus, there is still a need for effective therapeutic formulations for treating disorders such as Parkinson's disease.

Amino acids, which contain both amino and carboxylic groups, are the basic unit of proteins. Generally, amino acids are known to play a major role in the body, being involved in tissue protein formation and enzyme hormone formation. Therefore, any deficiency in amino acids affects protein synthesis. Amino acids are also known to regulate processes related to gene expression and further, amino acids modulate the protein function involved in messenger RNA translation. Several amino acids, such as tyrosine, are synthesized in the human body, while others, known as essential amino acids, such as arginine and lysine, are consumed by diet. The lanthionine amino acid is a natural, but non-proteinogenic, diamino diacid, and is structurally related to the amino acid cysteine. Lanthionine has a central monosulfur moiety bound to two alanine residues (R/S configuration), allowing the possibility of different stereomeric forms of lanthionine.

Amino acids are ionized in aqueous solutions, wherein the pH of the solution affects the ionic species of the amino acid and determines whether the amino acid will be in the form of a zwitterion, cation or anion. The permeability coefficients of the various compounds through the skin is dependent on their ionic form, wherein non-ionized species generally have higher permeability coefficients in comparison to ionized species and further, cations generally have higher permeability coefficients than anions.

U.S. Pat. Nos. 3,803,120, 4,035,507, 5,686,423 and US 2002/099013 disclose certain levodopa amino acid and levodopa peptide conjugates; however, details regarding formulations are not provided therein, and when provided, only solid oral formulations are contemplated. The theoretical option of preparing liquid compositions is briefly mentioned in U.S. Pat. No. 3,803,120 (U.S. '120, column 3, lines 49-53); however, no such compositions were prepared and moreover, it is erroneously disclosed that the conjugates are soluble (column 3, lines 65-66).

As detailed above, there is still a need to effective formulations, and particularly liquid formulations, for treating disorders such as Parkinson's disease.

### SUMMARY OF THE INVENTION

Provided herein, inter alia, are levodopa amino acids (LDAAs), salts thereof (e.g., pharmaceutically acceptable salts thereof), and compositions comprising the same (e.g., pharmaceutically acceptable compositions, for example, liquid pharmaceutical compositions). Also described herein are methods of preparing LDAAs, pharmaceutically acceptable salts thereof, and compositions comprising the same. Also disclosed are methods of using LDAAs, pharmaceutically acceptable salts thereof, and compositions comprising the same in, for example, the treatment of conditions characterized by neurodegeneration and/or reduced levels of dopamine in the brain, e.g., Parkinson's disease.

Disclosed herein is a liquid pharmaceutical composition comprising:

a levodopa amino acid conjugate (LDAA) of the general formula (I):

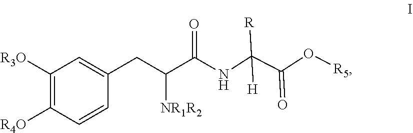

an enantiomer, diastereomer, racemate, ion, zwitterion, pharmaceutically acceptable salt thereof, or any combination thereof, wherein:

R is an amino acid side chain;

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl; and a pharmaceutically acceptable excipient.

In some embodiments, a liquid pharmaceutical composition described herein includes an LDAA of the general formula (I):

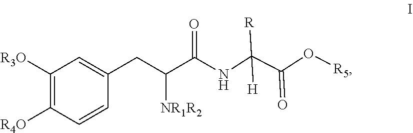

an enantiomer, diastereomer, racemate, ion, zwitterion, pharmaceutically acceptable salt thereof, or any combination thereof, where R is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and lanthionine side chains. For example, in embodiments described herein, R can be:

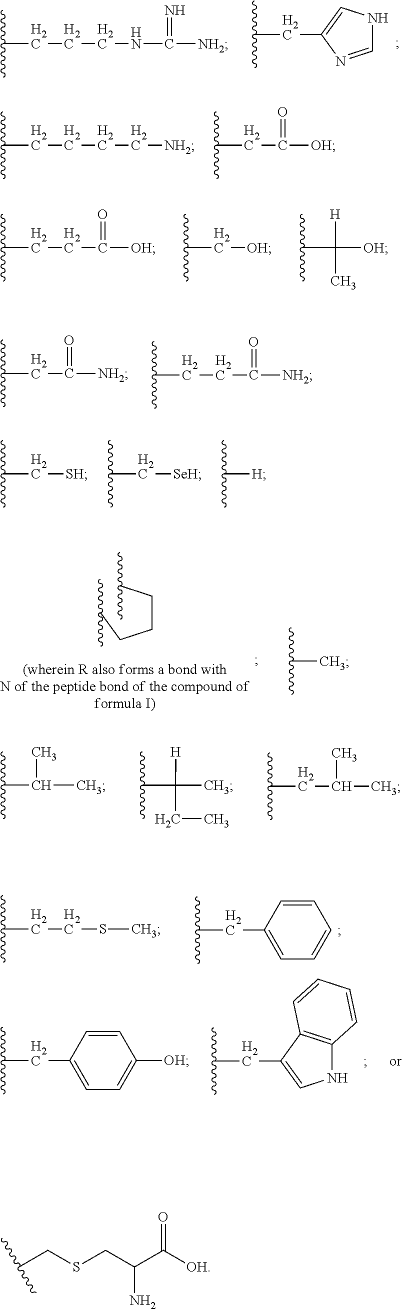

In some embodiments, a liquid pharmaceutical composition described herein includes an LDAA of the general formula (I), where R is an amino acid side chain selected from arginine, tyrosine or lysine. In some embodiments, R is the amino acid side chain of lanthionine-2.

Also disclosed herein is a liquid pharmaceutical composition that includes a LDAA of the general formula (I), an enantiomer, diastereomer, racemate, ion, zwitterion, pharmaceutically acceptable salt thereof, or any combination thereof, where each one of R₁, R₂, R₃, R₄ and R₅ are H. For example, in some embodiments, a liquid pharmaceutical composition described herein includes the compound:

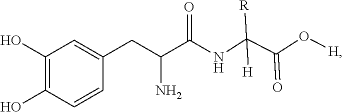

an enantiomer, diastereomer, racemate, ion, zwitterion, pharmaceutically acceptable salt thereof, or any combination thereof, where R is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and lanthionine side chains.

In some embodiments, a liquid pharmaceutical composition disclosed herein comprises between about 10 to about 45% w/v of one, two or more LDAA compounds, or an enantiomer, diastereomer, racemate, ion, zwitterion, pharmaceutically acceptable salt thereof, or any combination thereof.

In some embodiments, a liquid pharmaceutical composition disclosed herein has a pH in the range of between about 3 to about 10 at about 25° C.

In some embodiments, a liquid pharmaceutical composition disclosed herein can include a free base of the compound of formula I and a counterion.

In some embodiments, a liquid pharmaceutical composition disclosed herein can also include a decarboxylase inhibitor. For example, in some embodiments, the decarboxylase inhibitor is carbidopa. In some embodiments, a liquid pharmaceutical composition disclosed herein can include between about 0.25 to about 1.5% w/v of the decarboxylase inhibitor.

Any of the aforementioned liquid pharmaceutical compositions described herein can further include an antioxidant or a combination of two or more antioxidants. For example, in some embodiments, a liquid pharmaceutical composition described herein can include an antioxidant selected from the group consisting of ascorbic acid or a salt thereof, a cysteine, a bisulfite or a salt thereof, glutathione, a tyrosinase inhibitor, a Cu²⁺ chelator, and any combination thereof. In some embodiments, a liquid pharmaceutical composition described herein can include between about 0.05 to about 1.5% w/v of an antioxidant or a combination of antioxidants.

Any of the aforementioned liquid pharmaceutical composition described herein can further include at least one of: a catechol-O-methyltransferase (COMT) inhibitor, a monoamine oxidase (MAO) inhibitor, a surfactant, a buffer, an acid, a base, a solvent, or any combination thereof. For example, in some embodiments, a liquid pharmaceutical composition described herein can include a solvent, wherein the solvent may be N-methylpyrrolidone (NMP), tris(hydroxymethyl)aminomethane (tromethamine, TRIS), an ether such as tetrahydrofuran and 1,4-dioxane an amide, such as N,N-dimethylformamide and N-methylpyrrolidone, a nitrile, such as acetonitrile, a halogenated aliphatic hydrocarbon, such as chloroform and dichloromethane, an aromatic hydrocarbon, such as toluene or any combination thereof. It is noted that certain materials, such as tromethamine (TRIS) may be added to the composition and function, e.g., as a base, buffer, solvent, or any combination thereof. In some embodiments, a liquid pharmaceutical composition described herein can include a surfactant, where the surfactant is Tween-80. In some embodiments, a liquid pharmaceutical composition described herein can include a solvent and a surfactant, where the solvent is NMP and the surfactant is Tween-80. In some embodiments, the liquid pharmaceutical composition can include between about 0.1 to about 1.0% w/v of the surfactant, for example, 0.1 to about 1.0% w/v of Tween-80. In some embodiments, the liquid pharmaceutical composition can include between about 5.0 to about 40.0% w/v of the solvent, for example, between about 5.0 to about 40.0% w/v of NMP.

Also disclosed herein is a method of treating a neurodegenerative condition and/or a condition characterized by reduced levels of dopamine in the brain, wherein the method comprises administering a liquid pharmaceutical composition, wherein the liquid pharmaceutical composition comprises a levodopa amino acid conjugate (LDAA) of the general formula (I):

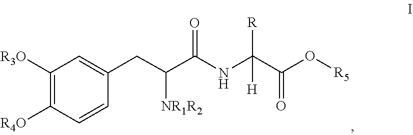

an enantiomer, diastereomer, racemate, ion, zwitterion, pharmaceutically acceptable salt thereof, or any combination thereof, wherein

R is an amino acid side chain;

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl; and

a pharmaceutically acceptable excipient.

For example, disclosed herein is a method of treating a neurodegenerative condition and/or a condition characterized by reduced levels of dopamine in the brain, wherein the method comprises administering a liquid pharmaceutical composition, wherein the liquid pharmaceutical composition comprises a LDAA of the general formula (I), an enantiomer, diastereomer, racemate, ion, zwitterion, pharmaceutically acceptable salt thereof, or any combination thereof, wherein R is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and lanthionine side chains. For example, in embodiments described herein, R can be:

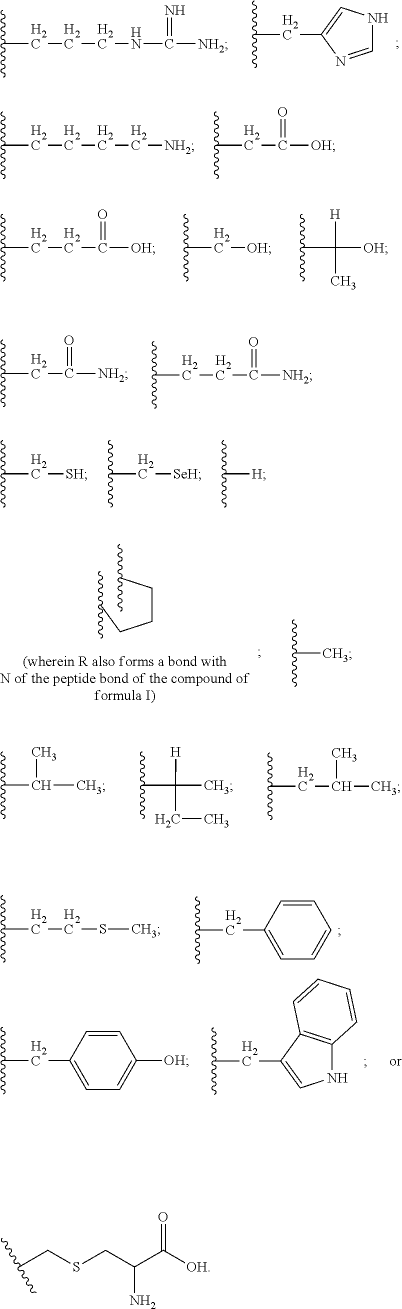

For example, disclosed herein is a method of treating a neurodegenerative condition and/or a condition characterized by reduced levels of dopamine in the brain, wherein the neurodegenerative condition is Parkinson's disease.

In some embodiments of the disclosed methods of treating, the liquid pharmaceutical composition is administered concomitantly with an additional active ingredient. For example, in some embodiments, the additional active ingredient is a decarboxylase inhibitor, a COMT inhibitor, a MAO inhibitor, or any combination thereof.

In some embodiments of the methods of treating disclosed herein, the liquid pharmaceutical composition is administered substantially continuously. In some embodiments, the liquid pharmaceutical composition is administered subcutaneously.

Also disclosed herein is a levodopa amino acid conjugate (LDAA) of the general formula (III):

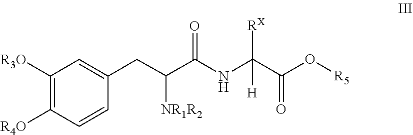

an enantiomer, diastereomer, racemate, ion, zwitterion, pharmaceutically acceptable salt thereof, or any combination thereof, wherein

R^{X} is an amino acid side chain; or an O-phosphorylated amino acid side chain thereof.

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl.

According to some embodiments, the amino acid side chain in R^{x} is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, ornithine, lanthionine and 3,4-dihydroxyphenylalanine side chain.

According to further embodiments, the amino acid side chain in R^{x} is selected from the group consisting of arginine, lysine, serine, glycine, alanine, valine, phenylalanine, tyrosine, ornithine, and 3,4-dihydroxyphenylalanine. According to some embodiments, each one of R₁, R₂ and R₅ are H; R₃, and R₄ independently is H or —P(═O)(OR′)₂; and R′ is H.

According to some embodiments, the levodopa amino acid conjugate (LDAA) selected from the group consisting of:
(2S)-2-amino-3-(3-hydroxy-4-phosphonooxyphenyl)propionamide,
2-[[(2S)-2-amino-3-(3-hydroxy-4-phosphonooxyphenyl)propanoyl]amino]ethanesulfonic acid,
(2S)-2-amino-6-[[(2S)-2-amino-3-(3-hydroxy-4-phosphonooxyphenyl)propanoyl]amino]hexanoic acid, and
(2S)-2-amino-5-[[(2S)-2-amino-3-(3-hydroxy-4-phosphonooxyphenyl)propanoyl]amino]pentanoic acid.

Embodiments of the invention are further directed to a method of treating Parkinson's disease in a patient in need thereof, comprising subcutaneously administering to the patient an effective amount of a compound as disclosed herein.

Also disclosed herein is a compound represented by:

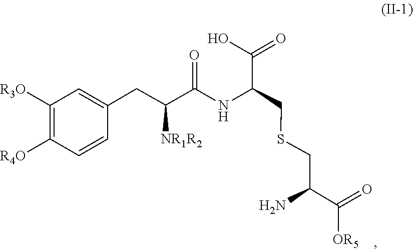

an enantiomer, diastereomer, racemate, ion, zwitterion, pharmaceutically acceptable salt thereof, or any combination thereof, wherein:

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl.

Also disclosed herein is a compound represented by:

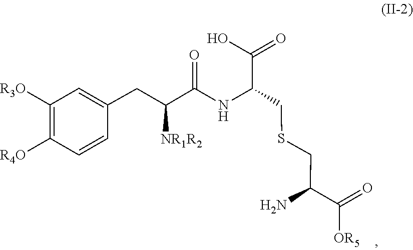

an enantiomer, diastereomer, racemate, ion, zwitterion, pharmaceutically acceptable salt thereof, or any combination thereof, wherein:

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl.

Disclosed herein is a compound of formula II-1 or II-2 wherein each one of R₁, R₂, R₃, R₄ and R₅ are H. For example, disclosed herein is the following compound:

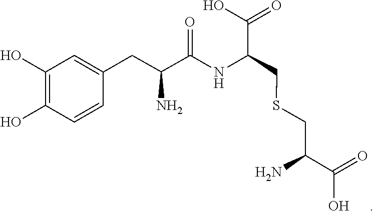 [SMILES: N[C@@H](CSC[C@@H](NC(=O)[C@@H](N)Cc1ccc(O)c(O)c1)C(=O)O)C(=O)O]

Also disclosed herein is the following compound:

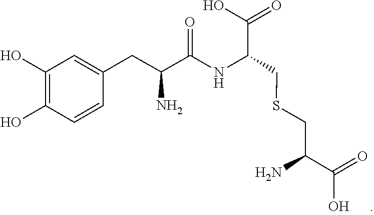 [SMILES: N[C@@H](CSC[C@H](NC(=O)[C@@H](N)Cc1ccc(O)c(O)c1)C(=O)O)C(=O)O]

Also disclosed herein is a process for preparing a liquid pharmaceutical composition, wherein said process comprises providing a pharmaceutically acceptable salt of a levodopa amino acid conjugate (LDAA) of formula (I):

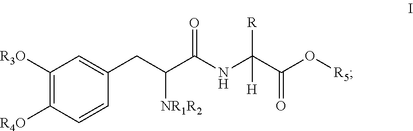

combining the pharmaceutically acceptable salt with at least one solvent thereby forming a solution, gel, cream, emulsion, or suspension; and

adjusting the pH of the solution, gel, cream, emulsion, or suspension, to a physiologically acceptable pH value, thereby providing the liquid pharmaceutical composition, wherein:

R is an amino acid side chain;

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl.

In some embodiments, a process for preparing a liquid pharmaceutical composition described herein includes providing a pharmaceutically acceptable salt of a LDAA of formula (I), an enantiomer, diastereomer, racemate, ion, zwitterion, pharmaceutically acceptable salt thereof, or any combination thereof, wherein R is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and lanthionine side chains. For example, in embodiments described herein, R can be:

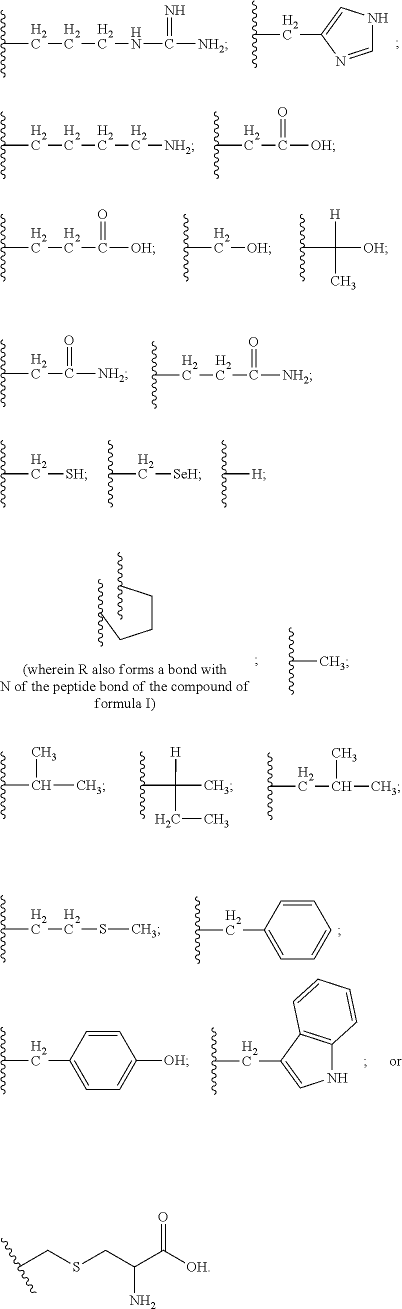

In some embodiments of a process described herein, the LDAA compound of Formula (I) in a pharmaceutically acceptable salt form is mixed with at least one solvent, thereby forming a solution. In some embodiments, the process includes a step of adjusting the pH that comprises adding a basic solution. For example, in some embodiments, the process includes a step of adjusting the pH that comprises adding a basic solution, and the basic solution comprises NaOH.

In some embodiments of a process described herein, the LDAA compound of Formula (I) is in a pharmaceutically acceptable solid salt form.

Also disclosed herein is a composition comprising a pharmaceutically acceptable salt of a compound represented by

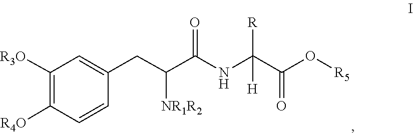

wherein:

R is an amino acid side chain;

R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;

R₃ and R₄ are each independently selected from the group consisting of H, (C₁-C₃)alkyl, C₃-C₆cycloalkyl, phenyl, and —P(═O)(OR′)₂;

R₅ is selected from the group consisting of H, (C₁-C₃)alkyl, C₃-C₆cycloalkyl and phenyl;

R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and

R″ is independently selected, in each occurrence, from the group consisting of a (C₁-C₆)alkyl, (C₂-C₆)alkenyl, and (C₂-C₆)alkynyl; and

a pharmaceutically acceptable excipient.

In some embodiments, a pharmaceutically acceptable salt disclosed herein is a pharmaceutically acceptable salt of the compound:

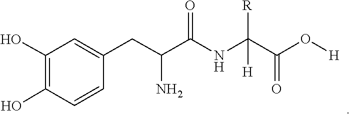

For example, disclosed herein is a composition that includes a pharmaceutically acceptable salt of a compound of formula (I), wherein the salt is a trifluoroacetic acid (TFA) salt.

Also disclosed herein are liquid pharmaceutical compositions comprising one or more of the following compounds:

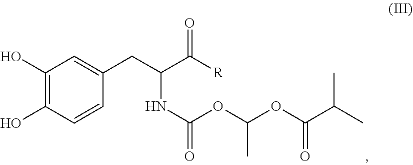

wherein R is H, a C₁-C₆alky, or an amino acid;

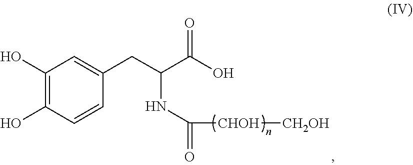

wherein n is 1, 2, 3, 4, or 5;

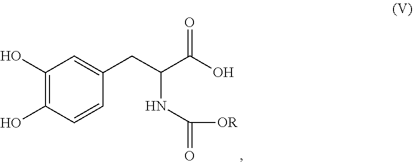

wherein R is H, a C₁-C₆alkyl, or an amino acid;

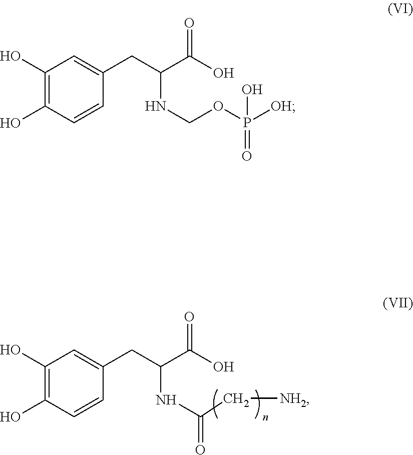

wherein n is 1, 2, 3, 4, or 5;

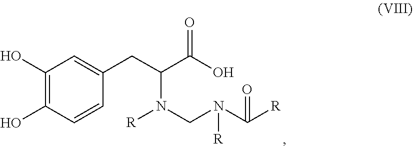

wherein R is H or a C₁-C_{6alkyl};

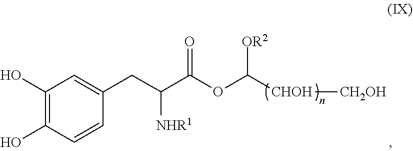

wherein R¹ is H or a C₁-C₆alkyl, wherein R² is H, a C₁-C₆alkyl, or an amino acid, and wherein n is 1, 2, 3, 4, or 5;

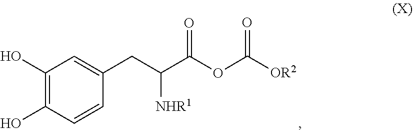

wherein R¹ is H or a C₁-C₆alkyl, wherein R² is H, a C₁-C₆alkyl, or an amino acid, and wherein n is 1, 2, 3, 4, or 5;

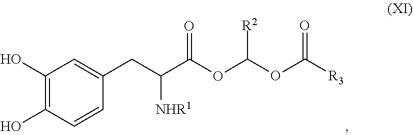

wherein R¹ is H or a C₁-C₆alkyl, wherein R² is an amino acid side chain, and wherein R³ is H or a C₁-C₆alkyl; or

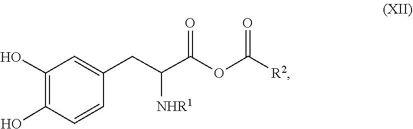

wherein R¹ is H or a C₁-C₆alkyl, and wherein R² is H or a C₁-C₆alkyl, and

a pharmaceutically acceptable excipient.

Also disclosed herein is a method of treating a neurodegenerative condition and/or a condition characterized by reduced levels of dopamine in the brain, wherein the method comprises administering a liquid pharmaceutical composition, wherein the liquid pharmaceutical composition comprises one or more of the following compounds:

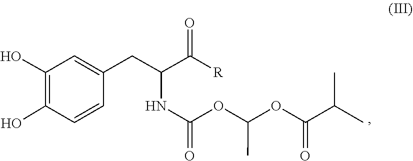

wherein R is H, a C₁-C₆alkyl, or an amino acid;

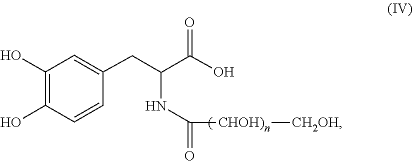

wherein n is 1, 2, 3, 4, or 5;

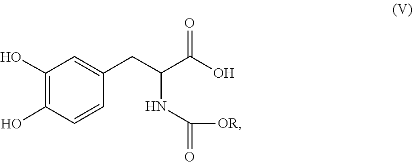

wherein R is H, a C₁-C₆alkyl, or an amino acid;

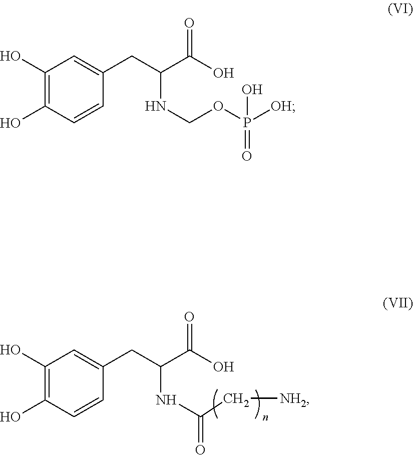

wherein n is 1, 2, 3, 4, or 5;

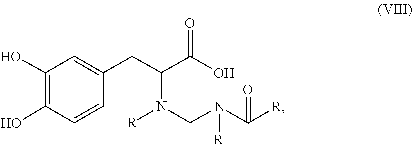

wherein R is H or a C₁-C₆alkyl;

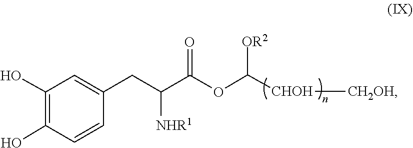

wherein R¹ is H or a C₁-C₆alkyl, wherein R² is H, a C₁-C₆alkyl, or an amino acid, and wherein n is 1, 2, 3, 4, or 5;

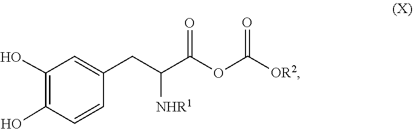

wherein R¹ is H or a C₁-C₆alkyl, wherein R² is H, a C₁-C₆alkyl, or an amino acid, and wherein n is 1, 2, 3, 4, or 5;

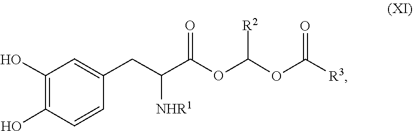

wherein R¹ is H or a C₁-C₆alkyl, wherein R² is an amino acid side chain, and wherein R³ is H or a C₁-C₆alkyl; or

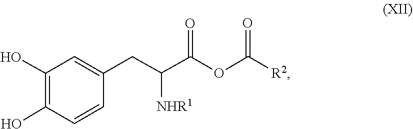

wherein R¹ is H or a C₁-C₆alkyl, and wherein R² is H or a C₁-C₆alkyl, and

a pharmaceutically acceptable excipient.

Also disclosed herein is a process for preparing a liquid pharmaceutical composition, wherein said process comprises:

providing a pharmaceutically acceptable salt of one of the following compounds:

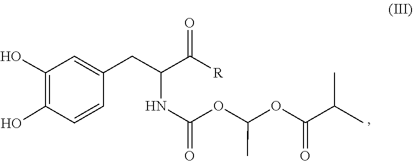

wherein R is H, a C₁-C₆alkyl, or an amino acid;

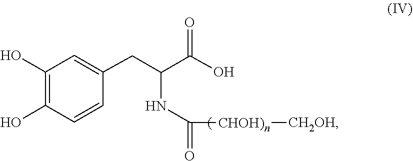

wherein n is 1, 2, 3, 4, or 5;

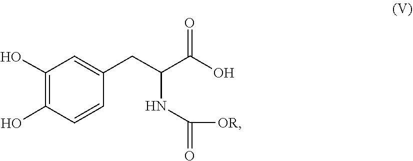

wherein R is H, a C₁-C₆alkyl, or an amino acid;

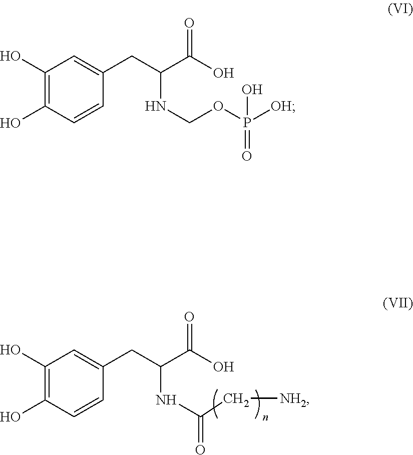

wherein n is 1, 2, 3, 4, or 5;

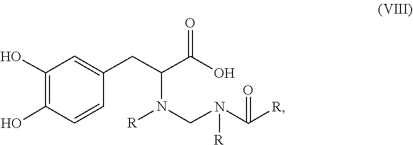

wherein R is H or a C₁-C₆alkyl;

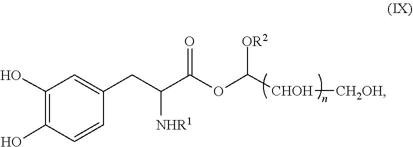

wherein R¹ is H or a C₁-C₆alkyl, wherein R² is H, a C₁-C₆alkyl, or an amino acid, and wherein n is 1, 2, 3, 4, or 5;

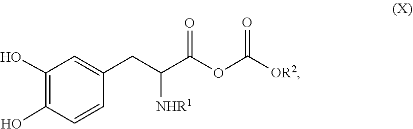

wherein R¹ is H or a C₁-C₆alkyl, wherein R² is H, a C₁-C₆alkyl, or an amino acid, and wherein n is 1, 2, 3, 4, or 5;

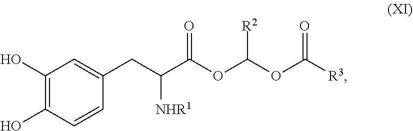

wherein R¹ is H or a C₁-C₆alkyl, wherein R² is an amino acid side chain, and wherein R³ is H or a C₁-C₆alkyl; or

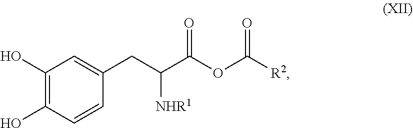

wherein R¹ is H or a C₁-C₆alkyl, and wherein R² is H or a C₁-C₆alkyl

combining the pharmaceutically acceptable salt with at least one solvent thereby forming a solution, gel, cream, emulsion, or suspension; and

adjusting the pH of the solution, gel, cream, emulsion, or suspension, to a physiologically acceptable pH value, thereby providing the liquid pharmaceutical composition.

### BRIEF DESCRIPTION OF THE DRAWINGS

FIG. 1 depicts the remaining percentage of various levodopa amino acid (LDAA) compounds in the TFA salt form, following human liver microsomes metabolism, tested at 0, 15, 30, 45 and 60 minutes;

FIG. 2 presents Table 26, which includes the pharmacokinetic parameters derived from a subcutaneous bolus study performed on Göttingen minipigs;

FIG. 3 is a graph presenting the LDAA compound concentration as a factor of time, following the subcutaneous bolus administration of 5 mg/Kg of each tested LDAA compound to Göttingen minipigs;

FIG. 4 is a graph presenting the levodopa concentration as a factor of time, following the subcutaneous bolus administration of 5 mg/Kg of each tested LDAA compound to minipigs;

FIG. 5 is a graph presenting the LD-Tyr free base and levodopa concentrations as a factor of time, during and following a continuous subcutaneous administration of an LD-Tyr free base solution to Göttingen minipigs for 24 hours;

FIG. 6A presents a histopathology image obtained two weeks after recovery from a 24 hour continuous subcutaneous administration of an LD-Tyr free base solution to Göttingen minipigs;

FIG. 6B presents a histopathology image obtained two weeks after recovery from a 24 hour continuous subcutaneous administration of the vehicle of the LD-Tyr free base solution (without the LD-Tyr free base itself) to Göttingen minipigs;

FIG. 6C presents a histopathology image obtained after 24 hours of having a sham (needle alone) inserted into Göttingen minipigs; and

FIG. 7 presents the incidence of subcutaneous inflammation in Göttingen minipigs after two weeks of recovery from a 24 hour of administration of the LD-Tyr free base solution, the solution vehicle and the sham.

### DETAILED DESCRIPTION OF THE INVENTION

The features and other details of the disclosure will now be more particularly described. Certain terms employed in the specification, examples, and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The terms “treat,” “treatment,” “treating,” and the like are used herein to generally refer to obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refer to any and all solvents, dispersion media, coatings, isotonic and absorption delaying agents, and the like, that are compatible with pharmaceutical administration.

The terms “pharmaceutical composition” and “pharmaceutical formulation” as used herein refer to a composition or formulation comprising at least one biologically active compound, for example, a levodopa amino acid conjugate, or a pharmaceutically acceptable salt thereof, as disclosed herein, formulated together with one or more pharmaceutically acceptable excipients.

The term “pharmaceutically acceptable salt(s)” as used herein refers to salts of acidic or basic groups that may be formed with the conjugates used in the compositions disclosed herein.

“Individual,” “patient,” or “subject” are used interchangeably and include any animal, including mammals, mice, rats, other rodents, rabbits, dogs, cats, swine, cattle, sheep, horses, or non-human primates, and humans. In some embodiments, the mammal treated in the methods of the invention is a human suffering from neurodegenerative condition, such as Parkinson's disease.

The term “about”, as used herein, unless specifically mentioned otherwise, or unless a person skilled in the art would have understood otherwise, is considered to cover a range of ±10% of the listed value(s). It is further noted that any value provided may also be considered to cover a range of ±10% of that value, even without the use of the term “about”. This includes the values in the examples section, which may vary according to the utensils and machinery used, the purity of the compounds, etc.

The terms “stable” or “stable overnight”, as used herein, unless specifically mentioned otherwise, or unless a person skilled in the art would have understood otherwise, refer to a substance that was physically stable for at least 12 hours, such that, upon visual view of the substance, e.g., formulation, under magnification of at least ×1.75, no precipitants were visible.

The term “liquid” as used herein, unless specifically mentioned otherwise, or unless a person skilled in the art would have understood otherwise, refers to any type of fluid, including gels, aqueous and non-aqueous compositions, and the like.

The term “concomitant” as used herein, unless specifically mentioned otherwise, or unless a person skilled in the art would have understood otherwise, refers to any type of combined administration of two or more active ingredients, including administration of those active ingredients at the same time, either in separate or the same composition, as well as administering the two or more active ingredients sequentially, consecutively, on the same day, with a predefined period of time separating the administration of the active ingredients from one another, and the like.

The terms “continuously” and “substantially continuously” as used herein, unless specifically mentioned otherwise, or unless a person skilled in the art would have understood otherwise, refer to a period of time during which a composition is administered over the entire period of time, with intermissions of less than about 24 hours, about 12 hours, about five hours, about three hours, about one hour, about 30 minutes, about 15 minutes, about five minutes or about one minute. The period of time during which a composition is administered may be at least about six hours, about eight hours, about 12 hours, about 15 hours, about 18 hours, about 21 hours, about 24 hours, three days, seven days, two weeks, a month, three months, six months, a year, two years, three years, five years, ten years, etc.

The term “physiologically acceptable pH value” and the like, as used herein, unless specifically mentioned otherwise, or unless a person skilled in the art would have understood otherwise, refers to pH values in the range of between about 4.5 to about 10. It is further noted that when pH values are provided, including in the examples, the values may be in the range of about ±0.1 and/or ±10% of the listed value(s), such that if the measured pH is 8.1, the same formulation may be prepared to provide a pH of about 8.0 or 8.2. Such differences may be due to temperature changes, various measuring devices, etc.

Embodiments of the invention are directed to a liquid pharmaceutical composition comprising a levodopa amino acid conjugate (LDAA) of the general formula (I):

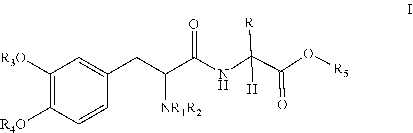

an enantiomer, diastereomer, racemate, ion, zwitterion, pharmaceutically acceptable salt thereof, or any combination thereof, wherein:

R is an amino acid side chain;

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

According to some embodiments, R is an amino acid side chain of any natural, synthetic, non-natural, or non-proteogenic amino acid, for example, the side chain of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, lanthionine, selenocysteine, pyrrolysine, ADDA amino acid ((2S,3S,4E,6E,8S,9S)-3-Amino-9-methoxy-2,6,8-trimethyl-10-phenyldeca-4,6-dienoic acid), beta-alanine, 4-Aminobenzoic acid, gamma-aminobutyric acid, S-aminoethyl-L-cysteine, 2-aminoisobutyric acid, aminolevulinic acid, azetidine-2-carboxylic acid, canaline, canavanine, carboxyglutamic acid, chloroalanine, citrulline, cystine, dehydroalanine, diaminopimelic acid, dihydroxyphenylglycine, enduracididine, homocysteine, homoserine, 4-hydroxyphenylglycine, hydroxyproline, hypusine, beta-leucine, norleucine, norvaline, ornithine, penicillamine, plakohypaphorine, pyroglutamic acid, quisqualic acid, sarcosine, theanine, tranexamic acid, tricholomic acid, or any isomer thereof. In this respect it is noted that R may be either of the known isomers of lanthionine, wherein one is referred to herein as lanthionine-1 or lanthionine-peak 1, while the other is referred to herein as lanthionine-2 or lanthionine-peak 2. Further, the levodopa lanthionine conjugates may be referred to herein as LD-LA, LD-LA 1 (for the first isomer), LD-LA 2 (for the second isomer), LD-lanthionine 1 (for the first isomer), LD-lanthionine 2 (for the second isomer), and the like.

According to some embodiments, R is an amino acid side chain of arginine, tyrosine, lysine, aspartic acid, asparagine, tryptophan, glutamine, glutamic acid, glycine, or lanthionine. According to some embodiments, R is an amino acid side chain of arginine, tyrosine, lysine, lanthionine-2, tryptophan, glutamic acid or glycine. According to some embodiments, R is an amino acid side chain of arginine, tyrosine, lysine or lanthionine-2. According to some embodiments, R is an amino acid side chain of arginine, tyrosine or lysine. According to some embodiments, R is the amino acid side chain of lanthionine-2.

According to some embodiments, each one of R₁, R₂, R₃, R₄ and R₅ are H. According to some embodiments, R″ has at least 10 carbon atoms. According to some embodiments, the liquid pharmaceutical composition comprises a mixture of two or more LDAA compounds.

According to some embodiments, the liquid pharmaceutical composition comprises an LDAA compound in a pharmaceutically acceptable salt form. According to some embodiments, the LDAA salt is selected from a trifluoroacetic acid (TFA) salt, an HCl salt, fumaric acid salt, lactate salt, maleic acid salt, gluceptic acid salt, phosphoric acid salt, sulfuric acid salt, HBr salt, nitric acid salt, acetic acid salt, propionic acid salt, hexanoic acid salt, cyclopentanepropionic acid salt, glycolic acid salt, pyruvic acid salt, lactic acid salt, hippuric acid salt, methanesulfonic acid salt, ascorbic acid salt, malonic acid salt, oxalic acid salt, maleic acid salt, tartaric acid salt, citric acid salt, succinic acid salt, benzoic acid salt, cinnamic acid salt, a sulfonic acid salt, lauryl sulfuric acid salt, gluconic acid salt, glutamic acid salt, hydroxynaphthoic acid salt, salicylic acid salt, stearic acid salt, muconic acid salt, an alkali metal salt, such as lithium salt, sodium salt or potassium salt, an alkaline earth metal salt, such as calcium salt or magnesium salt, an aluminum salt, an ethanolamine salt, diethanolamine salt, triethanolamine salt, N-methylglucamine salt, dicyclohexylamine salt, adipate salt, alginate salt, ascorbate salt, aspartate salt, benzenesulfonate salt, bisulfate salt, borate salt, butyrate salt, camphorate butyrate salt, camphorsulfonate butyrate salt, digluconate butyrate salt, dodecylsulfate butyrate salt, ethanesulfonate butyrate salt, glucoheptonate butyrate salt, glycerophosphate butyrate salt, gluconate butyrate salt, hemisulfate butyrate salt, heptanoate butyrate salt, hydroiodide butyrate salt, 2-hydroxy-ethanesulfonate butyrate salt, lactobionate butyrate salt, laurate butyrate salt, methanesulfonate butyrate salt, 2-naphthalenesulfonate butyrate salt, nicotinate butyrate salt, oleate butyrate salt, palmitate butyrate salt, pamoate butyrate salt, pectinate butyrate salt, persulfate butyrate salt, 3-phenylpropionate butyrate salt, phosphate butyrate salt, picrate butyrate salt, pivalate butyrate salt, tartrate butyrate salt, thiocyanate butyrate salt, p-toluenesulfonate butyrate salt, undecanoate butyrate salt, valerate salts, or any combination thereof.

The liquid pharmaceutical composition of the invention may comprise between about 2.5 to about 70% w/v of an LDAA compound, an enantiomer, diastereomer, racemate, ion, zwitterion, pharmaceutically acceptable salt thereof, or any combination thereof, or any combination of two or more LDAA compounds, enantiomers, diastereomers, racemates, ions, zwitterions, pharmaceutically acceptable salts thereof, or any combination thereof. According to some embodiments, the liquid pharmaceutical composition comprises between about 2.5 to about 5% w/v, between about 5 to about 10% w/v, between about 10 to about 15% w/v, between about 15 to about 20% w/v, between about 20 to about 25% w/v, between about 25 to about 30% w/v, between about 30 to about 35% w/v, between about 35 to about 40% w/v, between about 40 to about 45% w/v, between about 45 to about 50% w/v, between about 50 to about 55% w/v, between about 55 to about 60% w/v, between about 60 to about 65% w/v, between about 65 to about 70% w/v, between about 10 to about 12.5% w/v, between about 12.5 to about 17.5% w/v, between about 17.5 to about 22.5% w/v, between about 22.5 to about 27.5% w/v, between about 27.5 to about 32.5% w/v, between about 32.5 to about 37.5% w/v, between about 37.5 to about 42.5% w/v, between about 42.5 to about 45% w/v, about 10% w/v, about 12.5% w/v, about 15% w/v, about 17.5% w/v, about 20% w/v, about 22.5% w/v, about 25% w/v, about 27.5% w/v, about 30% w/v, about 32.5% w/v, about 35% w/v, about 37.5% w/v, about 40% w/v, about 42.5% w/v, about 45% w/v, about 47.5% w/v, about 50% w/v, about 52.5% w/v, about 55% w/v, about 57.5% w/v, about 60% w/v, about 62.5% w/v, about 65% w/v, about 67.5% w/v, about 70% w/v of an LDAA compound, an enantiomer, diastereomer, racemate, ion, zwitterion, pharmaceutically acceptable salt thereof, or any combination thereof, or any combination of two or more LDAA compounds, enantiomers, diastereomers, racemates, ions, zwitterions, pharmaceutically acceptable salts thereof, or any combination thereof.

The pH of the liquid pharmaceutical composition of the invention may be between about 4.5 to about 10 at about 25° C. According to some embodiments, the pH of the liquid pharmaceutical compositions is between about 4.5 to about 5 at about 25° C. According to some embodiments, the pH of the liquid pharmaceutical compositions is between about 5 to about 6 at about 25° C. According to some embodiments, the pH of the liquid pharmaceutical compositions is between about 6 to about 7 at about 25° C. According to some embodiments, the pH of the liquid pharmaceutical compositions is between about 7 to about 8 at about 25° C. According to some embodiments, the pH of the liquid pharmaceutical compositions is between about 8 to about 9 at about 25° C. According to some embodiments, the pH of the liquid pharmaceutical compositions is between about 9 to about 10 at about 25° C. According to some embodiments, the pH of the liquid pharmaceutical compositions is between about 4.5 to about 5.5 at about 25° C. According to some embodiments, the pH of the liquid pharmaceutical compositions is between about 5.5 to about 6.5 at about 25° C. According to some embodiments, the pH of the liquid pharmaceutical compositions is between about 6.5 to about 7.5 at about 25° C. According to some embodiments, the pH of the liquid pharmaceutical compositions is between about 7.5 to about 8.5 at about 25° C. According to some embodiments, the pH of the liquid pharmaceutical compositions is between about 8.5 to about 9.5 at about 25° C. According to some embodiments, the pH of the liquid pharmaceutical compositions is between about 9.5 to about 10 at about 25° C.

According to some embodiments, the liquid pharmaceutical composition further comprises a decarboxylase inhibitor. According to some embodiments, the decarboxylase inhibitor is selected from carbidopa, benserazide, methyldopa, 3′,4′,5,7-Tetrahydroxy-8-methoxyisoflavone, alpha-difluoromethyl-dopa, or any combination thereof. According to some embodiments, the decarboxylase inhibitor is carbidopa.

The liquid pharmaceutical composition of the invention may comprise between about 0.25 to about 3.0% w/v of a decarboxylase inhibitor, e.g., carbidopa. According to some embodiments, the liquid pharmaceutical compositions comprises between about 0.25 to about 0.5% w/v, between about 0.5 to about 0.75% w/v, between about 0.75 to about 1.0% w/v, between about 1.0 to about 1.25% w/v, between about 1.25 to about 1.5% w/v, between about 1.5 to about 1.75% w/v, between about 1.75 to about 2.0% w/v, between about 2.0 to about 2.25% w/v, between about 2.25 to about 2.5% w/v, between about 2.5 to about 2.75% w/v, between about 2.75 to about 3.0% w/v, between about 0.5 to about 1.0% w/v, between about 0.6 to about 0.9% w/v, between about 0.7 to about 0.8% w/v, about 0.5% w/v, about 0.55% w/v, about 0.6% w/v, about 0.65% w/v, about 0.7% w/v, about 0.75% w/v, about 0.8% w/v, about 0.85% w/v, of a decarboxylase inhibitor, such as carbidopa.

According to some embodiments, the liquid pharmaceutical composition further comprises a buffer. According to some embodiments, the buffer is selected from citrate buffer, citric acid buffer, sodium acetate buffer, acetic acid buffer, tartaric acid buffer, phosphate buffer, succinic acid buffer, Tris buffer, glycine buffer, hydrochloric acid buffer, potassium hydrogen phthalate buffer, sodium buffer, sodium citrate tartrate buffer, sodium hydroxide buffer, sodium dihydrogen phosphate buffer, disodium hydrogen phosphate buffer, tromethamine (TRIS), or any combination thereof. The liquid pharmaceutical compositions may comprise between about 0.1 to about 30.0% w/v of a buffer. According to some embodiments, the liquid pharmaceutical composition comprises between about 0.1 to about 1.0% w/v, between about 1.0 to about 2.0% w/v, between about 2.0 to about 3.0% w/v, between about 3.0 to about 4.0% w/v, between about 4.0 to about 5.0% w/v, between about 5.0 to about 6.0% w/v, between about 6.0 to about 7.0% w/v, between about 8.0 to about 9.0% w/v, between about 9.0 to about 10.0% w/v, between about 10.0 to about 15.0% w/v, between about 15.0 to about 20.0% w/v, between about 20.0 to about 25.0% w/v, between about 25.0 to about 30.0% w/v of a buffer.

According to some embodiments, the liquid pharmaceutical compositions further comprises an acid or a base, e.g., in order to provide a composition with a pre-defined pH. According to some embodiments, the acid is selected from HCl, HBr, methanesulfonic acid, ascorbic acid, acetic acid, citric acid, or any combination thereof. According to some embodiments, the base is selected from NaOH, Ca(OH)₂, ammonium hydroxide, arginine, magnesium hydroxide, potassium hydroxide, meglumine, tromethamine (TRIS), triethylamine, diisopropylethylamine, diazabicycloundecene or any combination thereof. The liquid pharmaceutical compositions may comprise between about 0.1 to about 30.0% w/v of a base or acid. According to some embodiments, the liquid pharmaceutical composition comprises between about 0.1 to about 1.0% w/v, between about 1.0 to about 2.0% w/v, between about 2.0 to about 3.0% w/v, between about 3.0 to about 4.0% w/v, between about 4.0 to about 5.0% w/v, between about 5.0 to about 6.0% w/v, between about 6.0 to about 7.0% w/v, between about 8.0 to about 9.0% w/v, between about 9.0 to about 10.0, between about 10.0 to about 11.0, between about 11.0 to about 12.0, between about 12.0 to about 13.0, between about 13.0 to about 14.0, between about 14.0 to about 15.0, between about 15.0 to about 16.0, between about 16.0 to about 17.0, between about 17.0 to about 18.0, between about 18.0 to about 19.0, between about 19.0 to about 20.0, between about 20.0 to about 21.0, between about 21.0 to about 22.0, between about 22.0 to about 23.0, between about 23.0 to about 24.0, between about 24.0 to about 25.0, between about 25.0 to about 26.0, between about 26.0 to about 27.0, between about 27.0 to about 28.0, between about 28.0 to about 29.0, between about 29.0 to about 30.0, of a base or acid.

According to some embodiments, the liquid pharmaceutical composition further comprises an antioxidant. According to some embodiments, the antioxidant is selected from ascorbic acid or a salt thereof, a cysteine, a bisulfite or a salt thereof, glutathione, a tyrosinase inhibitor, a bivalent cation, such as a Cu⁺² chelator, butylated hydroxy toluene (BHT), beta hydroxy acid (BHA) tocopherol, gentisic acid, tocopherol, tocopherol derivative, such as tocopherol acetate or tocopherol succinate, thioglycerol, or any combination thereof.

According to some embodiments, the antioxidant is an ascorbic acid salt selected from sodium ascorbate, calcium ascorbate, potassium ascorbate, or any combination thereof. According to some embodiments, the antioxidant is a cysteine selected from L-cysteine, N-acetyl cysteine (NAC) or any combination thereof. According to some embodiments, the antioxidant is the bisulfite salt sodium metabisulfite. According to some embodiments, the antioxidant is the tyrosinase inhibitor captopril. According to some embodiments, the antioxidant is a Cu⁺² chelator is selected from Na₂-EDTA and Na₂-EDTA-Ca, or any combination thereof.

According to some embodiments, the antioxidant is selected from methimazole, quercetin, arbutin, aloesin, N-acetylglucoseamine, retinoic acid, alpha-tocopheryl ferulate, Mg ascorbyl phosphate (MAP), substrate analogues, such as sodium benzoate, L-phenylalanine, dimercaptosuccinic acid, D-penicillamine, trientine-HCl, dimercaprol, clioquinol, sodium thiosulfate, triethylenetetramine, tetraethylenepentamine, curcumin, neocuproine, tannin, cuprizone, sulfite salts, such as sodium hydrogen sulfite or sodium metabisulfite, lipoic acid, CB4 (N-acetyl CysGlyProCys amide), CB3 (N-acetyl CysProCys amide), AD4 (N-acetyl cysteine amide), AD6 (N-acetylGluCysGly amide), AD7 (N-acetylCysGly amide), vitamin E, di-tert-butyl methyl phenol, tert-butyl-methoxyphenol, a polyphenol, a tocopherol, an ubiquinone, caffeic acid, or any combination thereof.

The liquid pharmaceutical compositions of the invention may comprise between about 0.05 to about 2.0% w/v of an antioxidant or a combination of antioxidants. According to some embodiments, the liquid pharmaceutical composition comprises between about 0.05 to about 0.1% w/v, about 0.1 to about 0.2% w/v, about 0.2 to about 0.3% w/v, about 0.3 to about 0.4% w/v, about 0.4 to about 0.5% w/v, about 0.5 to about 0.6% w/v, about 0.7 to about 0.8% w/v, about 0.8 to about 0.9% w/v, about 0.9 to about 1.0% w/v, about 1.0 to about 1.1% w/v, about 1.1 to about 1.2% w/v, about 1.2 to about 1.3% w/v, about 1.3 to about 1.4% w/v, about 1.4 to about 1.5% w/v, about 1.5 to about 1.6% w/v, about 1.6 to about 1.7% w/v, about 1.7 to about 1.8% w/v, about 1.8 to about 1.9% w/v, about 1.9 to about 2.0% w/v, about 0.75% w/v, about 0.8% w/v, about 0.85% w/v, about 0.9% w/v, about 0.95% w/v, about 1.0% w/v, about 1.05% w/v, about 1.1% w/v, about 1.15% w/v, about 1.2% w/v, of an antioxidant or a combination of antioxidants.

According to some embodiments, the liquid pharmaceutical composition further comprises a catechol-O-methyltransferase (COMT) inhibitor. According to some embodiments, the COMT inhibitor is selected from entacapone, tolcapone, opicapone or any combination thereof. According to some embodiments, the liquid pharmaceutical composition comprises between about 0.1 to about 5.0% w/v of a COMT inhibitor. According to some embodiments, the liquid pharmaceutical composition comprises between about 0.1 to about 1.0% w/v of a COMT inhibitor. According to some embodiments, the liquid pharmaceutical composition comprises between about 1.0 to about 2.0% w/v of a COMT inhibitor. According to some embodiments, the liquid pharmaceutical composition comprises between about 2.0 to about 3.0% w/v of a COMT inhibitor. According to some embodiments, the liquid pharmaceutical composition comprises between about 3.0 to about 4.0% w/v of a COMT inhibitor. According to some embodiments, the liquid pharmaceutical composition comprises between about 4.0 to about 5.0% w/v of a COMT inhibitor. According to some embodiments, the liquid pharmaceutical composition may be administered concomitantly with a COMT inhibitor.

According to some embodiments, the liquid pharmaceutical composition further comprises a monoamine oxidase (MAO) inhibitor. The MAO inhibitor may be a MAO-A inhibitor or a MAO-B inhibitor. According to some embodiments, the liquid pharmaceutical composition comprises between about 0.1 to about 5.0% w/v of a MAO inhibitor. According to some embodiments, the liquid pharmaceutical composition comprises between about 0.1 to about 1.0% w/v of a MAO inhibitor. According to some embodiments, the liquid pharmaceutical composition comprises between about 1.0 to about 2.0% w/v of a MAO inhibitor. According to some embodiments, the liquid pharmaceutical composition comprises between about 2.0 to about 3.0% w/v of a MAO inhibitor. According to some embodiments, the liquid pharmaceutical composition comprises between about 3.0 to about 4.0% w/v of a MAO inhibitor. According to some embodiments, the liquid pharmaceutical composition comprises between about 4.0 to about 5.0% w/v of a MAO inhibitor. According to some embodiments, the MAO inhibitor is selected from moclobemide, rasagiline, selegiline, safinamide, or any combination thereof. According to some embodiments, the liquid pharmaceutical composition may be administered concomitantly with a MAO inhibitor.

According to some embodiments, the liquid pharmaceutical composition further comprises a surfactant. According to some embodiments, the surfactant is selected from Tween-80, Tween-60, Tween-40, Tween-20, Tween-65, Tween-85, Span 20, Span 40, Span 60, Span 80, Span 85, polyoxyl 35 castor oil (Cremophor EL), polyoxyethylene-660-hydroxystearate (macrogol 660), or Poloxamer 188 (Pluronic® F-68), or any combination thereof. The liquid pharmaceutical composition of the invention may include between about 0.1 to about 3.0% w/v of a surfactant or combination of two or more surfactants. According to some embodiments, the liquid pharmaceutical composition comprises between about 0.1 to about 0.2% w/v, between about 0.2 to about 0.3% w/v, between about 0.3 to about 0.4% w/v, between about 0.4 to about 0.5% w/v, between about 0.5 to about 0.6% w/v, between about 0.6 to about 0.7% w/v, between about 0.7 to about 0.8% w/v, between about 0.8 to about 0.9% w/v, between about 0.9 to about 1.0% w/v, between about 1.0 to about 1.5% w/v, between about 1.5 to about 2.0% w/v, between about 2.0 to about 2.5% w/v, between about 2.5 to about 3.0% w/v of a surfactant or combination of two or more surfactants.

The liquid pharmaceutical composition may further comprise an additional pharmaceutically acceptable excipient, such as N-methylpyrrolidone (NMP), polyvinylpyrrolidone (PVP), propylene glycol, a preservative, a pharmaceutically acceptable vehicle, a stabilizer, a dispersing agent, a suspending agent, an amino sugar, a calcium chelator, protease inhibitors, or any combination thereof. The liquid pharmaceutical composition of the invention may comprise between about 5.0 to about 80.0% w/v or an additional pharmaceutically acceptable excipient, e.g., a solvent, such as NMP or a buffer or any other co-solvent.

According to some embodiments, the liquid pharmaceutical composition of the invention comprises between about 5.0 to about 10.0% w/v, between about 10.0 to about 15.0% w/v, between about 15.0 to about 20.0% w/v, between about 20.0 to about 25.0% w/v, between about 25.0 to about 30.0% w/v, between about 30.0 to about 35.0% w/v, between about 35.0 to about 40.0% w/v, between about 40.0 to about 45.0% w/v, between about 45.0 to about 50.0% w/v, between about 50.0 to about 55.0% w/v, between about 55.0 to about 60.0% w/v, between about 60.0 to about 65.0% w/v, between about 65.0 to about 70.0% w/v, between about 70.0 to about 75.0% w/v, between about 75.0 to about 80.0% w/v of a solvent, e.g., NMP, a buffer or any other co-solvent.

It is noted that any one, or any combination, of any of the components disclosed herein may be added to the liquid pharmaceutical composition of the invention.

The liquid pharmaceutical compositions of the invention may be in the form of a solution, gel, cream, emulsion, or suspension. According to some embodiments, the liquid pharmaceutical compositions of the invention may be dried to provide a solid, e.g., by lyophilization, wherein the dried material, e.g., the lyophilizate, may be constituted to provide a liquid composition, e.g., by the addition of a solvent, e.g., water. Antioxidants, surfactants and the like may also be added when the dried composition is constituted. According to some embodiments, the dried composition is reconstituted using a dedicated solution comprising, e.g., a solvent, an antioxidant, a surfactant and any other required excipients. According to some embodiments, the liquid pharmaceutical composition of the invention is an aqueous composition.

The liquid pharmaceutical compositions of the invention may be formulated for any suitable route of administration, e.g., for parenteral administration, e.g., by bolus administration or continuous administration. The liquid pharmaceutical composition of the invention may be formulated for subcutaneous, transdermal, intradermal, transmucosal, intravenous, intraarterial, intramuscular, intraperitoneal, intratracheal, intrathecal, intraduodenal, intrapleural, intranasal, sublingual, buccal, intestinal, intraduodenally, rectal, intraocular, or oral administration. The compositions may also be formulated for inhalation, or for direct absorption through mucous membrane tissues.

Further embodiments of the invention are directed to a process for preparing a liquid pharmaceutical composition, wherein said process comprises:
mixing a levodopa amino acid conjugate (LDAA) of the general formula (I):

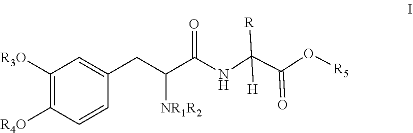

in a pharmaceutically acceptable salt form with at least one solvent thereby forming a solution, gel, cream, emulsion, or suspension; and
adjusting the pH of the solution, gel, cream, emulsion, or suspension, to a physiologically acceptable pH value, thereby providing the liquid pharmaceutical composition, wherein:

R is an amino acid side chain;

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

According to some embodiments, the process comprises mixing an LDAA compound of Formula (I) in a pharmaceutically acceptable salt form with at least one solvent, thereby forming a solution. According to some embodiments, the process comprises mixing an LDAA compound of Formula (I) in a pharmaceutically acceptable solid salt form with at least one solvent. According to some embodiments, the process of the invention includes further mixing the LDAA compound of Formula (I) with any additional active pharmaceutical ingredients and/or pharmaceutically acceptable excipients, as detailed regarding the liquid pharmaceutical composition of the invention.

According to some embodiments, the process comprises mixing a salt form of an LDAA with at least one solvent, wherein each one of R₁, R₂, R₃, R₄ and R₅ are H. According to some embodiments, the process comprises mixing a salt form of an LDAA with at least one solvent, wherein the salt is a TFA salt, an HCl salt fumaric acid salt, lactate salt, maleic acid salt, gluceptic acid salt, phosphoric acid salt, sulfuric acid salt, HBr salt, nitric acid salt, acetic acid salt, propionic acid salt, hexanoic acid salt, cyclopentanepropionic acid salt, glycolic acid salt, pyruvic acid salt, lactic acid salt, hippuric acid salt, methanesulfonic acid salt, ascorbic acid salt, malonic acid salt, oxalic acid salt, maleic acid salt, tartaric acid salt, citric acid salt, succinic acid salt, benzoic acid salt, cinnamic acid salt, a sulfonic acid salt, lauryl sulfuric acid salt, gluconic acid salt, glutamic acid salt, hydroxynaphthoic acid salt, salicylic acid salt, stearic acid salt, muconic acid salt, an alkali metal salt, such as lithium salt, sodium salt or potassium salt, an alkaline earth metal salt, such as calcium salt or magnesium salt, an aluminum salt, an ethanolamine salt, diethanolamine salt, triethanolamine salt, N-methylglucamine salt, dicyclohexylamine salt, adipate salt, alginate salt, ascorbate salt, aspartate salt, benzenesulfonate salt, bisulfate salt, borate salt, butyrate salt, camphorate butyrate salt, camphorsulfonate butyrate salt, digluconate butyrate salt, dodecylsulfate butyrate salt, ethanesulfonate butyrate salt, glucoheptonate butyrate salt, glycerophosphate butyrate salt, gluconate butyrate salt, hemisulfate butyrate salt, heptanoate butyrate salt, hydroiodide butyrate salt, 2-hydroxy-ethanesulfonate butyrate salt, lactobionate butyrate salt, laurate butyrate salt, methanesulfonate butyrate salt, 2-naphthalenesulfonate butyrate salt, nicotinate butyrate salt, oleate butyrate salt, palmitate butyrate salt, pamoate butyrate salt, pectinate butyrate salt, persulfate butyrate salt, 3-phenylpropionate butyrate salt, phosphate butyrate salt, picrate butyrate salt, pivalate butyrate salt, tartrate butyrate salt, thiocyanate butyrate salt, p-toluenesulfonate butyrate salt, undecanoate butyrate salt, valerate salts, or any combination thereof.

Further embodiments of the invention are directed to a liquid pharmaceutical composition prepared according to the process of the invention.

Some embodiments of the invention are directed to a liquid pharmaceutical composition in which the LDAA compound, an enantiomer, diastereomer, racemate, ion, zwitterion, pharmaceutically acceptable salt thereof, or any combination thereof has a solubility of between about 100 to about 1000 mg/L at a physiologically acceptable pH. According to some embodiments, the solubility of the LDAA compound, an enantiomer, diastereomer, racemate, ion, zwitterion, pharmaceutically acceptable salt thereof, or any combination thereof, is between about 100 to about 200 mg/L, between about 200 to about 300 mg/L, between about 300 to about 400 mg/L, between about 400 to about 500 mg/L, between about 500 to about 600 mg/L, between about 600 to about 700 mg/L, between about 700 to about 800 mg/L, between about 800 to about 900 mg/L, between about 900 to about 1000 mg/L, at a physiologically acceptable pH.

Further embodiments of the invention are directed to a method of treating neurodegenerative conditions and/or conditions characterized by reduced levels of dopamine in the brain, wherein the method comprises administering a liquid pharmaceutical composition, wherein the liquid pharmaceutical composition comprises a levodopa amino acid conjugate (LDAA) of the general formula (I):

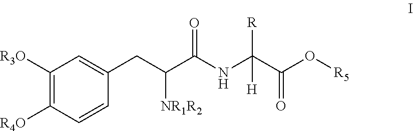

an enantiomer, diastereomer, racemate, ion, zwitterion, pharmaceutically acceptable salt thereof, or any combination thereof, wherein

R is an amino acid side chain;

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

According to some embodiments, neurodegenerative conditions and/or conditions characterized by reduced levels of dopamine in the brain are selected from Parkinson's disease, secondary parkinsonism, Huntington's disease, Parkinson's like syndrome, progressive supranuclear palsy (PSP), multiple system atrophy (MSA), amyotrophic lateral sclerosis (ALS), Shy-Drager syndrome, dystonia, Alzheimer's disease, Lewy body dementia (LBD), akinesia, bradykinesia, and hypokinesia, conditions resulting from brain injury, including carbon monoxide or manganese intoxication, conditions associated with a neurological disease or disorder, including alcoholism, opiate addiction, and erectile dysfunction. According to some embodiments, the neurodegenerative condition and/or condition characterized by reduced levels of dopamine in the brain is Parkinson's disease.

According to some embodiments, the method of the invention comprises administering the LDAA compound of Formula (I), an enantiomer, diastereomer, racemate, ion, zwitterion, pharmaceutically acceptable salt thereof, or any combination thereof, or any combination of two or more LDAA compounds, enantiomers, diastereomers, racemates, ions, zwitterions, pharmaceutically acceptable salts thereof, or any combination thereof, concomitantly with an additional active ingredient, such as a decarboxylase inhibitor, e.g., carbidopa, a COMT inhibitor, a MAO inhibitor, or any combination thereof. According to some embodiments, the LDAA compound is administered together with a decarboxylase inhibitor, e.g., carbidopa, wherein the LDAA compound and the decarboxylase inhibitor are administered in a single formulation.

According to some embodiments, the method of the invention comprises administering the liquid pharmaceutical composition substantially continuously. According to some embodiments, the liquid pharmaceutical composition is administered subcutaneously. According to some embodiments, the liquid pharmaceutical composition is administered subcutaneously via a designated pump device.

Embodiments of a designated pump may be, for example, any of the pump embodiments disclosed in U.S. 62/529,784, U.S. 62/576,362, PCT/IB2018/054962, U.S. Ser. No. 16/027,804, U.S. Ser. No. 16/027,710, U.S. Ser. No. 16/351,072, U.S. Ser. No. 16/351,076, U.S. Ser. No. 16/351,061, USD 29/655583, USD 29/655587, USD 29/655589, USD 29/655591, USD 29/655592, USD 29/655594, USD 29/655597, and U.S. 62/851,903, all of which are incorporated herein by reference in their entirety.

According to some embodiments, the method of the invention comprises administering the liquid pharmaceutical composition at one site, two sites, or three or more sites, wherein the position of the sites may be changed at any appropriate, possibly pre-determined, intervals. Once administered via a specific site, according to some embodiments, the administration via the same site, or the vicinity of that site, may be only after a, possibly predefined, period of time. According to some embodiments, the position of any one of the sites is changed after 12, 24, 36, 48, 60 or 72 hours. According to some embodiments, the position of the site is changed after 4, 5, 6 or 7 days. According to some embodiments, the position of the site is changed after two three or four weeks. According to some embodiments, the position of the site is changed when required or desired, e.g., according to subjective data received from the patient and/or according to objective data received, e.g., from sensors located at, or in the vicinity of, the injection site(s).

According to some embodiments, the administrated volume and/or the administration rate is identical in all or at least two of the sites. According to other embodiments, the administration rate and/or administrated volume differ from site to site. Each site may be controlled independently or otherwise, all sites may be controlled dependently on one another.

According to some embodiments, the method of the invention comprises subcutaneously administrating between about 1 to about 15 ml of the liquid pharmaceutical composition of the invention over the course of 24 hours. According to some embodiments, the method of invention comprises subcutaneously administrating between about 1 to about 2, between about 2 to about 3, between about 3 to about 4, between about 4 to about 5, between about 5 to about 6, between about 6 to about 7, between about 7 to about 8, between about 8 to about 9, between about 9 to about 10, between about 10 to about 11, between about 11 to about 12, between about 12 to about 13, between about 13 to about 14, between about 14 to about 15 ml over the course of 24 hours.

It is noted that the administration rate may be constant over the course of 24 hours or may change over the course of 24 hours. For instance, according to some embodiments, there may be a certain rate for high activity/day hours and a different rate for low activity/night hours. The high activity/day hours may be, e.g., about 15, about 16, about 17, about 18 or about 19 hours, while the low activity night hours may be about 9, about 8, about 7, about 6 or about 5 hours, respectively. According to some embodiments, the high activity/day rate is implemented for about 18 hours, while the low activity/night rate is implemented for about 6 hours. According to some embodiments, the high activity/day rate is implemented for about 16 hours, while the low activity/night rate is implemented for about 8 hours.

The administration rate may be between about 0.01 mL/site/hour to about 1 mL/site/hour. According to some embodiments, the administration rate is between about 0.01-0.02 mL/site/hour. According to some embodiments, the administration rate is between about 0.02-0.03 mL/site/hour. According to some embodiments, the administration rate is between about 0.03-0.04 mL/site/hour. According to some embodiments, the administration rate is between about 0.04-0.05 mL/site/hour. According to some embodiments, the administration rate is between about 0.05-0.06 mL/site/hour. According to some embodiments, the administration rate is between about 0.06-0.07 mL/site/hour. According to some embodiments, the administration rate is between about 0.07-0.08 mL/site/hour. According to some embodiments, the administration rate is between about 0.08-0.09 mL/site/hour. According to some embodiments, the administration rate is between about 0.09-0.1 mL/site/hour. According to some embodiments, the administration rate is between about 0.1-0.15 mL/site/hour. According to some embodiments, the administration rate is between about 0.15-0.2 mL/site/hour. According to some embodiments, the administration rate is between about 0.2-0.25 mL/site/hour. According to some embodiments, the administration rate is between about 0.25-0.3 mL/site/hour. According to some embodiments, the administration rate is between about 0.3-0.35 mL/site/hour. According to some embodiments, the administration rate is between about 0.35-0.4 mL/site/hour. According to some embodiments, the administration rate is between about 0.4-0.45 mL/site/hour. According to some embodiments, the administration rate is between about 0.45-0.5 mL/site/hour. According to some embodiments, the administration rate is between about 0.5-0.55 mL/site/hour. According to some embodiments, the administration rate is between about 0.55-0.6 mL/site/hour. According to some embodiments, the administration rate is between about 0.6-0.65 mL/site/hour. According to some embodiments, the administration rate is between about 0.65-0.7 mL/site/hour. According to some embodiments, the administration rate is between about 0.7-0.75 mL/site/hour. According to some embodiments, the administration rate is between about 0.75-0.8 mL/site/hour. According to some embodiments, the administration rate is between about 0.8-0.85 mL/site/hour. According to some embodiments, the administration rate is between about 0.85-0.9 mL/site/hour. According to some embodiments, the administration rate is between about 0.9-0.95 mL/site/hour. According to some embodiments, the administration rate is between about 0.95-1.0 mL/site/hour.

According to some embodiments, the administration rate in the low activity/night hours is between about 0.01-0.15 mL/site/hour. According to some embodiments, the administration rate in the low activity/night hours is between about 0.01-0.02 mL/site/hour. According to some embodiments, the administration rate in the low activity/night hours is between about 0.02-0.03 mL/site/hour. According to some embodiments, the administration rate in the low activity/night hours is between about 0.03-0.04 mL/site/hour. According to some embodiments, the administration rate in the low activity/night hours is between about 0.04-0.05 mL/site/hour. According to some embodiments, the administration rate in the low activity/night hours is between about 0.05-0.06 mL/site/hour. According to some embodiments, the administration rate in the low activity/night hours is between about 0.06-0.07 mL/site/hour. According to some embodiments, the administration rate in the low activity/night hours is between about 0.07-0.08 mL/site/hour. According to some embodiments, the administration rate in the low activity/night hours is between about 0.08-0.09 mL/site/hour. According to some embodiments, the administration rate in the low activity/night hours is between about 0.09-0.1 mL/site/hour. According to some embodiments, the administration rate in the low activity/night hours is between about 0.1-0.11 mL/site/hour. According to some embodiments, the administration rate in the low activity/night hours is between about 0.11-0.12 mL/site/hour. According to some embodiments, the administration rate in the low activity/night hours is between about 0.12-0.13 mL/site/hour. According to some embodiments, the administration rate in the low activity/night hours is between about 0.13-0.14 mL/site/hour. According to some embodiments, the administration rate in the low activity/night hours is between about 0.14-0.15 mL/site/hour. According to some embodiments, the administration rate in the low activity/night hours is about 0.04 mL/site/hour. According to some embodiments, the administration rate in the high activity/day hours is between about 0.15-1.0 mL/site/hour. According to some embodiments, the administration rate in the high activity/day hours is between about 0.15-0.2 mL/site/hour. According to some embodiments, the administration rate in the high activity/day hours is between about 0.2-0.25 mL/site/hour. According to some embodiments, the administration rate in the high activity/day hours is between about 0.25-0.3 mL/site/hour. According to some embodiments, the administration rate in the high activity/day hours is between about 0.3-0.35 mL/site/hour. According to some embodiments, the administration rate in the high activity/day hours is between about 0.35-0.4 mL/site/hour. According to some embodiments, the administration rate in the high activity/day hours is between about 0.4-0.45 mL/site/hour. According to some embodiments, the administration rate in the high activity/day hours is between about 0.45-0.5 mL/site/hour. According to some embodiments, the administration rate in the high activity/day hours is between about 0.5-0.55 mL/site/hour. According to some embodiments, the administration rate in the high activity/day hours is between about 0.55-0.6 mL/site/hour. According to some embodiments, the administration rate in the high activity/day hours is between about 0.6-0.65 mL/site/hour. According to some embodiments, the administration rate in the high activity/day hours is between about 0.65-0.7 mL/site/hour. According to some embodiments, the administration rate in the high activity/day hours is between about 0.7-0.75 mL/site/hour. According to some embodiments, the administration rate in the high activity/day hours is between about 0.75-0.8 mL/site/hour. According to some embodiments, the administration rate in the high activity/day hours is between about 0.8-0.85 mL/site/hour. According to some embodiments, the administration rate in the high activity/day hours is between about 0.85-0.9 mL/site/hour. According to some embodiments, the administration rate in the high activity/day hours is between about 0.9-0.95 mL/site/hour. According to some embodiments, the administration rate in the high activity/day hours is between about 0.95-1.0 mL/site/hour. According to some embodiments, the administration rate in the high activity/day hours is about 0.32 mL/site/hour.

It is further noted that the administrated volume and/or administration rate may be constant throughout the treatment, or may vary during different hours of the day, between different days, weeks or months of treatment, and the like. According to some embodiments, the patient is monitored, e.g., independently, by a caretaker, or electronically, e.g., by sensors, possibly found in a dedicated device, e.g., a watch-like device, the administration pump, and the like. According to such embodiments, the administration volume and/or rate are determined according to data received from such monitoring.

Some embodiments are directed to a method for administering a bolus subcutaneous injection of the liquid pharmaceutical composition of the invention. According to some embodiments, the bolus injection comprises between about 0.5 to about 2.0 mL/Kg of the liquid pharmaceutical composition. According to some embodiments, the bolus injection comprises between about 0.5 to about 0.75 mL/Kg of the liquid pharmaceutical composition. According to some embodiments, the bolus injection comprises between about 0.75 to about 1.0 mL/Kg of the liquid pharmaceutical composition. According to some embodiments, the bolus injection comprises between about 1.0 to about 1.25 mL/Kg of the liquid pharmaceutical composition. According to some embodiments, the bolus injection comprises between about 1.25 to about 1.5 mL/Kg of the liquid pharmaceutical composition. According to some embodiments, the bolus injection comprises between about 1.5 to about 1.75 mL/Kg of the liquid pharmaceutical composition. According to some embodiments, the bolus injection comprises between about 1.75 to about 2.0 mL/Kg of the liquid pharmaceutical composition. According to some embodiments, the bolus injection comprises between about 0.75 to about 1.25 mL/Kg of the liquid pharmaceutical composition. According to some embodiments, the bolus injection comprises about 1.0 mL/Kg of the liquid pharmaceutical composition.

The bolus subcutaneous injection may be administered at any time point in relation to any possible continuous subcutaneous administrations, e.g., prior to, during, or after the continuous administration.

According to some embodiments, the administered dose may be doubled, tripled or more, by using more than one pump, more than one injection site for each pump, and the like.

According to some embodiments, the liquid pharmaceutical compositions are administered for a defined period of time, e.g., days, weeks, months, or years. According to some embodiments, the liquid pharmaceutical compositions are administered endlessly, for the treatment of a chronic condition.

Further embodiments of the invention are directed to a liquid pharmaceutical composition for use in the treatment of neurodegenerative conditions and/or conditions characterized by reduced levels of dopamine in the brain, wherein the liquid pharmaceutical composition comprises a levodopa amino acid conjugate (LDAA) of the general formula (I):

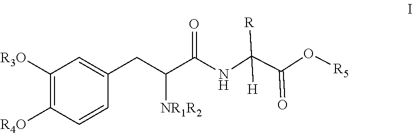

an enantiomer, diastereomer, racemate, ion, zwitterion, pharmaceutically acceptable salt thereof, or any combination thereof, wherein

R is an amino acid side chain;

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

According to some embodiments, the liquid pharmaceutical composition is for use in the treatment of Parkinson's disease, secondary parkinsonism, Huntington's disease, Parkinson's like syndrome, progressive supranuclear palsy (PSP), multiple system atrophy (MSA), amyotrophic lateral sclerosis (ALS), Shy-Drager syndrome, dystonia, Alzheimer's disease, Lewy body dementia (LBD), akinesia, bradykinesia, and hypokinesia, conditions resulting from brain injury, including carbon monoxide or manganese intoxication, conditions associated with a neurological disease or disorder, including alcoholism, opiate addiction, and erectile dysfunction. Certain embodiments of the invention are directed to the liquid pharmaceutical composition of the invention in the treatment of Parkinson's disease.

The composition for use according to the invention may include any of the additional materials, the amounts of any of the materials, as detailed herein regarding the embodiments of the composition of the invention. Further, the form, pH, and the like, of the compositions for use according to the invention may be as detailed herein regarding the embodiments of the composition of the invention. In addition, the composition of the invention may be used together with a COMT inhibitor, MAO inhibitor, or any other active ingredient, as detailed herein.

Further embodiments of the invention are directed to a levodopa amino acid conjugate (LDAA) of the general formula (III):

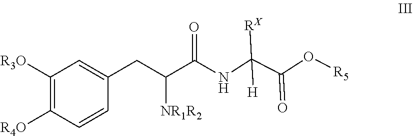

an enantiomer, diastereomer, racemate, ion, zwitterion, pharmaceutically acceptable salt thereof, or any combination thereof, wherein

R^{X} is an amino acid side chain or an O-phosphorylated amino acid side chain thereof;

R1 and R2 each independently is selected from the group consisting of H, (C1-C6)alkyl, (C2-C6)alkenyl, (C2-C6)alkynyl, C3-C6cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R3 and R4 each independently is selected from the group consisting of H, (C1-C3)alkyl, C3-C6cycloalkyl, phenyl, or —P(═O)(OR′)₂;

R5 is selected from the group consisting of H, (C1-C3)alkyl, C3-C6cycloalkyl and phenyl;

R′ is each independently selected from the group consisting of H, (C1-C6)alkyl, (C2-C6)alkenyl, C3-C6cycloalkyl, phenyl, and heteroaryl bonded to the nitrogen through a ring carbon; and

R″ is selected from the group consisting of a (C1-C6)alkyl, (C2-C6)alkenyl, and (C2-C6)alkynyl.

Further embodiments of the invention are directed to a levodopa amino acid conjugate (LDAA) of the general formula (III):

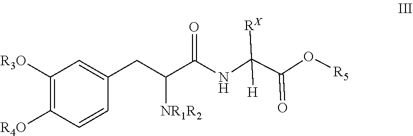

an enantiomer, diastereomer, racemate, ion, zwitterion, pharmaceutically acceptable salt thereof, or any combination thereof, wherein

R^{X} is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, ornithine, lanthionine and 3,4-dihydroxyphenylalanine side chain; or a O-phosphorylated amino acid side chain thereof;

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

For example, in embodiments described herein, the amino acid side chain in R^{X} can be:

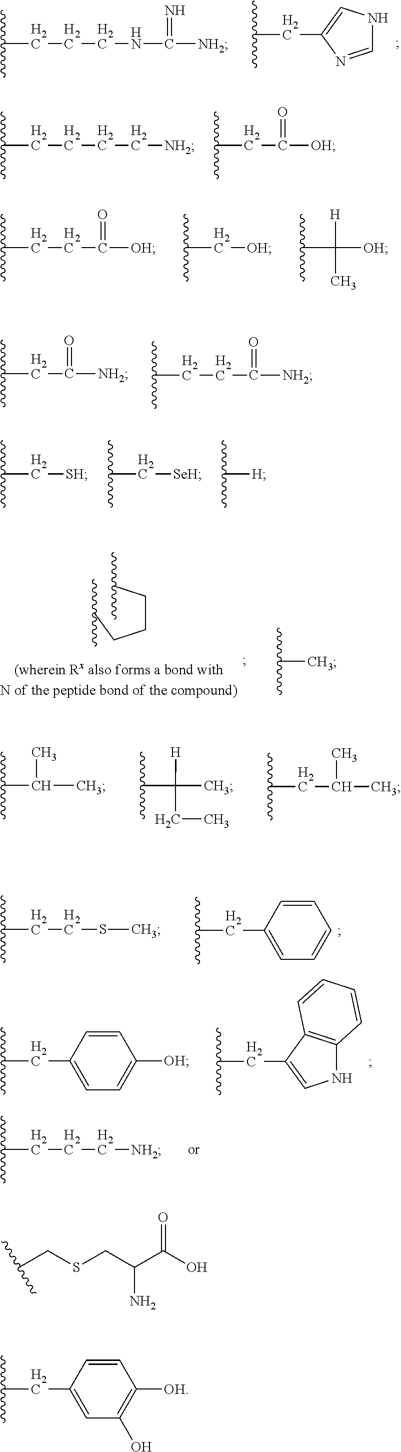

According to some embodiments of the general formula (III), R^{X} is an amino acid side chain selected from the group consisting of arginine, lysine, serine, glycine, alanine, valine, phenylalanine, tyrosine, ornithine and 3,4-dihydroxyphenylalanine; or a n-phosphorylated amino acid side chain thereof.

According to some embodiments of the general formula (III), each one of R₁, R₂ and R₅ are H; R₃, and R₄ independently are H or —P(═O)(OR′)₂; and R′ is H.

According to preferable embodiments of the general formula (III), R^{X} is an amino acid side chain selected from the group consisting of arginine, lysine, serine, glycine, alanine, valine, phenylalanine, tyrosine, ornithine and 3,4-dihydroxyphenylalanine; or an O-phosphorylated amino acid side chain thereof; each one of R₁, R₂ and R₅ are H; R₃, and R₄ independently are H or —P(═O)(OR′)₂; and R′ is H.

Preferable embodiments are listed below as example:

TABLE 1

| Example | Compound name |
|---|---|
| 4 | (2S)-6-amino-2-[[(2S)-2-amino-3-(3-hydroxy-4- |
| | phosphonooxyphenyl)propanoyl] amino]hexanoic acid |
| 5 | (2S)-2-[[(2S)-2-amino-3-(3-hydroxy-4- |
| | phosphonooxyphenyl)propanoyl]amino]-3-(4- |
| | hydroxyphenyl)propanoic acid |
| 6 | (2S)-2-[[(2S)-2-amino-3-(3-hydroxy-4- |
| | phosphonooxyphenyl)propanoyl]amino]-5-carbamimidamide |
| | pentanoic acid; hydrochloride |
| 7 | (2S)-2-amino-3-(3-hydroxy-4- |
| | phosphonooxyphenyl)propionamide |
| 8 | (2S)-2-[[(2S)-2-amino-3-(3-hydroxy-4- |
| | phosphonooxyphenyl)propanoyl] amino]propanoic acid |
| 9 | 2-[[(2S)-2-amino-3-(3-hydroxy-4- |
| | phosphonooxyphenyl)propanoyl]amino]acetic acid |
| 10 | 2-[[(2S)-2-amino-3-(3-hydroxy-4- |
| | phosphonooxyphenyl)propanoyl]amino]ethanesulfonic acid |
| 11 | (2S)-2-[[(2S)-2-amino-3-(3-hydroxy-4- |
| | phosphonooxyphenyl)propanoyl]amino]-3-phenylpropanoic |
| | acid |
| 12 | (2S)-2-[[(2S)-2-amino-3-(3,4- |
| | dihydroxyphenyl)propanoyl]amino]-3- |
| | phosphonooxypropanoic acid |
| 13 | (2S)-2-[[(2S)-2-amino-3-(3-hydroxy-4- |
| | phosphonooxyphenyl)propanoyl]amino]-3-(3,4- |
| | dihydroxyphenyl)propanoic acid |
| 14 | (2S)-2-amino-6-[[(2S)-2-amino-3-(3-hydroxy-4- |
| | phosphonooxyphenyl)propanoyl]amino]hexanoic acid |
| 15 | (2S)-5-amino-2-[[(2S)-2-amino-3-(3-hydroxy-4- |
| | phosphonooxyphenyl)propanoyl]amino]pentanoic acid |
| 16 | (2S)-2-amino-5-[[(2S)-2-amino-3-(3-hydroxy-4- |
| | phosphonooxyphenyl)propanoyl] amino]pentanoic acid |
| 17 | (2S)-2-[[(2S)-2-amino-3-(3-hydroxy-4- |
| | phosphonooxyphenyl)propanoyl]amino]-3-hydroxypropanoic |
| | acid |
| 18 | (2S)-2-[[(2S)-2-amino-3-(3-hydroxy-4- |
| | phosphonooxyphenyl)propanoyl]amino]-3-methylbutanoic |
| | acid |
| 19 | (2S)-2-[[(2S)-2-amino-3-(3-hydroxy-4- |
| | phosphonooxyphenyl)propanoyl]amino]-3-(3-hydroxy-4- |
| | phosphonooxyphenyl)propanoic acid |
| 20 | (2S)-2-[[(2S)-2-amino-3-(4-hydroxy-3- |
| | phosphonooxyphenyl)propanoyl]amino]-3-(4-hydroxy-3- |
| | phosphonooxyphenyl))propanoic acid |
| 21 | (2S)-2-[[(2S)-2-amino-3-(3,4- |
| | dihydroxyphenyl)propanoyl]amino]-3-(4- |
| | phosphonooxyphenyl)propanoic acid |

Further embodiments of the invention are directed to a levodopa amino acid conjugate (LDAA) selected from the group consisting of:
(2S)-2-amino-3-(3-hydroxy-4-phosphonooxyphenyl)propionamide;
2-[[(2S)-2-amino-3-(3-hydroxy-4-phosphonooxyphenyl)propanoyl]amino]ethanesulfonic acid;
(2S)-2-amino-6-[[(2S)-2-amino-3-(3-hydroxy-4-phosphonooxyphenyl)propanoyl]amino]hexanoic acid; or
(2S)-2-amino-5-[[(2S)-2-amino-3-(3-hydroxy-4-phosphonooxyphenyl)propanoyl]amino]pentanoic acid.

Embodiments of the invention are directed to a levodopa-lanthionine conjugate (LD-LA) of the general formula (II-1) or (II-2):

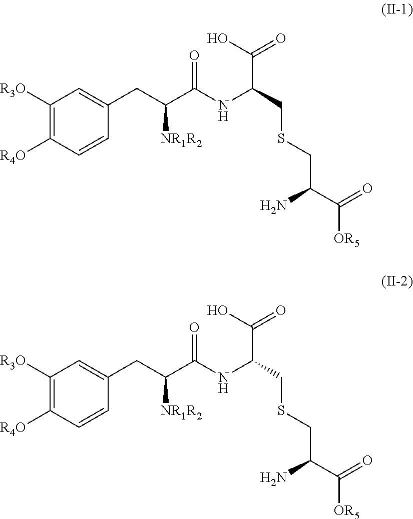

an enantiomer, diastereomer, racemate, ion, zwitterion, pharmaceutically acceptable salt thereof, or any combination thereof, wherein:

R₁ and R₂ each independently is selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, or —O—C(═O)—R″;

R₃ and R₄ each independently is selected from the group consisting of H, (C₁-C₃)alkyl, C₃-C₆cycloalkyl, phenyl, or —P(═O)(OR′)₂;

R₅ is selected from the group consisting of H, (C₁-C₃)alkyl, C₃-C₆cycloalkyl and phenyl;

R′ is each independently selected from the group consisting of H, (C₁-C₆)alkyl, (C₂-C₆)alkenyl, C₃-C₆cycloalkyl, phenyl, and heteroaryl bonded to the nitrogen through a ring carbon; and

R″ is selected from the group consisting of a (C₁-C₆)alkyl, (C₂-C₆)alkenyl, and (C₂-C₆)alkynyl.

According to some embodiments, each one of R₁, R₂, R₃, R₄ and R₅ are H.

The compound represented by the general formula [III] of the present invention may be produced, for example, as follows:

### Synthesis Method (A)

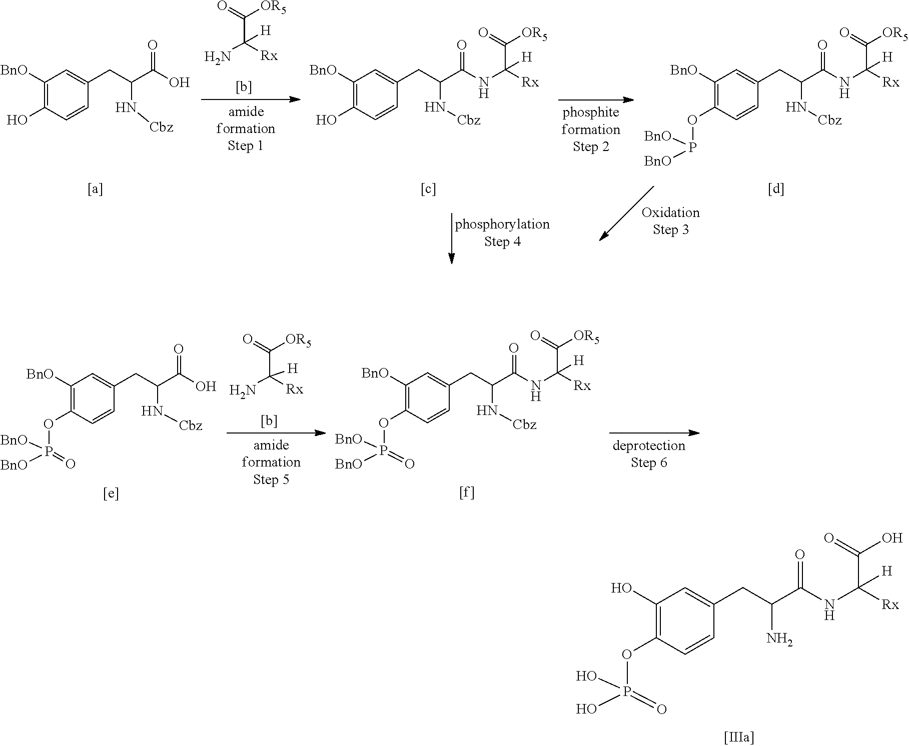

wherein the symbols have the same meaning as above.

Among the target compounds [III] of the present invention, the compound represented by the general formula [Ma] can be produced, for example, as follows. The compound [a] and the compound [b] are subjected to a condensation reaction to obtain the compound [c], and then, the compound [c] is subjected to phosphite esterification and oxidation or is subjected to phosphate esterification, and thereby, the compound [f] is obtained. On the other hand, the compound [f] can also be obtained by condensing the compound [e] and the compound [b]. The compound [IIIa] can be produced by deprotecting the compound [f] thus obtained.

### Step 1:

The condensation of the compound [a] with the compound [b] or a salt thereof can be carried out according to a common method in a suitable solvent in the presence or absence of a base, in the presence or absence of a condensing agent, and in the presence or absence of an activating agent. As the solvent, any solvent that does not affect the present reaction may be used. Examples of the solvent include: ethers such as tetrahydrofuran and 1,4-dioxane; amides such as N,N-dimethylformamide and N-methylpyrrolidone; nitriles such as acetonitrile; halogenated aliphatic hydrocarbons such as chloroform and dichloromethane; aromatic hydrocarbons such as toluene; or a mixture of these compounds. Examples of the base include triethylamine, diisopropylethylamine, diazabicycloundecene and the like. Examples of the condensing agent include O-(7-azabenzotriazol-1-yl)-N,N,N′,N′-tetramethyluronium hexafluorophosphate (HATU), 1-ethyl-3-(3-dimethylaminopropyl)carbodiimide hydrochloride, 1-(3-dimethylaminopropyl)-3-ethylcarbodiimide hydrochloride, and the like. Examples of the activating agent include 1-hydroxy-7-azabenzotriazole (HOAt), 1-hydroxybenzotriazole (HOBt), 4-dimethylaminopyridine and the like.

An amount of the compound [b] to be used can be 1.0-5.0 equivalents, preferably 1.0-2.0 equivalents, in molar ratio with respect to the compound [a].

An amount of the base to be used can be 1.0-5.0 equivalents, preferably 1.0-2.0 equivalents, in molar ratio with respect to the compound [a].

An amount of the condensing agent to be used can be 1.0-5.0 equivalents, preferably 1.0-2.5 equivalents, in molar ratio with respect to the compound [a].

An amount of the activating agent to be used can be 1.0-5.0 equivalents, preferably 1.0-2.5 equivalents, in molar ratio with respect to the compound [a].

The present reaction can be carried out at room temperature—under heating, for example, at room temperature −80° C., preferably at room temperature −50° C.

### Step 2

The condensation of the compound [c] and a phosphite esterifying agent can be carried out according to a common method in a suitable solvent in the presence of an activating agent. As the solvent, any solvent that does not affect the present reaction may be used. Examples of the solvent include: nitriles such as acetonitrile; halogenated aliphatic hydrocarbons such as chloroform and dichloromethane; or a mixture of these compounds. An example of the phosphite esterifying agent is dibenzyl N,N-diisopropyl phosphoramidite. An example of the activating agent is 1-tetrazole.

An amount of the phosphite esterifying agent to be used can be 1.0-5.0 equivalents, preferably 1.5-3.0 equivalents, in molar ratio with respect to the compound [c].

An amount of the activating agent to be used can be 1.0-5.0 equivalents, preferably 1.5-3.0 equivalents, in molar ratio with respect to the compound [c].

The present reaction can be carried out under ice-cooling—under heating, for example, at 0° C.-80° C., preferably at room temperature −50° C.

### Step 3

The oxidation of the compound [d] can be carried out according to a common method in a suitable solvent in the presence of an oxidizing agent. As the solvent, any solvent that does not affect the present reaction may be used. Examples of the solvent include: nitriles such as acetonitrile; halogenated aliphatic hydrocarbons such as chloroform and dichloromethane; or a mixture of these compounds. Examples of the oxidizing agent include a hydrogen peroxide solution, tert-butyl hydroperoxide, metachloroperbenzoic acid, and the like.

An amount of the oxidizing agent to be used can be 1.0-5.0 equivalents, preferably 1.5-3.0 equivalents, in molar ratio with respect to the compound [d].

The present reaction can be carried out under ice cooling—at room temperature, preferably under ice cooling.

### Step 4

The condensation of the compound [c] and a phosphate esterifying agent can be carried out according to a common method in a suitable solvent in the presence or absence of a base. As the solvent, any solvent that does not affect the present reaction may be used. Examples of the solvent include: halogenated aliphatic hydrocarbons such as chloroform and dichloromethane; or a mixture of these compounds. Examples of the phosphate esterifying agent include dibenzylphosphoryl chloride, tetrabenzyl pyrophosphate, and the like. Examples of the base include: alkali metal alkoxides such as sodium t-butoxide and potassium t-butoxide; alkylamines such as triethylamine and diisopropylethylamine; and the like.

An amount of the phosphate esterifying agent to be used can be 1.0-5.0 equivalents, preferably 1.5-3.0 equivalents, in molar ratio with respect to the compound [c].

An amount of the base to be used can be 1.0-5.0 equivalents, preferably 1.5-3.0 equivalents, in molar ratio with respect to the compound [c].

The present reaction can be carried out at room temperature—under heating, for example, at room temperature −100° C., preferably at room temperature −70° C.

### Step 5

The condensation of the compound [e] with the compound [b] or a salt thereof can be carried out according to a common method in a suitable solvent in the presence or absence of a base, in the presence or absence of a condensing agent, and in the presence or absence of an activating agent. As the solvent, any solvent that does not affect the present reaction may be used. Examples of the solvent include: ethers such as tetrahydrofuran and 1,4-dioxane; amides such as N,N-dimethylformamide and N-methylpyrrolidone; nitriles such as acetonitrile; halogenated aliphatic hydrocarbons such as chloroform and dichloromethane; aromatic hydrocarbons such as toluene; or a mixture of these compounds. Examples of the base include triethylamine, diisopropylethylamine, diazabicycloundecene and the like. Examples of the condensing agent include O-(7-azabenzotriazol-1-yl)-N,N,N′,N′-tetramethyluronium hexafluorophosphate (HATU), 1-ethyl-3-(3-dimethylaminopropyl)carbodiimide hydrochloride, 1-(3-dimethylaminopropyl)-3-ethylcarbodiimide hydrochloride, and the like. Examples of the activating agent include 1-hydroxy-7-azabenzotriazole (HOAt), 1-hydroxybenzotriazole (HOBt), 4-dimethylaminopyridine and the like.

An amount of the compound [b] to be used can be 1.0-5.0 equivalents, preferably 1.0-2.0 equivalents, in molar ratio with respect to the compound [e].

An amount of the base to be used can be 1.0-5.0 equivalents, preferably 1.0-2.0 equivalents, in molar ratio with respect to the compound [e].

An amount of the condensing agent to be used can be 1.0-5.0 equivalents, preferably 1.0-2.5 equivalents, in molar ratio with respect to the compound [e].

An amount of the activating agent to be used can be 1.0-5.0 equivalents, preferably 1.0-2.5 equivalents, in molar ratio with respect to the compound [e].

The present reaction can be carried out at room temperature—under heating, for example, at room temperature −80° C., preferably at room temperature −50° C.

### Step 6

The deprotection of the compound [f] can be carried out according to a common method by a treatment with a catalyst in a suitable solvent in a hydrogen atmosphere.

As the solvent, any solvent that does not affect the present reaction may be used. Examples of the solvent include: ethers such as tetrahydrofuran and 1,4-dioxane; alcohols such as methanol, ethanol and isopropanol; water; or a mixture of these compounds.

Examples of the catalyst include palladium carbon and the like.

The present reaction can be carried out at room temperature—under heating, for example, at room temperature −80° C., preferably at room temperature −50° C.

### Synthesis Method (B)

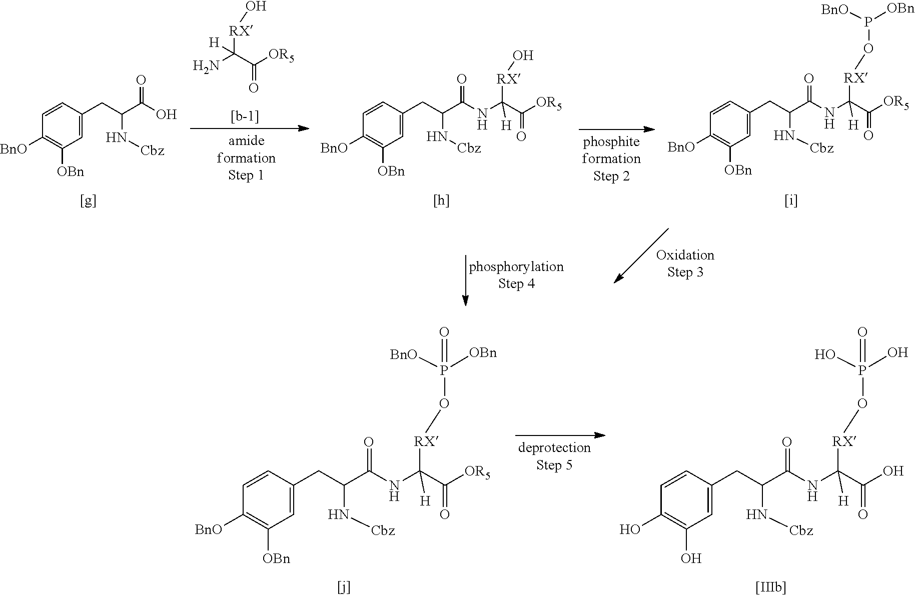

wherein RX′ is amino acid side chain such as serine or tyrosine and the symbols have the same meaning as above.

Among the target compounds [III] of the present invention, the compound represented by the general formula [IIIb] can be produced, for example, as follows. The compound [g] and the compound [b-1] are subjected to a condensation reaction to obtain the compound [h]. The compound [h] is subjected to phosphite esterification to obtain the compound [i], which is then subjected to oxidation to obtain the compound [j], or, the compound [h] is subjected to phosphate esterification to obtain the compound [j]. After that, the compound [IIIb] can be produced by deprotecting the compound [j].

### Step 1

The condensation of the compound [g] or a salt thereof with the compound [b-1] or a salt thereof can be carried out in a similar manner as the reaction of the compound [a] and the compound [b] in the synthesis method (A).

### Step 2

The condensation of the compound [h] and a phosphite esterifying agent can be carried out in a similar manner as the reaction of the compound [c] and a phosphite esterifying agent in the synthesis method (A).

### Step 3

The oxidation of the compound [i] can be carried out in a similar manner as the reaction of the compound [d] in the synthesis method (A).

### Step 4

The condensation of the compound [h] and a phosphate esterifying agent can be carried out in a similar manner as the reaction of the compound [c] and a phosphate esterifying agent in the synthesis method (A).

### Step 5

The deprotection of the compound [j] can be carried out in a similar manner as the reaction of the compound [f] in the synthesis method (A).

Unless explicitly stated, the method embodiments described herein are not constrained to a particular order or sequence. Additionally, some of the described method embodiments or elements thereof can occur or be performed simultaneously, at the same point in time, or concurrently.

It is appreciated that certain features of the invention may also be provided in combination in a single embodiment. Conversely, various elements of the invention that are, for brevity, described in the context of a single embodiment, may also be provided separately or in any suitable sub-combination or as suitable in any other described embodiment of the invention. Further, certain features described in the context of various embodiments are not to be considered essential features of those embodiments, unless the embodiment is inoperative without those elements.

Various embodiments and aspects of the present invention as delineated hereinabove and as claimed in the claims section below may be supported by the following examples; however, they are not to be limited by the examples.

### EXAMPLES

### Example 1—Preparation of Levodopa Amino Acids (LDAA)

Ten LDAA conjugates were prepared for initial screening as trifluoroacetic acid (TFA) salts.

### Preparation of Levodopa Arginine TFA Salt (LD-Arg TFA Salt)

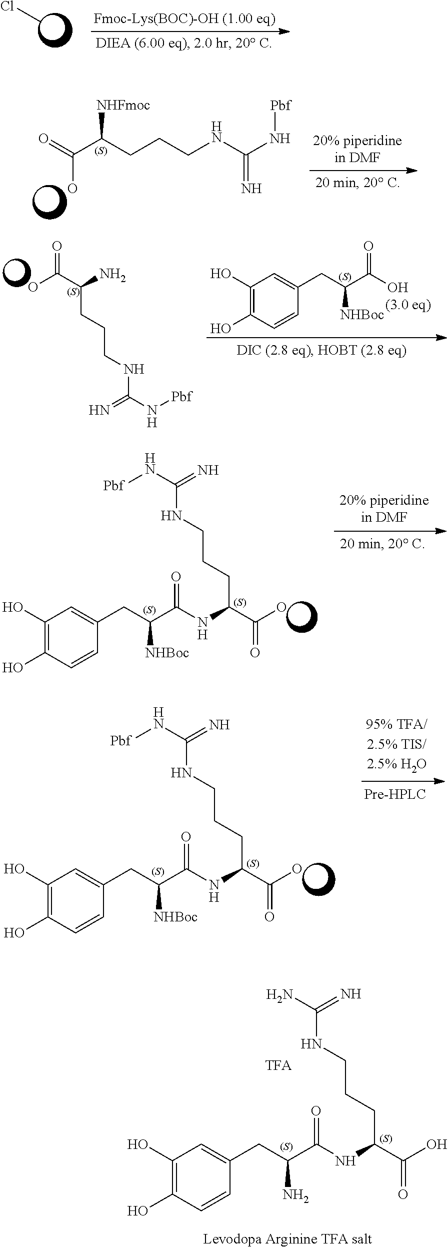

### Preparation of Levodopa Glycine TFA Salt (LD-Gly TFA Salt)

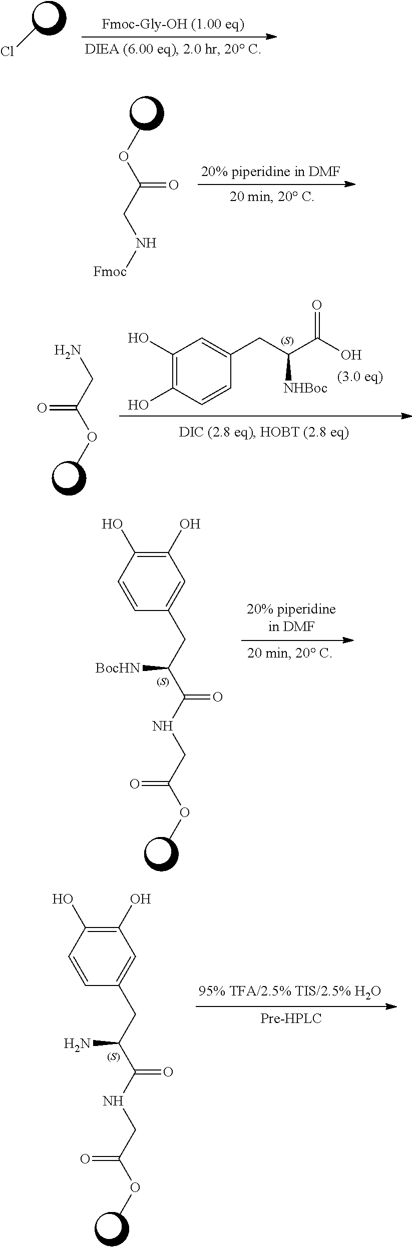

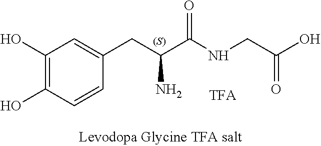

### Preparation of Levodopa Lysine TFA Salt (LD-Lys TFA Salt)

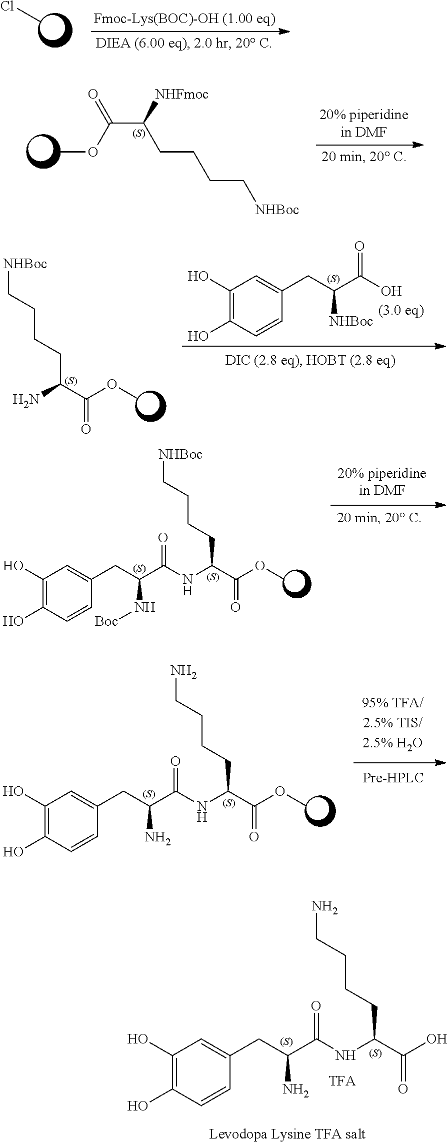

### Preparation of Levodopa Aspartic Acid (LD-Asp)

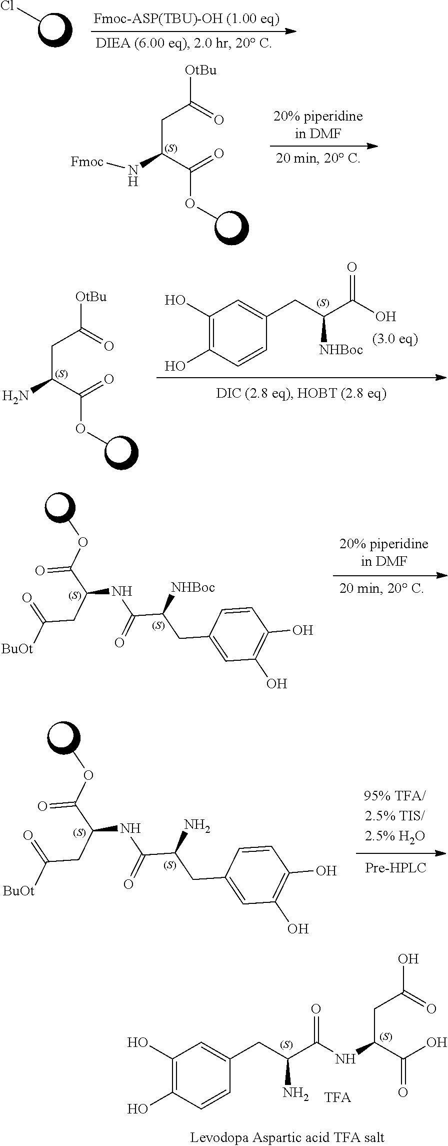

### Preparation of Levodopa Glutamic Acid TFA Salt (LD-Glu TFA Salt)

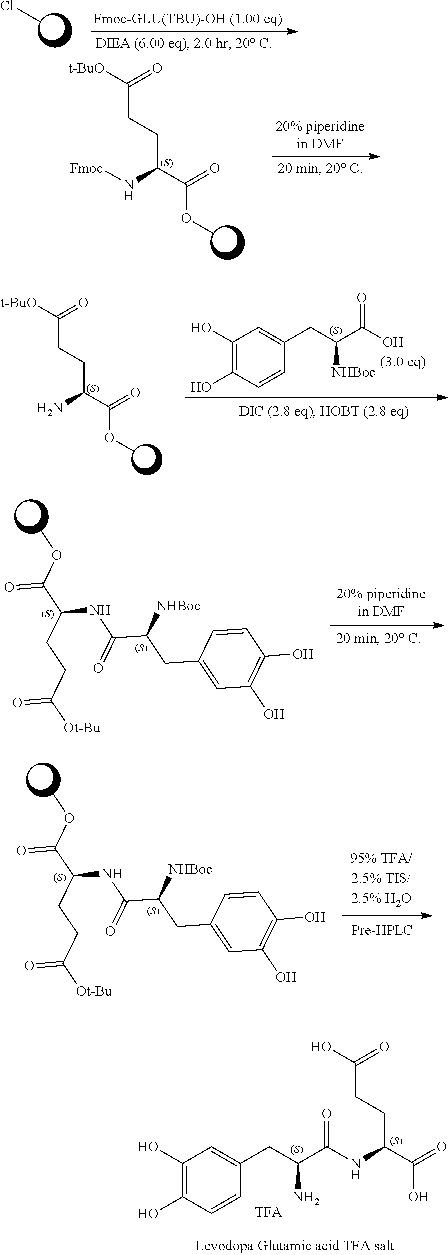

### Preparation of Levodopa Glutamine TFA Salt (LD-Gln TFA Salt)

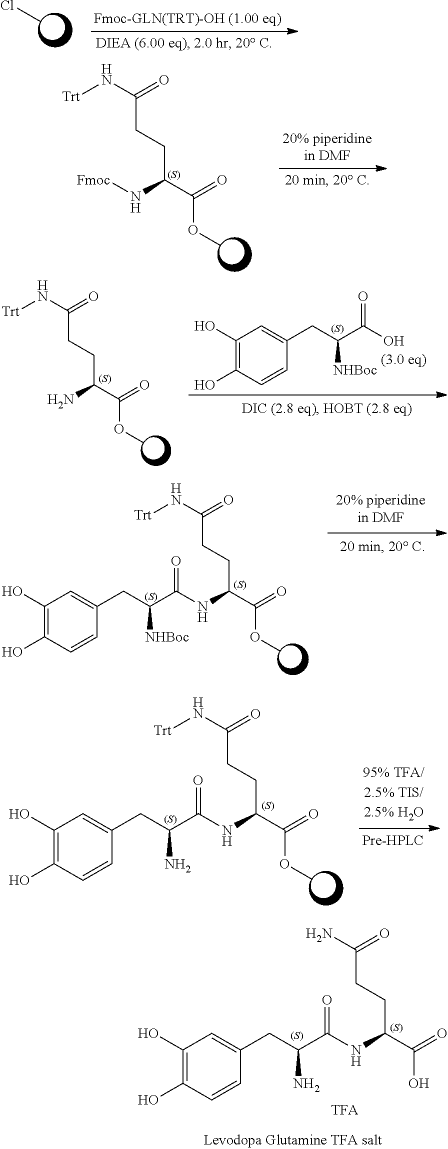

### Preparation of Levodopa Asparagine TFA Salt (LD-Asn TFA Salt)

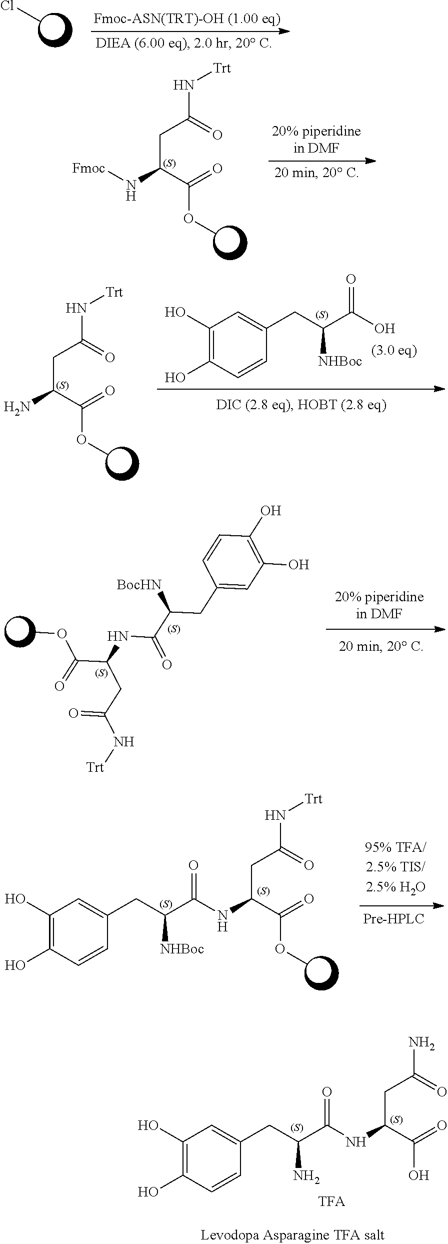

### Preparation of Levodopa Tyrosine TFA Salt (LD-Tyr TFA Salt)

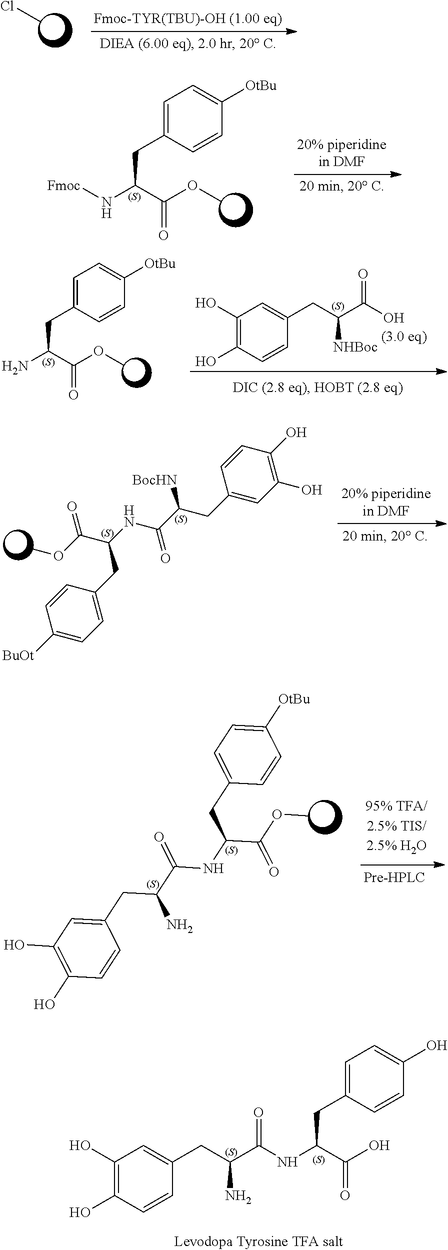

### Preparation of Levodopa Tryptophan (LD-Trp)

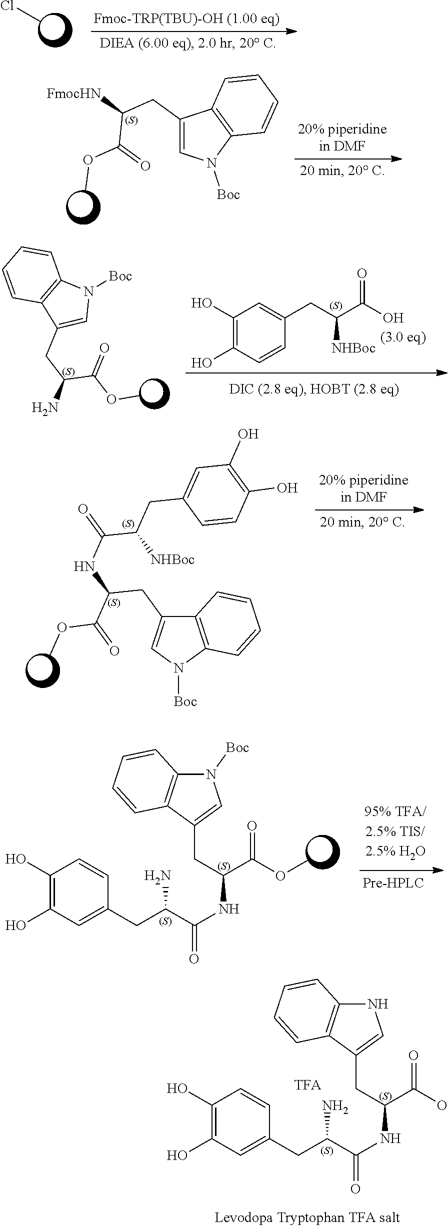

### Preparation of Levodopa-Lanthionine TFA Salt (LD-LA TFA Salt)

### Step 1: Halogenation

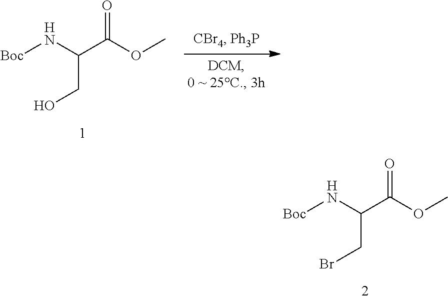

### Step 2: Hydrolysis

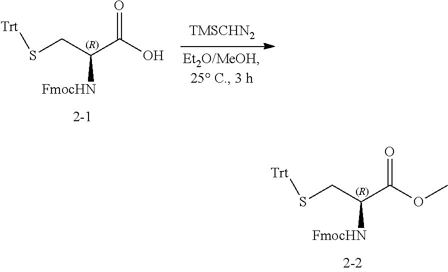

### Step 3: Deprotection

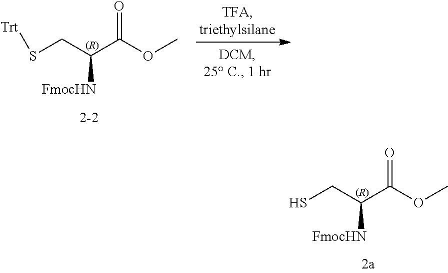

### Step 4: Coupling

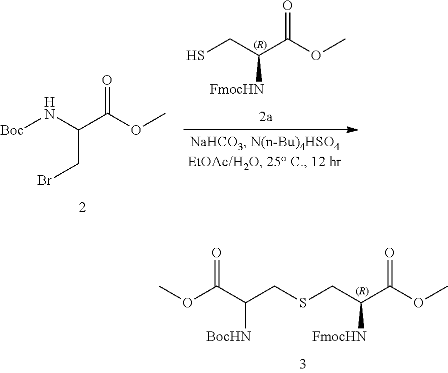

Step 5: Coupling with Protected Levodopa

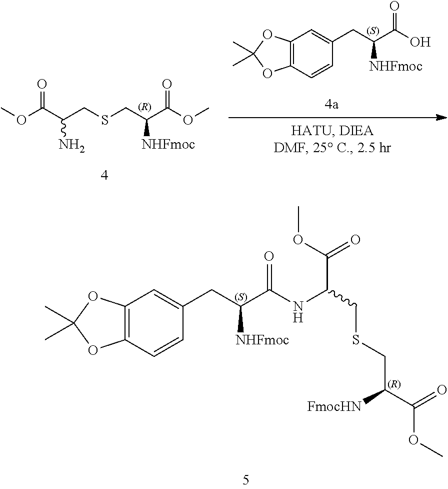

### Step 6: Deprotection (Fmoc Removal) and Diastereomers Separation

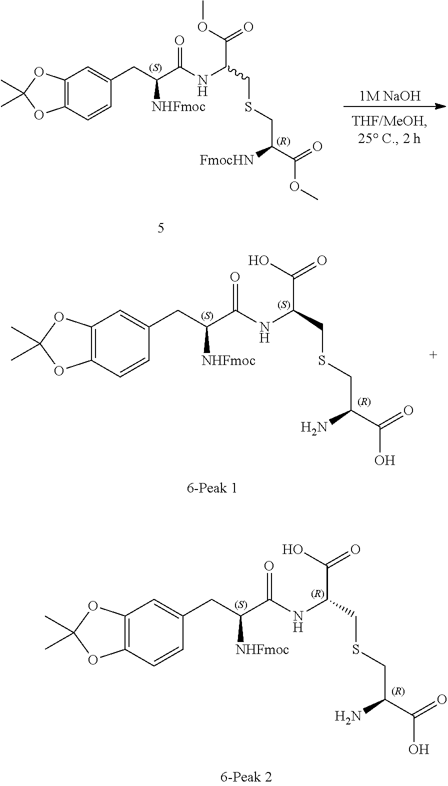

### Step 7a: Deprotection of LD and Isolation of Levodopa Lanthionine Peak 1 TFA Salt (LD-La 1 TFA Salt)

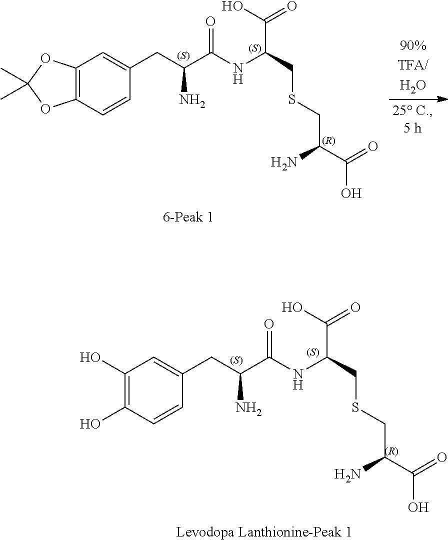

### Step 7b: Deprotection of LD and Isolation of Levodopa Lanthionine Peak 2 TFA Salt (LD-La 2 TFA Salt)

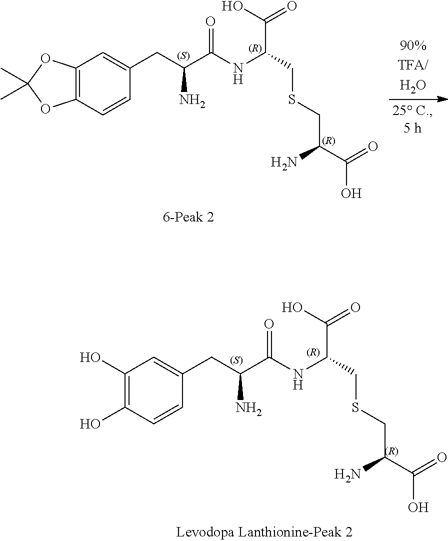

It is noted that, throughout this document, although LD-LA 1 (referred to also as levodopa lanthionine peak 1 or levodopa lanthionine 1) is demonstrated as being the (S)(S)(R) isomer, and LD-LA 2 (referred to also as levodopa lanthionine peak 1 or levodopa lanthionine 1) is demonstrated as being the (S)(R)(R) isomer, the two prepared isomers were not fully identified and therefore, the isomers may be opposite to what is demonstrated and depicted throughout this document.

### Example 2—Preparation of Levodopa Amino Acids (LDAA) Free Base Forms

### CBz Protection of L-DOPA

The synthesis was performed using CBz-chloride and NaOH as the base. L-DOPA (200 g, 1.014 moL) was suspended in water (600 mL) and cooled to 0° C. under nitrogen. A mixture of NaOH (81.3 g, 2.033 mol) in water (600 mL) was added at 0° C. CBz-chloride (211.4 g, 1.239 mol) in dioxane (800 mL) was added at 0° C. over the course of 1 h. The mixture was allowed to warm to room temperature. After approximately 1 hour, a conversion of 73% was observed. Another portion of NaOH (4.9 g, 0.123 mol) in water (60 mL) and CBz-chloride (20.8 g, 0.122 mol) in dioxane (80 mL) was added. The reaction mixture was stirred overnight at room temperature. A conversion of 83% was observed. Another portion of NaOH (8.1 g, 0.203 mol) in water (50 mL) and CBz-chloride (35 g, 0.205 mol) in dioxane (50 mL) was added. When a conversion of 94% was obtained (1.5 h after addition) the pH was adjusted to 10 with 3 M NaOH, and the mixture was washed with MTBE (1 L). The pH of the aqueous phase was adjusted to 2 using 6 M HCl, and the aqueous phase was extracted with MTBE (2×1 L). The combined organic phases were washed with water (1 L) and 25% NaCl, aq. (1 L). The organic phase was dried over sodium sulphate, filtered, evaporated under reduced pressure and dried in vacuum to give 448.3 g (135%) as a sticky, brown mass (purity (280 nm) was 82.5%).

### Deprotection of CBz-L-DOPA-(CBz/Bn)-Lys

CBz-L-DOPA-(CBz/Bn)-Lys (93.4 g) was dissolved in methanol (4.2 L). The atmosphere was exchanged for nitrogen (3 times), after which 10% Pd/C (18.8 g) was added and the atmosphere was again exchanged for nitrogen (2 times) and, subsequently, hydrogen (3 times). The reactor was evacuated/filled with hydrogen. After 4.5 h it was estimated by HPLC analysis that the reaction was complete. The reaction mixture was filtered through Celite® and evaporated under reduced pressure at a water bath temperature of 40° C. The compound precipitated during evaporation. When approximately 400 mL was left, the suspension was filtered, and the filter cake was washed with methanol (50 mL). The solid was dried in vacuum at 25° C. overnight to provide 33.1 g (75%) of LD-Lys free base as an off-white solid (purity was 99.0%).

### Example 2.2—Preparation of the Free Base Form of LD-Tyr Coupling with H-Tyr-OBzl

EDC-Cl (46.3 g, 242 mmol) was added in portions, over the course of 10 min, to a solution of BnO-Tyr (64.9 g, 239 mmol), HOBt (36.8 g, 88 w/w %, 240 mmol) and CBz-L-DOPA (363.1 g, 20.1 w/w % solution in DMF, 220 mmol) in DMF (863.2 g, 0.9 L), at 0° C. The reaction was stirred at 0° C. for 4 hours before water (1.7 kg) was added over the course of 30 min, and the reaction mixture was allowed to heat to ambient temperature. EtOAc (2.6 kg, 2.8 L) was charged to the reactor and the phases separated. The organic phase was washed with water three times (1.5 L, 1.4 L and 1.4 L). Celite® (450 g) was added to the crude organic phase, and the mixture was concentrated to dryness. The crude residue was purified by flash column chromatography (silica gel column, 3.2 kg packed with EtOAc/dichloromethane 1:1 (v/v)), by loading the Celite®-mixture onto the column and eluting with EtOAc/dichloromethane 1:1 (v/v). Selected fractions (12 L) were concentrated under reduced pressure at a water bath having a temperature of 45° C. The selected fractions were further dried in vacuum overnight. L-DOPA-BnOTyr was isolated as a slightly brown solid (44.7 g, 35%) with a purity of 96.4%. The column was flushed with 20% MeOH in CH₂Cl₂ (10 L) and all fractions containing L-DOPA-Bn-OTyr were collected and concentrated under reduced pressure at a water bath with temperature of 45° C. The crude residue (60.2 g) was dissolved in 2-PrOH (432.1 g, 550 mL) by heating the mixture to 75° C. The solution was filtered while hot and allowed to cool to ambient temperature and stirred overnight to give a precipitate. The suspension was filtered, and the filter cake washed with 2-PrOH (166 g, 211 mL) and dried in vacuum at 30° C. overnight to yield CBz-L-DOPA-Tyr(OBn) as a white solid (34.9 g, 27%) with a purity of 95.1%.

### Deprotection of CBz-L-DOPA-Tyr(OBn)

A solution of L-DOPA-BnOTyr (60.4 g, 103 mmol) in MeOH (2051 g, 2.6 L) was purged with nitrogen three times (vacuum (<250 mbar) followed by filling with N₂). 10% Pd/C (12.0 g) was added to the reactor, which was subsequently purged with hydrogen (vacuum (>250 mbar) followed by filling with H₂). The reactor was evacuated/filled with H₂ after 1 hour and 20 minutes and left an additional 30 min before being evacuated/filled with N₂ and filtered through Celite®. The filter cake was washed with MeOH (418.9 g, 529 mL), and the combined filtrates were concentrated under reduced pressure. At approximately a volume of 500 mL the solution was filtered through a 0.45 μm pore filter and the filtrate concentrated to dryness under reduced pressure. The oily solid was dried overnight at vacuum to yield LD-Tyr free base as an off-white solid (36.5 g, 98%) with a purity of 95.4%.

### Example 3—Synthesis of LD-Lys HCl, LD-Tyr HCl and LD-Arg HCl Salts

### Example 3.1—Synthesis of LD-Arg HCl Salt—Method #1

Coupling with H-Arginine(NO₂)-OBn

CBz-L-DOPA (342.9 g, 20.1 w/w % solution in DMF, 208 mmol) was dissolved in DMF (690 mL). HOBt.H₂O (35.2 g, 228 mmol (88% w/w)) and H-Arg(NO₂)OBn, p-tosylate (110.0 g, 228 mmol) were added. The solution was cooled to 0° C. Triethylamine (23.2 g, 228 mmol) was added, and then EDC. HCl (43.7 g, 228 mmol) was added in portions, while the temperature was kept at 0° C. The coupling mixture was stirred for 2.5 h and then quenched with water (1400 mL). The mixture was extracted with EtOAc three times (1400 mL and 2×700 mL). The organic phases were combined.

The organic phase was evaporated under reduced pressure at a water bath temperature of 40° C. The residue was dissolved in 8 vol distilled THF, 8 volumes water was added, resulting in an emulsion. The emulsion was applied to a reverse phase column (26 equivalents of Phenomenex Sepra C-18-T (50 μm, 135 Å) packed with THF and conditioned with 700 mL 20% distilled THF/water). The column was eluted with 40% distilled THF in water. The pure fractions were evaporated under reduced pressure until mainly water was left. The suspension was cooled and filtered. The filter cake was dried to provide a solid (259 g) that was not dried; rather, it was placed in a freezer until further processing.

### CBz-L-DOPA-Arg(NO2)-(OBn)

CBz-L-DOPA-Arg(NO2)-(OBn) (230.4 g wet, approximately 68.6 g dry, 110 mmol) was suspended in methanol (6.45 L) and water (1.29 L), and HCl (36%, aq., 43 mL) was added. The reaction flask was evacuated to 250 mbar, and the atmosphere was exchanged for nitrogen three times. The mixture was heated to 40° C.

10% Pd/C (14.0 g) was added and the atmosphere was exchanged for nitrogen (3 times) and then hydrogen (3 times). The reaction mixture was protected from light. The atmosphere was exchanged for hydrogen. After 3 h, the atmosphere was exchanged for nitrogen (3 times). The suspension was filtered through Celite®, and the filter cake was washed with 20% water/methanol (600 mL). The pH of the filtrate was adjusted to pH 6 using an ion exchange resin (Dowex 1x8 chloride form, pre-activated with 1 M NaOH and washed with water to pH 7). The pH was adjusted in four portions, each of which was filtered and washed with 20% water/methanol (250 mL). The filtrates were evaporated under reduced pressure at a water bath temperature of 50° C. to a volume of approximately 500 mL. The residue was treated with activated carbon (5.0 g) for 40 minutes. The suspension was filtered over Celite®, the filter cake was washed with water (150 mL), and the combined filtrate and wash were concentrated to dryness under reduced pressure at a water bath temperature of 50° C. The solid residue was dried overnight in vacuum to provide 48.1 g as a light brown solid (purity 95.6%). The prepared LD-Arg HCl salt comprises one equivalent of HCl.

### Example 3.2—Synthesis of LD-Arg HCl Salt—Method #2

### Synthesis of N-Boc-L-Dopa

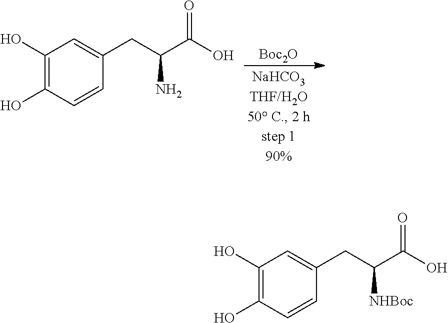 [SMILES: "Boc2O" ; N[C@@H](Cc1ccc(O)c(O)c1)C(=O)O ; CC(C)(C)OC(=O)N[C@@H](Cc1ccc(O)c(O)c1)C(=O)O]

### Synthesis of 2,5-dioxopyrrolidin-1-yl (2S)-2-[(tert-butoxycarbonyl) amino]-3-(3,4-dihydroxyphenyl) propanoate

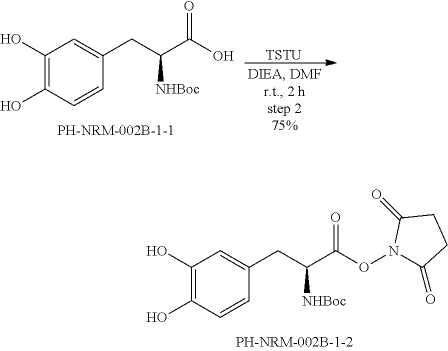

### Synthesis of (2S)-2-[(2S)-2-[(tert-butoxycarbonyl)amino]-3-(3,4-dihydroxyphenyl)propanamido]-5-carbamimidamidopentanoic acid

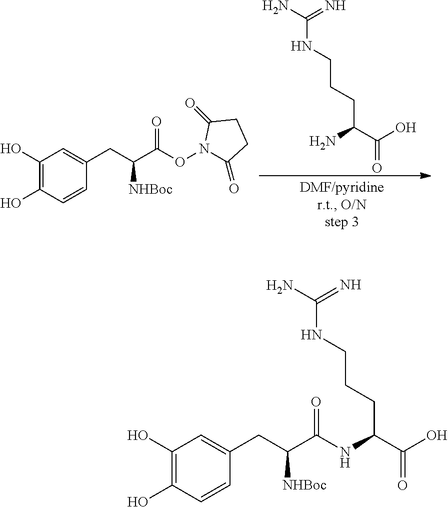 [SMILES: CC(C)(C)OC(=O)N[C@@H](Cc1ccc(O)c(O)c1)C(=O)ON1C(=O)CCC1=O ; N=C(N)NCCC[C@H](N)C(=O)O ; CC(C)(C)OC(=O)N[C@@H](Cc1ccc(O)c(O)c1)C(=O)N[C@@H](CCCNC(=N)N)C(=O)O]

### Synthesis of LD-Arg HCl

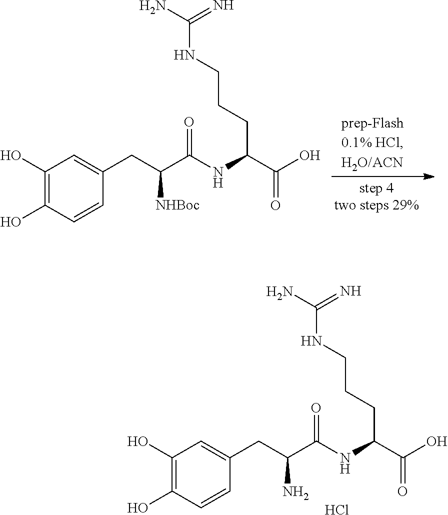 [SMILES: CC(C)(C)OC(=O)N[C@@H](Cc1ccc(O)c(O)c1)C(=O)N[C@@H](CCCNC(=N)N)C(=O)O ; N=C(N)NCCC[C@H](NC(=O)[C@@H](N)Cc1ccc(O)c(O)c1)C(=O)O ; Cl]

### Example 3.2—Synthesis of LD-Tyr HCl Salt

### Synthesis of benzyl (2S)-2-[(2S)-2-[(tert-butoxycarbonyl)amino]-3-(3,4-dihydroxyphenyl)propanamido]-3-(4-hydroxyphenyl)propanoate

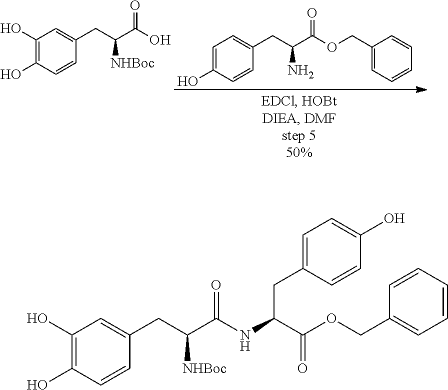 [SMILES: CC(C)(C)OC(=O)N[C@@H](Cc1ccc(O)c(O)c1)C(=O)O ; N[C@@H](Cc1ccc(O)cc1)C(=O)OCc1ccccc1 ; CC(C)(C)OC(=O)N[C@@H](Cc1ccc(O)c(O)c1)C(=O)N[C@@H](Cc1ccc(O)cc1)C(=O)OCc1ccccc1]

### Synthesis of (2S)-2-[(2S)-2-[(tert-butoxycarbonyl)amino]-3-(3,4-dihydroxyphenyl)propanamido]3-(4-hydroxyphenyl) propanoic acid

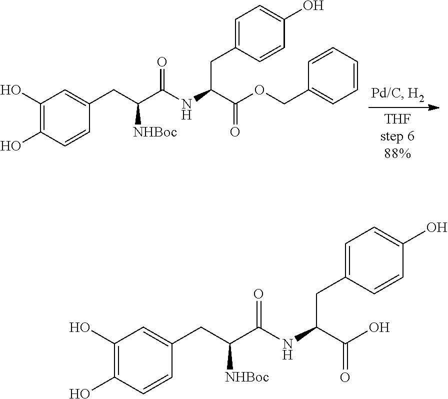 [SMILES: CC(C)(C)OC(=O)N[C@@H](Cc1ccc(O)c(O)c1)C(=O)N[C@@H](Cc1ccc(O)cc1)C(=O)OCc1ccccc1 ; CC(C)(C)OC(=O)N[C@@H](Cc1ccc(O)c(O)c1)C(=O)N[C@@H](Cc1ccc(O)cc1)C(=O)O]

### Synthesis of LD-Tyr HCl

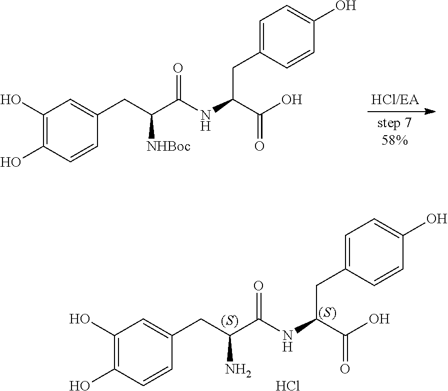

### Example 3.3—Synthesis of LD-Lys HCl Salt

### Synthesis of benzyl (2S)-6-[(tert-butoxycarbonybamino]-2-[(2S)-2-[(tert-butoxycarbonyl)amino]-3-(3,4-dihydroxyphenyl)propanamido]hexanoate

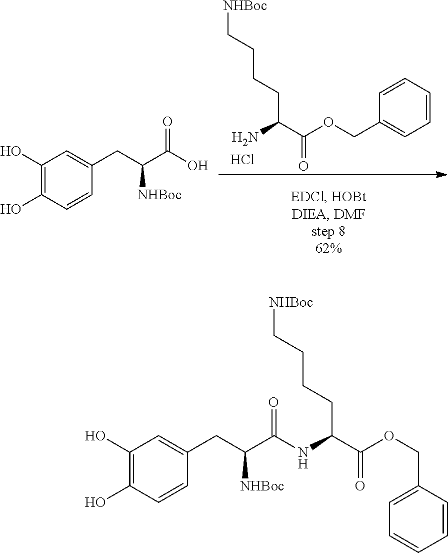 [SMILES: CC(C)(C)OC(=O)N[C@@H](Cc1ccc(O)c(O)c1)C(=O)O ; CC(C)(C)OC(=O)NCCCC[C@H](N)C(=O)OCc1ccccc1 ; Cl ; CC(C)(C)OC(=O)NCCCC[C@H](NC(=O)[C@H](Cc1ccc(O)c(O)c1)NC(=O)OC(C)(C)C)C(=O)OCc1ccccc1]

### Synthesis of (2S)-6-[(tert-butoxycarbonyl)amino]-2-[(2S)-2-[(tert-butoxycarbonyl)amino]-3-(3,4-dihydroxyphenyl)propanamido]hexanoic acid

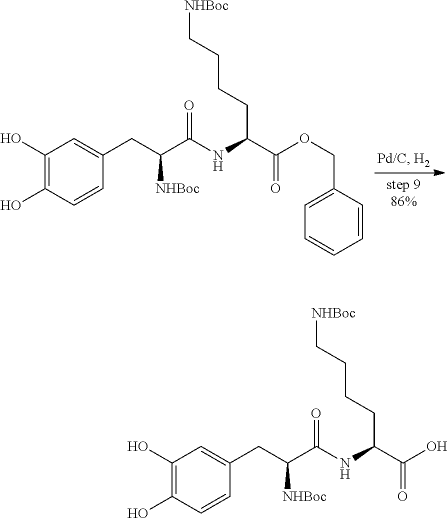 [SMILES: CC(C)(C)OC(=O)NCCCC[C@H](NC(=O)[C@H](Cc1ccc(O)c(O)c1)NC(=O)OC(C)(C)C)C(=O)OCc1ccccc1 ; CC(C)(C)OC(=O)NCCCC[C@H](NC(=O)[C@H](Cc1ccc(O)c(O)c1)NC(=O)OC(C)(C)C)C(=O)O]

### Synthesis of LD-Lys HCl

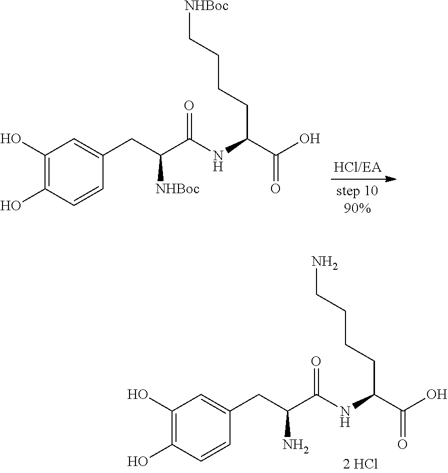 [SMILES: CC(C)(C)OC(=O)NCCCC[C@H](NC(=O)[C@H](Cc1ccc(O)c(O)c1)NC(=O)OC(C)(C)C)C(=O)O ; NCCCC[C@H](NC(=O)[C@@H](N)Cc1ccc(O)c(O)c1)C(=O)O]

### Example 4 Production of (2S)-6-amino-2-[[(2S)-2-amino-3-(3-hydroxy-4-phosphonooxyphenyl)propanoyl]amino]hexanoic acid

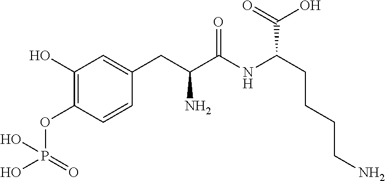 [SMILES: NCCCC[C@H](NC(=O)[C@@H](N)Cc1ccc(OP(=O)(O)O)c(O)c1)C(=O)O]

### Examples 5-18

The corresponding starting compounds were respectively treated in a similar manner as in Example 4 to obtain the compounds shown in Table 2 below.

TABLE 2

| Example | Structural formula | Physical property values |
|---|---|---|
| 5 | 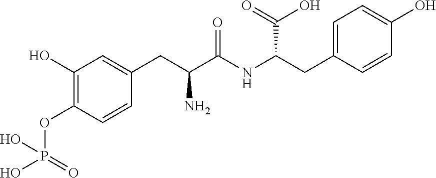 [SMILES: N[C@@H](Cc1ccc(OP(=O)(O)O)c(O)c1)C(=O)N[C@@H](Cc1ccc(O)cc1)C(=O)O] | MS (ESI); m/z 441.4 [M + H]+ |
| 6 | 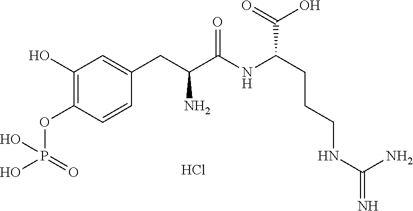 [SMILES: N=C(N)NCCC[C@H](NC(=O)[C@@H](N)Cc1ccc(OP(=O)(O)O)c(O)c1)C(=O)O ; Cl] | MS (ESI); m/z 434.4 [M + H]+ |
| 7 | 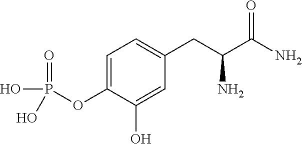 [SMILES: NC(=O)[C@@H](N)Cc1ccc(OP(=O)(O)O)c(O)c1] | MS (ESI); m/z 277.2 [M + H]+ |
| 8 | 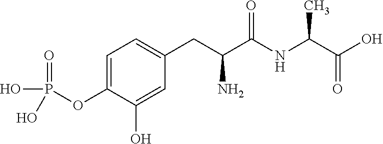 [SMILES: C[C@H](NC(=O)[C@@H](N)Cc1ccc(OP(=O)(O)O)c(O)c1)C(=O)O] | MS (ESI); m/z 349.2 [M + H]+ |
| 9 | 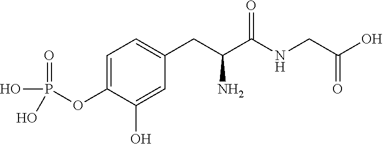 [SMILES: N[C@@H](Cc1ccc(OP(=O)(O)O)c(O)c1)C(=O)NCC(=O)O] | MS (ESI); m/z 335.2 [M + H]+ |
| 10 | 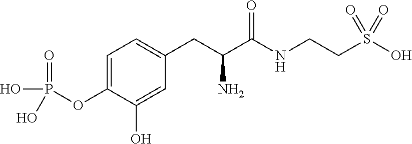 [SMILES: N[C@@H](Cc1ccc(OP(=O)(O)O)c(O)c1)C(=O)NCCS(=O)(=O)O] | MS (ESI); m/z 383.1 [M − H]− |
| 11 | 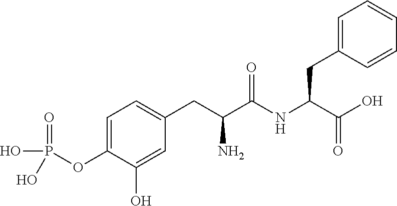 [SMILES: N[C@@H](Cc1ccc(OP(=O)(O)O)c(O)c1)C(=O)N[C@@H](Cc1ccccc1)C(=O)O] | MS (ESI); m/z 425.4 [M + H]+ |
| 12 | 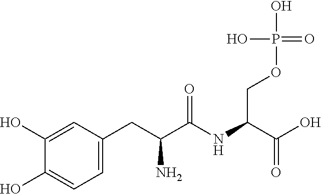 [SMILES: N[C@@H](Cc1ccc(O)c(O)c1)C(=O)N[C@@H](COP(=O)(O)O)C(=O)O] | MS (ESI); m/z 365.2 [M + H]+ |
| 13 | 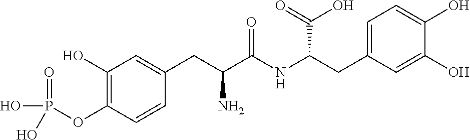 [SMILES: N[C@@H](Cc1ccc(OP(=O)(O)O)c(O)c1)C(=O)N[C@@H](Cc1ccc(O)c(O)c1)C(=O)O] | MS (ESI); m/z 457.1 [M + H]+ |
| 14 | 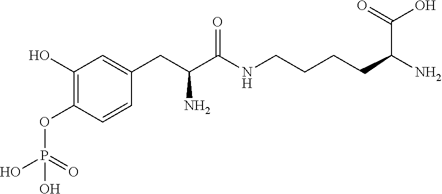 [SMILES: N[C@@H](CCCCNC(=O)[C@@H](N)Cc1ccc(OP(=O)(O)O)c(O)c1)C(=O)O] | MS (ESI); m/z 406.2 [M + H]+ |
| 15 | 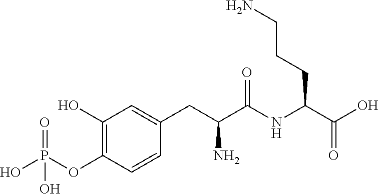 [SMILES: NCCC[C@H](NC(=O)[C@@H](N)Cc1ccc(OP(=O)(O)O)c(O)c1)C(=O)O] | MS (ESI); m/z 392.2 [M + H]+ |
| 16 | 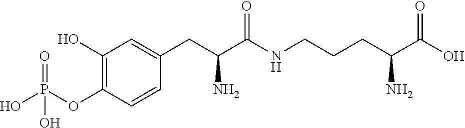 [SMILES: N[C@@H](CCCNC(=O)[C@@H](N)Cc1ccc(OP(=O)(O)O)c(O)c1)C(=O)O] | MS (ESI); m/z 392.0 [M + H]+ |
| 17 | 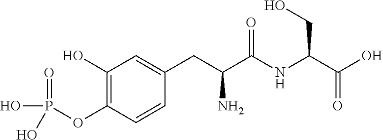 [SMILES: N[C@@H](Cc1ccc(OP(=O)(O)O)c(O)c1)C(=O)N[C@@H](CO)C(=O)O] | MS (ESI); m/z 365.1 [M + H]+ |
| 18 | 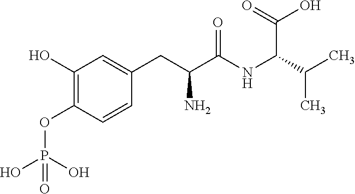 [SMILES: CC(C)[C@H](NC(=O)[C@@H](N)Cc1ccc(OP(=O)(O)O)c(O)c1)C(=O)O] | MS (ESI); m/z 377.1 [M + H]+ |

### Example 19—Production of (2S)-2-[[(2S)-2-amino-3-(3-hydroxy-4-phosphonooxyphenyl)propanoyl]amino]-3-(3-hydroxy-4-phosphonooxyphenyl)propanoic acid

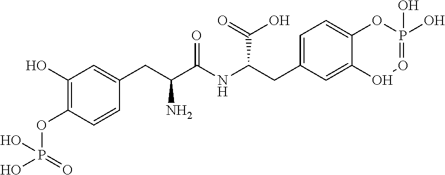

(1) A suspension of dibenzyl N,N-diisopropyl phosphoramidite (615 uL) and 1H-tetrazole (115 mg) in acetonitrile (3 mL) was added to a solution of benzyl (2S)-3-(4-hydroxy-3-phenylmethoxyphenyl)-2-[[(2S)-3-(4-hydroxy-3-phenylmethoxyphenyl)-2-(phenylmethoxycarbonylamino)propanoyl]amino]propanoate (430 mg) in dichloromethane (9 mL), and the mixture was stirred at room temperature for 13.5 hours. Dibenzyl N,N-diisopropyl phosphoramidite (205 uL) and 1H-tetrazole (35 mg) were added, and the mixture was stirred at room temperature for 1 hour. The reaction mixture was ice-cooled, a TERT-butyl hydroperoxide aqueous solution (70%) (0.39 mL) was added, and the mixture was stirred at room temperature for 1 hour. The reaction mixture was diluted with chloroform, an organic layer was washed with a saturated aqueous sodium hydrogen carbonate solution and a saturated solution of sodium chloride, and was dried over sodium sulfate, and then, the solvent was distilled away under a reduced pressure. The obtained residue is purified by silica gel column chromatography (solvent: hexane/(ethyl acetate)=60/40-35/65), and thereby, a crude product of benzyl (2S)-3-[4-bis(phenylmethoxy)phosphoryloxy-3-phenylmethoxyphenyl]-2-[[(2S)-3-[4-bis(phenylmethoxy)phosphoryloxy-3-phenylmethoxy phenyl]-2-(phenylmethoxycarbonylamino)propanoyl]amino]propanoate (565 mg) was obtained.

(2) The crude product of benzyl (2S)-3-[4-bis(phenylmethoxy)phosphoryloxy-3-phenylmethoxyphenyl]-2-[[(2S)-3-[4-bis(phenylmethoxy)phosphoryloxy-3-phenylmethoxy phenyl]-2-(phenylmethoxycarbonylamino)propanoyl]amino]propanoate (290 mg) was dissolved in a mixed solvent of tetrahydrofuran (4 mL), acetic acid (1 mL) and water (0.5 mL), and palladium/carbon (wet) (50 mg) was added, and the mixture was stirred under a hydrogen atmosphere at room temperature for 25.5 hours. Water (10 mL) was added, and the mixture was stirred under a hydrogen atmosphere at room temperature for 1.5 hours. The reaction mixture was filtered through a membrane filter (cellulose acetate) to remove insoluble matter. The insoluble matter was washed with water (20 mL). After freeze-drying, the title compound (112 mg) was obtained.

MS(ESI); m/z 537.3 [M+H]+

### Examples 20 and 21

The corresponding starting compounds were respectively treated in a similar manner as in Example 4 to obtain the compounds shown in Table 3 below.

TABLE 3

| Example | Structural formula | Physical property values |
|---|---|---|
| 20 | 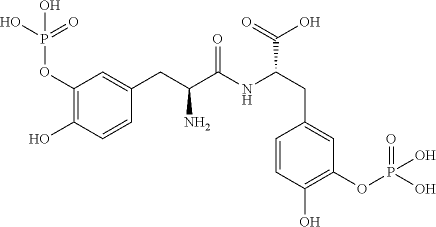 [SMILES: N[C@@H](Cc1ccc(O)c(OP(=O)(O)O)c1)C(=O)N[C@@H](Cc1ccc(O)c(OP(=O)(O)O)c1)C(=O)O] | MS (ESI); m/z 537.3 [M + H]+ |
| 21 | 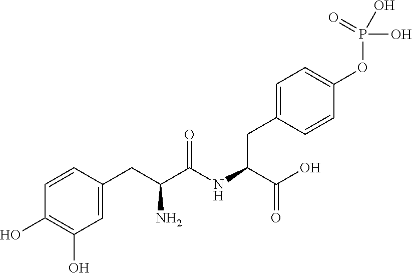 [SMILES: N[C@@H](Cc1ccc(O)c(O)c1)C(=O)N[C@@H](Cc1ccc(OP(=O)(O)O)cc1)C(=O)O] | MS (ESI); m/z 441.3 [M + H]+ |

Reference Example 1—Production of benzyl (2S)-2-[[(2S)-3-[4-bis(phenylmethoxy)phosphoryloxy-3-phenylmethoxyphenyl]-2-(phenylmethoxycarbonylamino)propanoyl]amino]-6-(phenylmethoxycarbonylamino)hexanoato

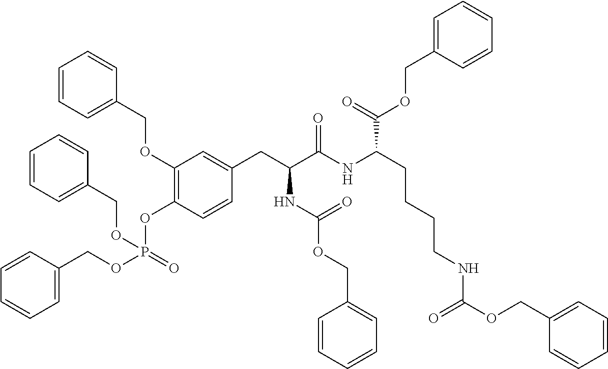 [SMILES: O=C(NCCCC[C@H](NC(=O)[C@H](Cc1ccc(OP(=O)(OCc2ccccc2)OCc2ccccc2)c(OCc2ccccc2)c1)NC(=O)OCc1ccccc1)C(=O)OCc1ccccc1)OCc1ccccc1]

Dibenzyl N,N-diisopropyl phosphoramidite (3.28 mL) and 1H-tetrazole (0.62 g) were added to a suspension of benzyl (2S)-2-[[(2S)-3-(4-hydroxy-3-phenylmethoxyphenyl)-2-(phenylmethoxycarbonylamino)propanoyl]amino]-6-(phenylmethoxycarbonylamino)hexanoato (4.57 g) in dichloromethane (45 mL) and acetonitrile (18 mL) under ice cooling, and the mixture was stirred at room temperature for 1 hour. The reaction mixture was ice-cooled, a TERT-butyl hydroperoxide aqueous solution (70%) (1.2 mL) was added, and the mixture was stirred at room temperature for 19 hours. The solvent of the reaction mixture was distilled away under a reduced pressure, a saturated aqueous sodium hydrogen carbonate solution and water were added, and extraction with ethyl acetate was performed. An organic layer was distilled away under a reduced pressure. The obtained residue was purified by silica gel column chromatography (solvent: hexane/(ethyl acetate)=67/33-40/60), and thereby, the title compound (5.38 g, 85%) as a white powder was obtained.

MS(ESI); m/z 1034.4[M+H]+

### Reference Examples 2-13

The corresponding starting compounds were respectively treated in a similar manner as in Reference Example 1 to obtain the compounds shown in Table 4 below.

TABLE 4

| Reference | | Physical property |
|---|---|---|
| Example | Structural formula | values and the like |
| 2 | 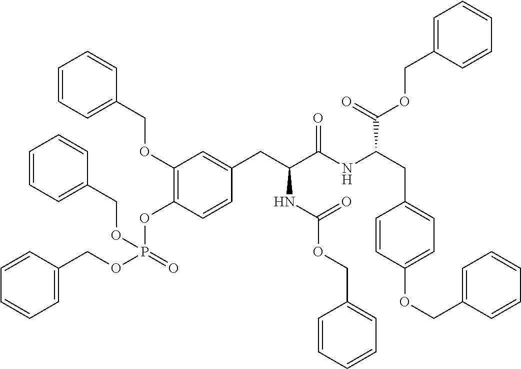 [SMILES: O=C(N[C@@H](Cc1ccc(OP(=O)(OCc2ccccc2)OCc2ccccc2)c(OCc2ccccc2)c1)C(=O)N[C@@H](Cc1ccc(OCc2ccccc2)cc1)C(=O)OCc1ccccc1)OCc1ccccc1] | MS (ESI); m/z 1025.6 [M + H]+ |
| 3 | 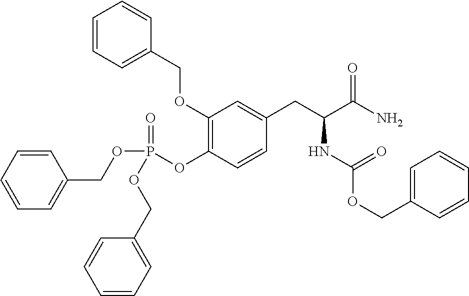 [SMILES: NC(=O)[C@H](Cc1ccc(OP(=O)(OCc2ccccc2)OCc2ccccc2)c(OCc2ccccc2)c1)NC(=O)OCc1ccccc1] | MS (ESI); m/z 681.4 [M + H]+ |
| 4 | 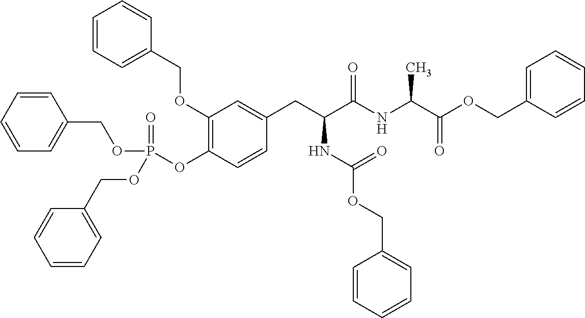 [SMILES: C[C@H](NC(=O)[C@H](Cc1ccc(OP(=O)(OCc2ccccc2)OCc2ccccc2)c(OCc2ccccc2)c1)NC(=O)OCc1ccccc1)C(=O)OCc1ccccc1] | MS (ESI); m/z 843.4 [M + H]+ |
| 5 | 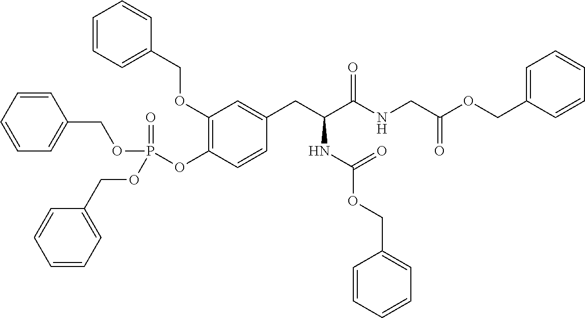 [SMILES: O=C(CNC(=O)[C@H](Cc1ccc(OP(=O)(OCc2ccccc2)OCc2ccccc2)c(OCc2ccccc2)c1)NC(=O)OCc1ccccc1)OCc1ccccc1] | MS (ESI); m/z 829.4 [M + H]+ |
| 6 | 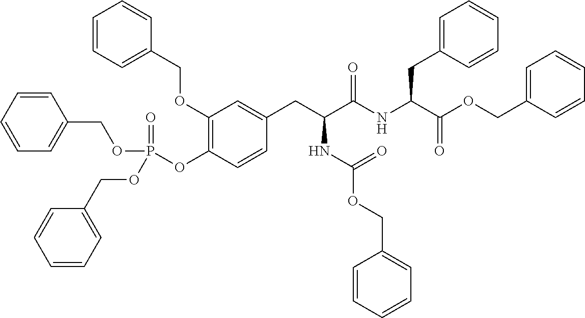 [SMILES: O=C(N[C@@H](Cc1ccc(OP(=O)(OCc2ccccc2)OCc2ccccc2)c(OCc2ccccc2)c1)C(=O)N[C@@H](Cc1ccccc1)C(=O)OCc1ccccc1)OCc1ccccc1] | MS (ESI); m/z 919.5 [M + H]+ |
| 7 | 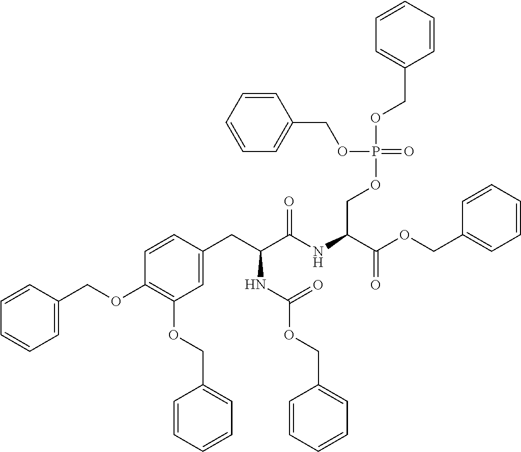 [SMILES: O=C(N[C@@H](Cc1ccc(OCc2ccccc2)c(OCc2ccccc2)c1)C(=O)N[C@@H](COP(=O)(OCc1ccccc1)OCc1ccccc1)C(=O)OCc1ccccc1)OCc1ccccc1] | MS (ESI); m/z 949.5 [M + H]+ |
| 8 | 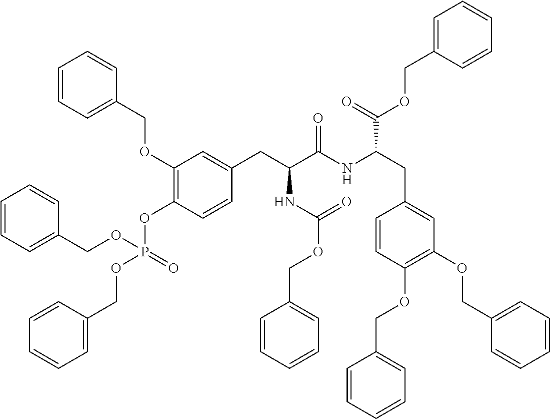 [SMILES: O=C(N[C@@H](Cc1ccc(OP(=O)(OCc2ccccc2)OCc2ccccc2)c(OCc2ccccc2)c1)C(=O)N[C@@H](Cc1ccc(OCc2ccccc2)c(OCc2ccccc2)c1)C(=O)OCc1ccccc1)OCc1ccccc1] | MS (ESI); m/z 1129.5 [M − H]− |
| 9 | 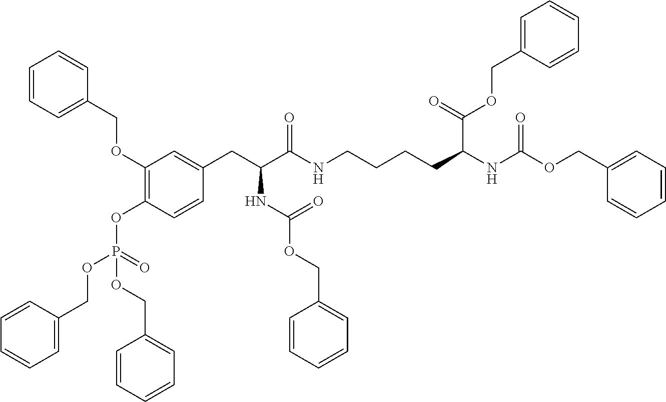 [SMILES: O=C(N[C@@H](Cc1ccc(OP(=O)(OCc2ccccc2)OCc2ccccc2)c(OCc2ccccc2)c1)C(=O)NCCCC[C@H](NC(=O)OCc1ccccc1)C(=O)OCc1ccccc1)OCc1ccccc1] | MS (ESI); m/z 1051.8 [M + H + NH3]+ |
| 10 | 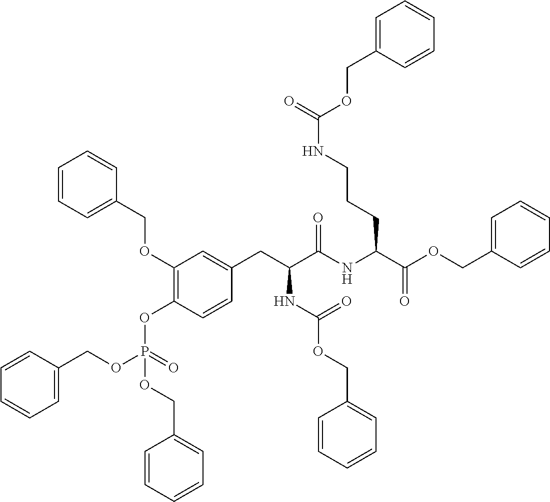 [SMILES: O=C(NCCC[C@H](NC(=O)[C@H](Cc1ccc(OP(=O)(OCc2ccccc2)OCc2ccccc2)c(OCc2ccccc2)c1)NC(=O)OCc1ccccc1)C(=O)OCc1ccccc1)OCc1ccccc1] | MS (ESI); m/z 1018.4 [M − H]− |
| 11 | 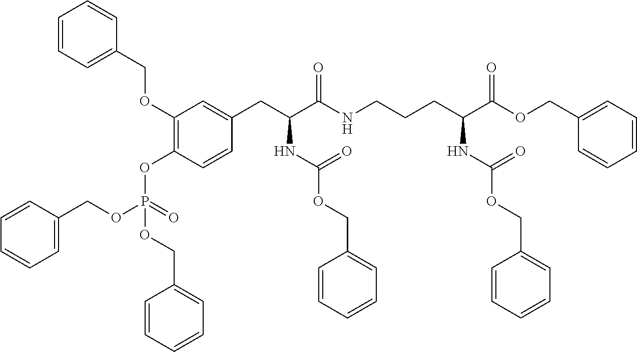 [SMILES: O=C(N[C@@H](Cc1ccc(OP(=O)(OCc2ccccc2)OCc2ccccc2)c(OCc2ccccc2)c1)C(=O)NCCC[C@H](NC(=O)OCc1ccccc1)C(=O)OCc1ccccc1)OCc1ccccc1] | MS (ESI); m/z 1018.4 [M − H]− |
| 12 | 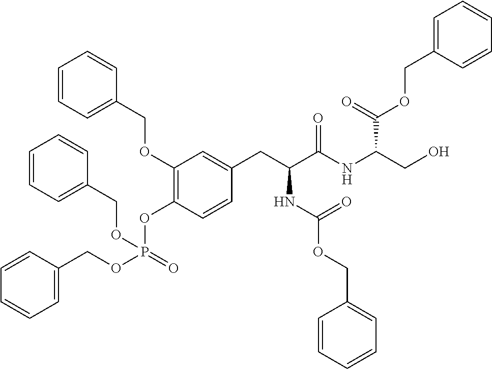 [SMILES: O=C(N[C@@H](Cc1ccc(OP(=O)(OCc2ccccc2)OCc2ccccc2)c(OCc2ccccc2)c1)C(=O)N[C@@H](CO)C(=O)OCc1ccccc1)OCc1ccccc1] | MS (ESI); m/z 857.3 [M − H]+ |
| 13 | 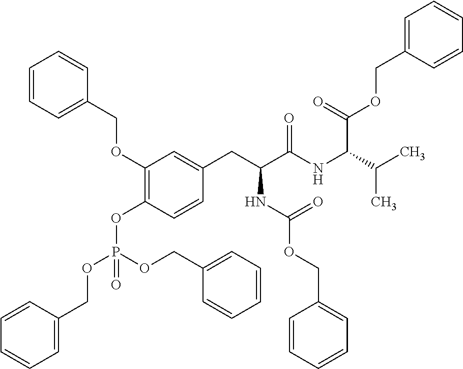 [SMILES: CC(C)[C@H](NC(=O)[C@H](Cc1ccc(OP(=O)(OCc2ccccc2)OCc2ccccc2)c(OCc2ccccc2)c1)NC(=O)OCc1ccccc1)C(=O)OCc1ccccc1] | MS (ESI); m/z 869.6 [M − H]− |

### Reference Example 2′—Production of Benzyl (2S)-2-[[(2S)-3-[4-bis(phenylmethoxy)phosphoryloxy-3-phenylmethoxyphenyl]-2-(phenylmethoxycarbonylamino)propanoyl]amino]-3-(4-phenylmethoxyphenyl)propanoate

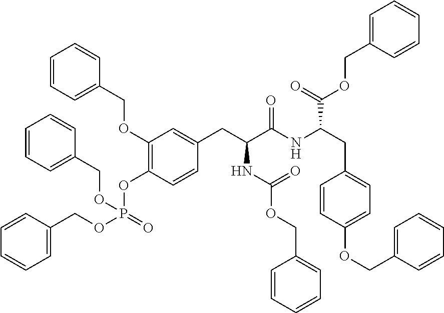 [SMILES: O=C(N[C@@H](Cc1ccc(OP(=O)(OCc2ccccc2)OCc2ccccc2)c(OCc2ccccc2)c1)C(=O)N[C@@H](Cc1ccc(OCc2ccccc2)cc1)C(=O)OCc1ccccc1)OCc1ccccc1]

Benzyl(2S)-2-amino-3-(4-benzyloxyphenyl)propanoic acid; hydrochloride (277 mg), N,N-diisopropylethylamine (0.35 mL), 1-ethyl-3-(3-dimethylaminopropyl)carbodiimide hydrochloride (WSCI) (115 mg) and 1-hydroxy-7-azabenzotriazole (HOAt) (82 mg) were added to a mixture of (2S)-3-[4-bis(phenylmethoxy)phosphoryloxy-3-phenylmethoxyphenyl]-2-(phenylmethoxycarbonylamino)propanoic acid (352 mg) and N,N-dimethylformamide (4 mL), and the mixture was stirred at room temperature for 16 hours. An organic layer was washed with water and a saturated solution of sodium chloride, and was dried over sodium sulfate, and the solvent was distilled away under a reduced pressure. The obtained residue was purified by silica gel column chromatography (solvent: hexane/(ethyl acetate)=75/25-45/55), and thereby, the title compound (250 mg, 52%) as a white powder was obtained.

MS(ESI); m/z 1025.6 [M+H]+

### Reference Examples 14-29

The corresponding starting compounds were respectively treated in a similar manner as in Reference Example 2′ to obtain the compounds shown in Table 5 below.

TABLE 5

| Reference | | Physical property |
|---|---|---|
| Example | Structural formula | values and the like |
| 14 | 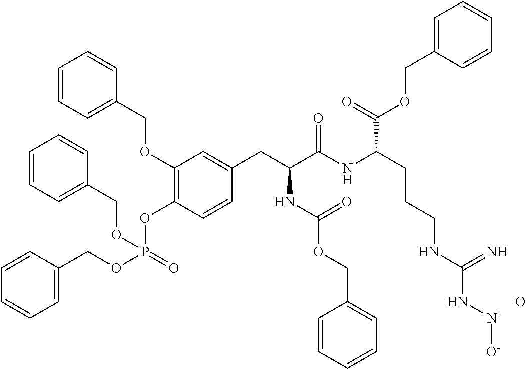 | MS (ESI); m/z 973.6 [M + H]+ |
| 15 | 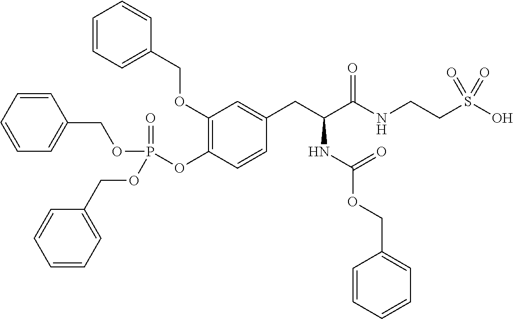 [SMILES: O=C(N[C@@H](Cc1ccc(OP(=O)(OCc2ccccc2)OCc2ccccc2)c(OCc2ccccc2)c1)C(=O)NCCS(=O)(=O)O)OCc1ccccc1] | MS (ESI); m/z 789.3 [M + H]+ |
| 16 | 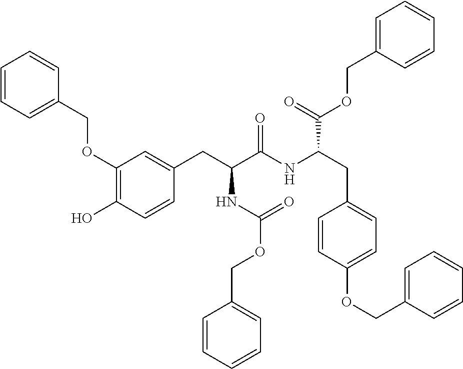 [SMILES: O=C(N[C@@H](Cc1ccc(O)c(OCc2ccccc2)c1)C(=O)N[C@@H](Cc1ccc(OCc2ccccc2)cc1)C(=O)OCc1ccccc1)OCc1ccccc1] | MS (ESI); m/z 765.7 [M + H]+ |
| 17 | 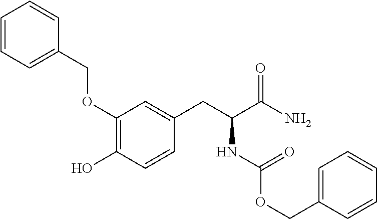 [SMILES: NC(=O)[C@H](Cc1ccc(O)c(OCc2ccccc2)c1)NC(=O)OCc1ccccc1] | MS (ESI); m/z 421.4 [M + H]+ |
| 18 | 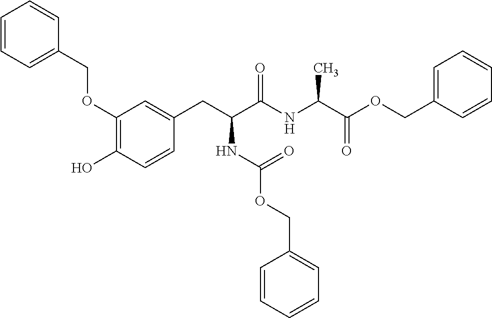 [SMILES: C[C@H](NC(=O)[C@H](Cc1ccc(O)c(OCc2ccccc2)c1)NC(=O)OCc1ccccc1)C(=O)OCc1ccccc1] | MS (ESI); m/z 583.5 [M + H]+ |
| 19 | 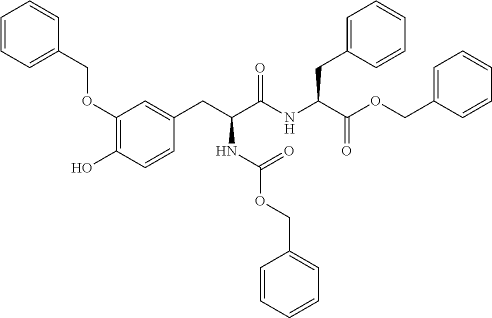 [SMILES: O=C(N[C@@H](Cc1ccc(O)c(OCc2ccccc2)c1)C(=O)N[C@@H](Cc1ccccc1)C(=O)OCc1ccccc1)OCc1ccccc1] | MS (ESI); m/z 659.5 [M + H]+ |
| 20 | 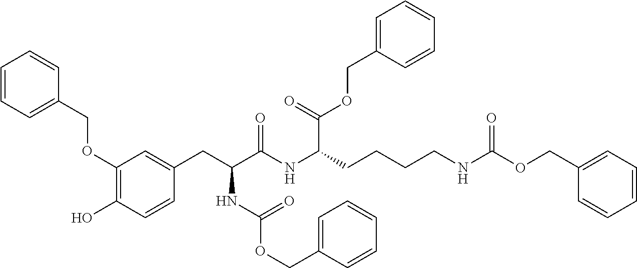 [SMILES: O=C(NCCCC[C@H](NC(=O)[C@H](Cc1ccc(O)c(OCc2ccccc2)c1)NC(=O)OCc1ccccc1)C(=O)OCc1ccccc1)OCc1ccccc1] | MS (ESI); m/z 774.4 [M + H]+ |
| 21 | 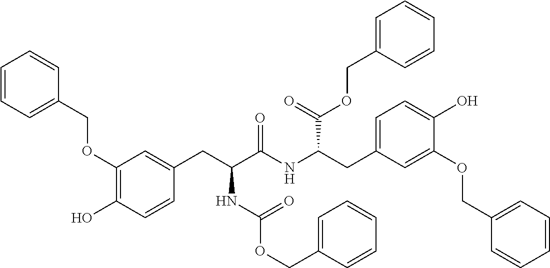 [SMILES: O=C(N[C@@H](Cc1ccc(O)c(OCc2ccccc2)c1)C(=O)N[C@@H](Cc1ccc(O)c(OCc2ccccc2)c1)C(=O)OCc1ccccc1)OCc1ccccc1] | MS (ESI); m/z 781.8 [M + H]+ |
| 22 | 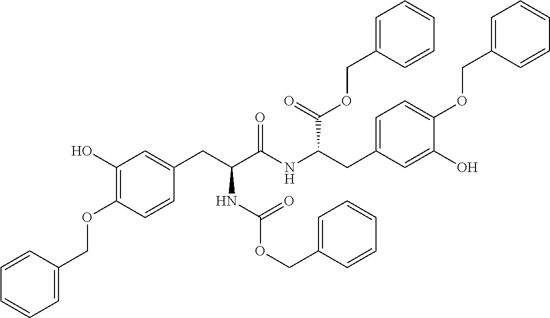 [SMILES: O=C(N[C@@H](Cc1ccc(OCc2ccccc2)c(O)c1)C(=O)N[C@@H](Cc1ccc(OCc2ccccc2)c(O)c1)C(=O)OCc1ccccc1)OCc1ccccc1] | MS (ESI); m/z 779.6 [M − H]− |
| 23 | 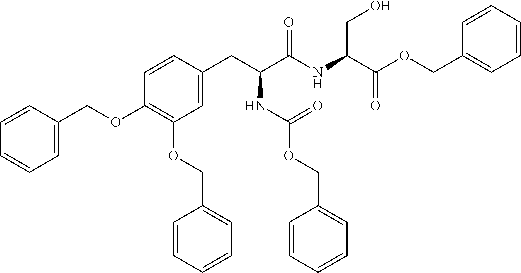 [SMILES: O=C(N[C@@H](Cc1ccc(OCc2ccccc2)c(OCc2ccccc2)c1)C(=O)N[C@@H](CO)C(=O)OCc1ccccc1)OCc1ccccc1] | MS (ESI); m/z 689.4 [M + H]+ |
| 24 | 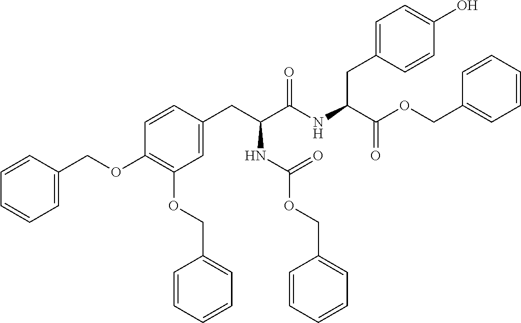 [SMILES: O=C(N[C@@H](Cc1ccc(OCc2ccccc2)c(OCc2ccccc2)c1)C(=O)N[C@@H](Cc1ccc(O)cc1)C(=O)OCc1ccccc1)OCc1ccccc1] | MS (ESI); m/z 765.5 [M + H]+ |
| 25 | 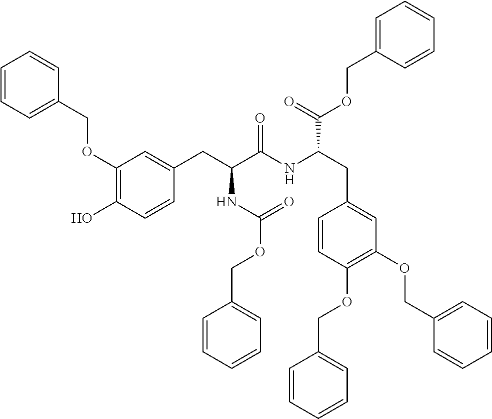 [SMILES: O=C(N[C@@H](Cc1ccc(O)c(OCc2ccccc2)c1)C(=O)N[C@@H](Cc1ccc(OCc2ccccc2)c(OCc2ccccc2)c1)C(=O)OCc1ccccc1)OCc1ccccc1] | MS (ESI); m/z 871.3 [M + H]+ |
| 26 | 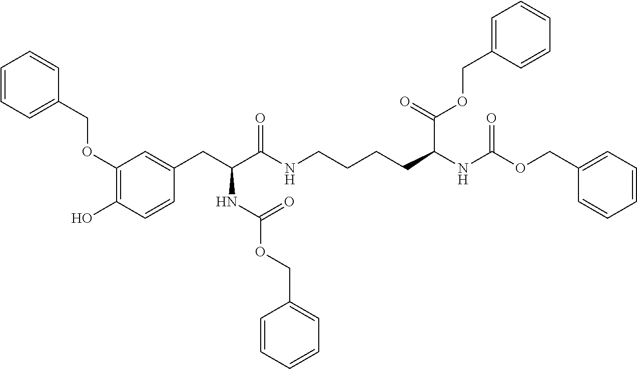 [SMILES: O=C(N[C@@H](Cc1ccc(O)c(OCc2ccccc2)c1)C(=O)NCCCC[C@H](NC(=O)OCc1ccccc1)C(=O)OCc1ccccc1)OCc1ccccc1] | MS (ESI); m/z 774.8 [M + H]+ |
| 27 | 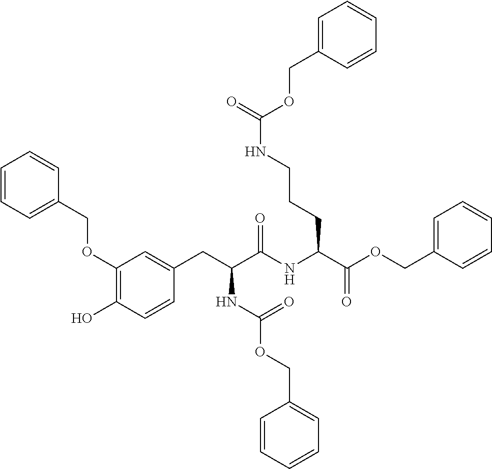 [SMILES: O=C(NCCC[C@H](NC(=O)[C@H](Cc1ccc(O)c(OCc2ccccc2)c1)NC(=O)OCc1ccccc1)C(=O)OCc1ccccc1)OCc1ccccc1] | MS (ESI); m/z 760.4 [M + H]+ |
| 28 | 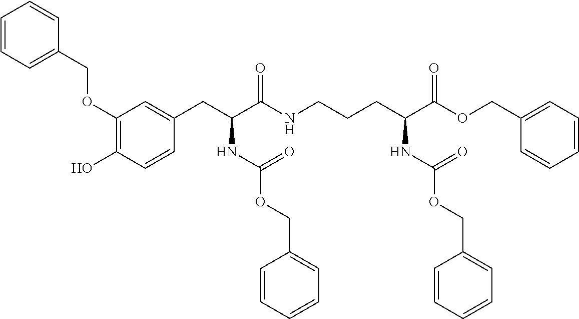 [SMILES: O=C(N[C@@H](Cc1ccc(O)c(OCc2ccccc2)c1)C(=O)NCCC[C@H](NC(=O)OCc1ccccc1)C(=O)OCc1ccccc1)OCc1ccccc1] | MS (ESI); m/z 760.4 [M + H]+ |
| 29 | 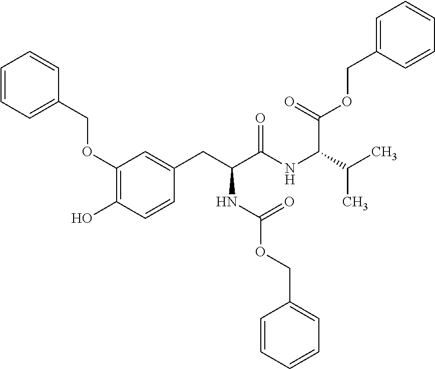 [SMILES: CC(C)[C@H](NC(=O)[C@H](Cc1ccc(O)c(OCc2ccccc2)c1)NC(=O)OCc1ccccc1)C(=O)OCc1ccccc1] | MS (ESI); m/z 611.6 [M + H]+ |

### Reference Example 30—Production of the Compound [a]

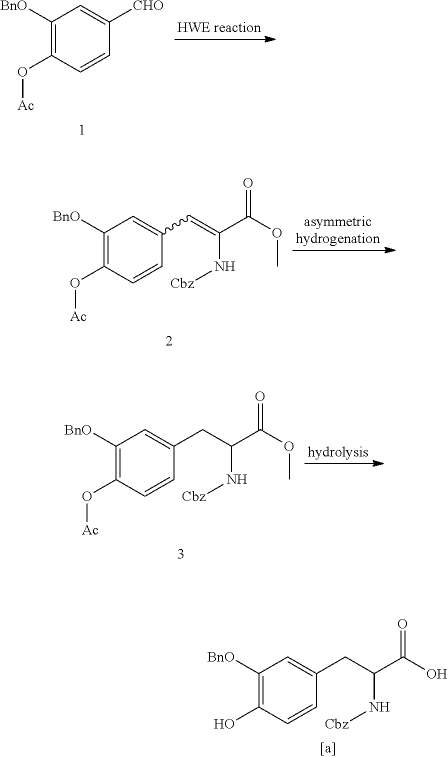 [SMILES: CC(=O)Oc1ccc(C=O)cc1OCc1ccccc1 ; COC(=O)C(=Cc1ccc(OC(C)=O)c(OCc2ccccc2)c1)NC(=O)OCc1ccccc1 ; COC(=O)C(Cc1ccc(OC(C)=O)c(OCc2ccccc2)c1)NC(=O)OCc1ccccc1 ; O=C(NC(Cc1ccc(O)c(OCc2ccccc2)c1)C(=O)O)OCc1ccccc1]

(1) The compound 1 (8.0 g, 74 wt %) was dissolved in dichloromethane (75 mL), and, under ice cooling, N-carbobenzoxy-2-phosphonoglycine trimethyl (7.98 g) and 1,1,3,3-tetramethylguanidine (3.6 mL) were added, and the mixture was stirred at room temperature for 16.5 hours. A saturated aqueous sodium hydrogen carbonate solution was added to the reaction mixture, and extraction with chloroform was performed. An organic layer was dried over sodium sulfate, and insoluble matter was filtered off, and then, the solvent was distilled away under a reduced pressure. The obtained residue was purified by silica gel column chromatography (solvent: hexane/(ethyl acetate)=85/15-65/35), and thereby, the compound 2 (8.58 g, 82%) as a white powder was obtained.

MS(ESI); m/z 476.4 [M+H]+

(2) The compound 2 (5.90 g, 92 wt %) was dissolved in tetrahydrofuran (80 mL), and (+)-1,2-bis((2S,5S)-2,5-diethylphosphorano)benzene(1,5-cyclooctadiene)rhodium(I) tetrafluoroborate ((S,S)-Et-DUPHOS-Rh) (295 mg) was added, and the mixture was stirred at room temperature under a pressurized hydrogen atmosphere (600 kPa) for 4 hours. The solvent of the reaction mixture was distilled away under a reduced pressure. The obtained residue was purified by silica gel column chromatography (solvent: hexane/(ethyl acetate)=85/15-55/45), and thereby, the compound 3 (5.71 g, 78%) as a white powder was obtained.

MS(ESI); m/z 478.4 [M+H]+

(3) The compound 3 (1.73 g) was dissolved in tetrahydrofuran (18 mL), methanol (9 mL) and distilled water (7 mL), and lithium hydroxide monohydrate (608 mg) was added, and the mixture was stirred at room temperature for 30 min. 1M Hydrochloric acid (30 mL) was added to the reaction mixture, and extraction with chloroform (50 mL) was performed. An organic layer was dried over sodium sulfate, and insoluble matter was filtered off, and then, the solvent was distilled away under a reduced pressure, and the compound [a] (1.69 g, 100%) as a white powder was obtained.

MS(ESI); m/z 422.4 [M+H]+

### Reference Examples 31—Production of the Compound [e]

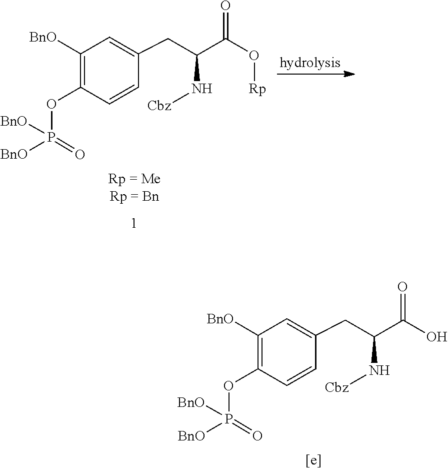

(1) The compound 1 (R=Me, 2.30 g) was dissolved in a mixed solvent of tetrahydrofuran (36 mL) and methanol (4 mL), and a 2M aqueous lithium hydroxide solution (4.0 mL) was added, and the mixture was stirred at room temperature for 10 min. The reaction mixture was ice-cooled, an 0.5 M aqueous potassium hydrogen sulfate solution (20 mL) was added, and extraction with chloroform was performed. An organic layer was washed with a saturated solution of sodium chloride and was dried over sodium sulfate, and insoluble matter was filtered off, and then, the solvent was distilled away under a reduced pressure. The residue was suspended in methyl tert-butyl ether, and a precipitated solid was collected by filtration and dried under a reduced pressure, and thereby, the compound [e] (2.30 g, 100%) as a white powder was obtained.

MS(ESI); m/z 682.6 [M+H]+

(1)′ The compound 1 (R=Bn, 0.57 g) was dissolved in methanol (2 mL), and an 1M aqueous sodium hydroxide solution (0.37 mL) was added, and the mixture was stirred at room temperature for 2 hours. The reaction mixture was acidified by adding a 1M hydrochloric acid, and then, extraction with ethyl acetate was performed. An organic layer was washed with water and a saturated solution of sodium chloride in this order, and dried over sodium sulfate, and insoluble matter was filtered off, and the solvent was distilled away under a reduced pressure, and thereby, the compound [e] (0.53 g, 104%) as a white powder was obtained.

MS(ESI); m/z 638.1 [M+H−CO2]+

### Reference Examples 32—Production of the Compound [b-1]

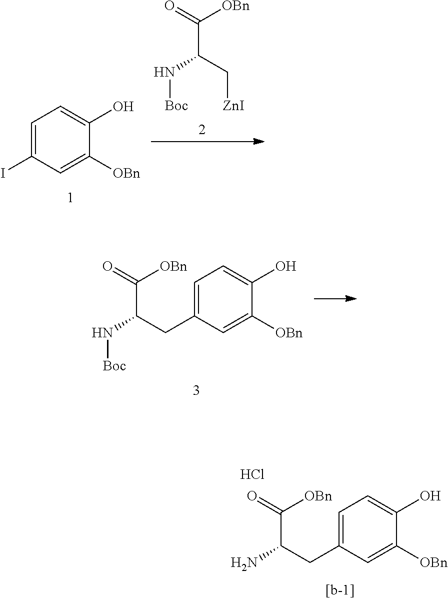

(1) Iodine (153 mg) was added to a suspension of activated zinc (923 mg) in N,N-dimethylformamide (7 mL) at 5° C. under a nitrogen atmosphere. The temperature was raised to 20° C. and the mixture was stirred for 10 minutes. The reaction mixture was cooled again to 6° C., and N-(tert-butoxycarbonyl)-3-iodo-L-alanine benzyl ester (1890 mg) was added in portions at 20° C. or below, and the mixture was stirred at 20° C. for 30 minutes, and thereby, a solution of the compound 2 was obtained.

Tris(dibenzylideneacetone)dipalladium(0)-chloroform adduct (31 mg), 2-dicyclohexylphosphino-2′,6′-dimethoxybiphenyldicyclohexyl(2′,6′-dimethoxy-[1,1′-biphenyl]-2-yl)phosphine (30 mg), and the compound 1 (1309 mg) were sequentially added, and the mixture was stirred at room temperature for 16 hours. Hexane/(ethyl acetate) (1:1) was added to the reaction mixture, and insoluble matter was removed by celite filtration. The insoluble matter was washed with hexane/(ethyl acetate) (1:1) and water, and the filtrate was washed sequentially with a saturated aqueous ammonium chloride solution and a saturated solution of sodium chloride. An organic layer was dried over anhydrous magnesium sulfate, and insoluble matter was filtered off, and then, the solvent was distilled away under a reduced pressure. The obtained residue was purified by silica gel column chromatography (solvent: hexane/(ethyl acetate)=80/20-67/33), and thereby, the compound 3 (1733 mg, 90%) as a white powder was obtained.

MS(ESI); m/z 378.2[M+H−Boc]+

(2) A 4M hydrogen chloride dioxane solution (6 mL) was added to a solution of the compound 3 (1.625 g) in 1,4-dioxane (15 mL) at 3° C., and the mixture was stirred at room temperature for 1 hour. A 4M hydrogen chloride dioxane solution (6 mL) was added, and the mixture was stirred for 17 hours. The reaction mixture was concentrated under a reduced pressure until the volume thereof was about 1/10. The residue was suspended in ethyl acetate, and a precipitated solid was collected by filtration and was dried under a reduced pressure, and thereby, the compound [b-1] (1271 mg, 90%) as a white powder was obtained.

MS(ESI); m/z 378.4[M+H]+

### Experimental Example 1—Solubility Studies

### Experimental Example 1.1—Solubility Studies of the LD-Tyr TFA Salt

LD-Tyr TFA salt, prepared according to Example 1, and which comprises one equivalent of TFA, was added to a solvent, as detailed in Table 6 below, and neutralized by addition of NaOH, where specified below. When maximum solubility was observed, i.e., additional LD-Tyr TFA salt added to the solution was not dissolved, the solution was filtered and then transferred to a bottle that was previously weighed and flushed with nitrogen. The volume of the solution in the bottle was adjusted to 5 ml, either by adding solvent, or be removing any residual solution, after which the bottle was tightly closed and left at 25° C. for stability observation. It is noted that throughout Example 4, the concentrations in the tables below were calculated taking into account the amount of solvent added or the solution removed, in which event, the calculations were performed regarding the 5 ml+(amount removed) as the volume of the solution. It is further noted that throughout this document, unless mentioned otherwise, the stability was measured using the manual visual inspection Bosch apparatus MIH DX, at a magnification of ×1.75.

TABLE 6
LD-Tyr TFA salt solubility results

| | LD-Tyr free | TFA counter- | LD-Tyr TFA | | | |
|---|---|---|---|---|---|---|
| | base | ion | salt | | | |
| | Concentration | Concentration | concentration | | | |
| Solvent^{A} | (mg/ml) | (mg/ml) | (mg/ml) | PH | Appearance | Stability |
| Water for | 160 | 50 | 210 | ~2 | clear | Stable |
| irrigation (WFI) | | | | | | overnight^{B} |
| 0.1% sodium | 160 | 45 | 205 | 7.22 | Precipitated | NA |
| bisulfite (Na Bis) | | | | | during | |
| in WFI | | | | | titration | |
| | | | | | (titer/NaOH) | |
| NMP | 150 | 50 | 200 | NA | Clear | NA |
| 1.2% CD | 135 | 43 | 178 | 8.35 | clear | Stable |
| solution^{C} in WFI | | | | (titer/NaOH) | | overnight |
| DMSO | 140 | 44 | 184 | NA | clear | NA |
| NMP: (0.1% | 150 | 50 | 200 | 4.62 | clear | Stable |
| NaBis in WFI) | | | | | | overnight |
| (70:30) | | | | | | |
| DMSO: (0.1% | 150 | 50 | 200 | 5.40 | clear | Stable |
| NaBis in WFI) | | | | | | overnight |
| (70:30) | | | | | | |

^{A}the solvent may include further excipients and APIs, such as CD, as listed in the tables, here and throughout
^{B}stable overnight = stable for a least 12 hours, here and throughout at room temperature
^{C}all CD solutions were prepared according to the procedure detailed in Example 5, here and throughout

As presented in Table 6, the solubility of the LD-Tyr TFA salt in water was 210 mg/ml; however, the pH was low. When raising the pH to about 7 with NaOH, the LD-Tyr TFA salt precipitated. The addition of cosolvents, such as NMP or DMSO allowed the pH to be elevated to physiologically acceptable values.

### Experimental Example 1.2—Solubility Studies of the LD-Tyr Free Base

The LD-Tyr free base, prepared according to Example 2, was added to a solvent, as detailed in Table 7 below, and neutralized by addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and left at 25° C. for stability observation.

TABLE 7
LD-Tyr free base solubility results

| | LD-Tyr free | | | |
|---|---|---|---|---|
| | base | | | |
| | Concentration | | | |
| Solvent | (mg/ml) | pH | Appearance | stability |
| WFI | <20 | <2 | clear | |
| Aqueous HCl | 75 | 6.85 | clear | Stable |
| (2 eq)* | | (titer/NaOH) | | overnight |
| Aqueous HCl | 150 | 2.2 | Precipitated | NA |
| (2 eq) * | | (titer/NaOH) | during | |
| | | | titration | |
| Mesylate | 75 | 6.85 | clear | Stable |
| | | (titer/NaOH) | | overnight |
| NMP | 300 | NA | clear | NA |
| DMSO | 220 | NA | clear | NA |
| DMSO:(0.75% | 125 | 5.22 | clear | Precipitated |
| CD solution) | | | | overnight** |
| (25:75) | | | | |
| NMP:(0.75% | 125 | 6.3 | clear | Precipitated |
| CD solution) | | | | overnight |
| (25:75) | | | | |

*the HCI was added to the water after the LD-Tyr free base was mixed into the water in order to dissolve the LD-Tyr free base, which did not dissolve when one HCI equivalent was used;
**precipitated overnight = precipitated within 12 hours at room temperature

As presented in Table 7, the solubility of the LD-Tyr free base in water was less than 20 mg/ml, and even that, at a low pH. However, elevating the pH to about 7 by the addition of NaOH, lead to precipitation. As further presented in Table 7, the addition of acids, such as hydrochloric acid or mesylate, provided higher solubility at physiological pH values; however, the solubility was still relatively limited in comparison to other molecules, as detailed herein. The use of other solvents, such as NMP and DMSO, provided higher solubility. In contrast, the addition of the CD solution lowered the solubility.

### Experimental Example 1.3—Solubility Studies of the LD-Tyr HCl Salt

LD-Tyr HCl salt, prepared according to Example 3, was added to a solvent, as detailed in Table 8 below, and neutralized by the addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and left at 25° C. for stability observation.

TABLE 8

| | LD-Tyr free | HCl counter- | LD-Tyr HCl | | | |
|---|---|---|---|---|---|---|
| | base | ion | salt | | | |
| | Concentration | Concentration | concentration | | | |
| Solvent | (mg/ml) | (mg/ml) | (mg/ml) | PH | Appearance | Stability |
| WFI | 260 | 40 | 300 | 8.46 | clear | Stable* |
| 0.75% CD | 257 | 40 | 297 | 8.65 | clear | Stable |
| solution | | | | | | overnight |

*Stable = stable for more than 12 hours at room temperature

As shown in Table 8, the stability of the LD-Tyr HCl salt is relatively high when compared to the free base and/or the TFA salt. In this respect it is noted that the addition of HCl to the free base, as presented in Experimental Example 1.2, would assumingly provide an LD-Tyr HCl salt in-situ and therefore, it would be expected that the solubility thereof would be at least similar to that of the LD-Tyr HCl solid salt, when dissolved, e.g., in water, or any other solvent. Nonetheless, when comparing the results presented in Tables 7 and 8 it is apparent that the solid LD-Tyr HCl salt (Table 8) is more soluble than the LD-Tyr HCl salt prepared in-situ (Table 7), and therefore, it is possible to dissolve a higher concentration of the solid LD-Tyr HCl salt at higher pH values.

Experimental Example 1.4—Solubility Studies of the LD-Arg TFA Salt

LD-Arg TFA salt, prepared according to Example 1, and which comprises two equivalents of TFA, was added to a solvent, as detailed in Table 9 below, and neutralized by addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and stored at 25° C. for stability observation.

TABLE 9

| | LD-Arg free | TFA counter- | LD-Arg TFA | | | |
|---|---|---|---|---|---|---|
| | base | ion | salt | | | |
| | Concentration | Concentration | concentration | | | |
| Solvent | (mg/ml) | (mg/ml) | (mg/ml) | PH | Appearance | Stability |
| WFI | 370 | 230 | 600 | 1.7 | clear | Stable |
| | | | | | | overnight |
| WFI | 260 | 160 | 420 | 7.2 | clear | Stable |
| | | | | | | overnight |
| 0.1% | 370 | 240 | 610 | 1.8 | clear | Stable |
| NaBis in | | | | | | overnight |
| WFI | | | | | | |
| 0.1% | 260 | 160 | 420 | 6.99 | clear | Stable |
| NaBis in | | | | | | overnight |
| WFI | | | | | | |
| WFI | 120 | 80 | 200 | 7.2 | clear | Stable |
| | | | | | | overnight |
| 0.1% | 120 | 80 | 200 | 7.06 | clear | Stable |
| NaBis in | | | | | | overnight |
| WFI | | | | | | |
| 0.1% | 120 | 80 | 200 | 6.92 | clear | Stable |
| NaBis in | | | | | | overnight |
| WFI | | | | | | |
| 1.2% CD | 110 | 70 | 180 | 8.48 | clear | Stable |
| solution | | | | | | overnight |

As presented in Table 9, the LD-Arg TFA salt demonstrated high solubility at low pH values; however, when pH was adjusted to physiological acceptable pH values, the solubility was reduced considerably.

### Experimental Example 1.5—Solubility Studies of the LD-Arg HCl Salt

LD-Arg HCl salt was added to a solvent, as detailed in Table 10 below, and neutralized by the addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and stored at 25° C. for stability observation.

TABLE 10

| | LD-Arg free | HCl counter- | LD-Arg HCl | | | |
|---|---|---|---|---|---|---|
| | base | ion | salt | | | |
| | Concentration | Concentration | concentration | | | |
| Solvent | (mg/ml) | (mg/ml) | (mg/ml) | PH | Appearance | Stability |
| WFI | 390 | 110 | 500 | 7.3 | clear | Stable |
| 0.2% NaBis | 440 | 110 | 550 | 7.02 | clear | Stable |
| in WFI | | | | | | overnight |
| 0.63% CD | 420 | 100 | 520 | 7.71 | clear | Stable |
| solution | | | | | | overnight |

As presented in Table 10, the solubility of the LD-Arg HCl salt is relatively high, when comparing to other free bases and/or salts tested herein, even at physiologically acceptable pH values.

### Experimental Example 1.6—Solubility Studies of the LD-Lys TFA Salt

LD-Lys TFA salt, prepared according to Example 1, and which comprises two equivalents of TFA, was added to a solvent, as detailed in Table 11 below, and neutralized by addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and stored at 25° C. for stability observation.

TABLE 11

| | LD-Lys free | TFA counter- | LD-Lys TFA | | | |
|---|---|---|---|---|---|---|
| | base | ion | salt | | | |
| | Concentration | Concentration | concentration | | | |
| Solvent | (mg/ml) | (mg/ml) | (mg/ml) | pH | Appearance | Stability |
| WFI | 450 | 320 | 770 | 1.86 | clear | Stable |
| | | | | | | overnight |
| WFI | 350 | 250 | 600 | 7.1 | clear | Stable |
| | | | | | | overnight |
| 0.1% | 310 | 210 | 520 | 7.0 | clear | Stable |
| NaBis | | | | | | overnight |
| in WFI | | | | | | |
| 0.75% | 150 | 100 | 250 | 7.4 | precipitated | Precipitated* |
| CD | | | | | | |
| solution | | | | | | |
| 0.75% | 150 | 100 | 250 | 6.5 | precipitated | Precipitated* |
| CD | | | | | | |
| solution | | | | | | |
| 0.75% | 120 | 85 | 205 | 7.4 | clear | Stable |
| CD | | | | | | overnight |
| solution | | | | | | |
| 1.2% | 110 | 70 | 180 | 8.36 | clear | Stable |
| CD | | | | | | overnight |
| solution | | | | | | |

*precipitated within 12 hours

As presented in Table 11, the solubility of the LD-Lys TFA salt is relatively high, when comparing to other free bases and/or salts tested herein; however, when elevating the pH to physiologically acceptable pH values, the solubility of the LD-Lys TFA salt is reduced.

### Experimental Example 1.7—Solubility Studies of the LD-Lys Free Base

The LD-Tyr free base, prepared according to Example 2, was added to a solvent, as detailed in Table 12 below; however, visual assessment of the solution showed that the LD-Tyr did not dissolve. Heating to 70° C. was employed in order to improve solubility; however, this was insufficient, since, even after heating, precipitants were viewed. As presented in Table 12 below, of the solutions that were tested, only when two equivalents of TFA were added, the addition of NaOH up to a pH of 6.8 provided a solution that was stable overnight, i.e., for at least 12 hours. As detailed above regarding other solutions, with the LD-Tyr free base when maximum solubility was observed, the solution was filtered and then transferred to a bottle that was previously weighed and flushed with nitrogen. The volume of the solution in the bottle was adjusted to 5 ml, after which the bottle was tightly closed and left at 25° C. for stability observation.

TABLE 12

| | LD-Lys free base | | | |
|---|---|---|---|---|
| | Concentration | | | |
| Solvent | (mg/ml) | pH | Appearance | Stability |
| WFI (heated | 50 | | Precipitated | — |
| to 70° C.) | | | | |
| TFA(2 eq) | 50 | 6.8 | clear | Stable |
| | | | | overnight |
| TFA(2 eq) | 150 | 6.8 | clear | Stable |
| | | | | overnight |
| TFA(2 eq) | 350 | 1.17 | Precipitated | — |
| (heated to | | | | |
| 60° C.) | | | | |
| Buffer (heated | 5 | | Precipitated | — |
| to 70° C.), pH | | | | |
| 8.8 | | | | |
| HC1 (2 eq) | 75, 150 | | Precipitated | — |
| (heated to | | | | |
| 700° C.) | | | | |
| Mesylate (2 eq) | 75, 150 | | Precipitated | — |

As presented in Table 12, the LD-Lys free base demonstrated very low solubility which was unexpected. It is noted that even when acids were added, supposedly forming an in-situ salt, e.g., an HCl or TFA salt, the solubility remained low. When comparing this to the results presented in Tables 11 and 13 herein, it appears that the solubility of the LD-Lys salts, prepared in solid form, is substantially different than those salts, when prepared in-situ by the addition of an acid to the free base solution. For example, while, as presented in Table 11, the WFI solution of 600 mg/ml of LD-Lys TFA salt, comprising 350 mg/ml LD-Lys free base and two TFA equivalents, is stable for at least 12 hours, it was not possible to dissolve the same amount of the LD-Lys free base to which two equivalents of TFA were added even with the aid of heating (see Table 12). This is similar to the findings detailed above regarding the in-situ preparation of the LD-Tyr salts, evident from comparing the results presented in Tables 7 and 8. The significant differences between the solubility results obtained for the solid LDAA salts and the in-situ LDAA salts is highly unexpected.

### Experimental Example 1.8—Solubility Studies of the LD-Lys HCl Salt

LD-Lys HCl salt, prepared according to Example 3, was added to a solvent, as detailed in Table 13 below, and neutralized by the addition of NaOH. When maximum solubility was observed, the solution was filtered and then transferred to a bottle that was previously weighed and flushed with nitrogen. The volume of the solution in the bottle was adjusted to 5 ml, after which the bottle was tightly closed and left at 25° C. for stability observation.

TABLE 13

| | LD-Lys free | HCl counter- | LD-Lys TFA | | | |
|---|---|---|---|---|---|---|
| | base | ion | salt | | | |
| | Concentration | Concentration | concentration | | | |
| Solvent | (mg/ml) | (mg/ml) | (mg/ml) | pH | Appearance | Stability |
| WFI | 290 | 60 | 350 | 6.51 | Clear | Stable |
| 0.75% | 200 | 40 | 240 | 6.46 | Clear | Stable |
| CD | | | | | | overnight |
| solution | | | | | | |
| 0.75% | 200 | 40 | 240 | 8.5 | Clear | Precipitated* |
| CD | | | | | | |
| solution | | | | | | |

*precipitated within 12 hours at room temperature

As presented in Table 13, the LD-Lys HCl salt demonstrated high solubility even at pH values above 6; however, when pH raised to 8.5, the solubility of the LD-Lys HCl salt was reduced.

### Experimental Example 2—LD-Arg, LD-Lys and LD-Tyr Formulations

### Experimental Example 2.1—LD-Tyr and Carbidopa (CD) Formulations

### Carbidopa Solution Preparation

First, a carbidopa (CD) solution was prepared by mixing sodium bisulfite, WFI and Tween® 80. The obtained clear solution was heated to 60° C. Carbidopa was added, the flask nitrogen flushed and stirred to allow the homogenous dispersion of the CD. NaOH was added until required pH was obtained, the flask was nitrogen flushed again, tightly closed, and the mixture therein stirred for ten minutes at 60° C. The flask was allowed to cool to room temperature. The pH of the obtained solution was measured and adjusted if necessary. The solution was then transferred to a bottle for weighing, volume completion, and final weight determination, after which the solution was transferred to vials that were purged with nitrogen at head space, tightly closed and stored at −20° C.

### CD/LD-Tyr HCl Salt Solution Preparation

CD/NaOH solution was transferred into a vial and stirred. LD-Tyr HCl salt, prepared according to Example 3, was added in portions (approximately 100-150 mg) to the vial while stirring, with constant pH monitoring. Once the added portion of LD-Tyr HCl salt dissolved, the pH was adjusted to 8.4±0.1 by adding NaOH to the solution. When all of the LD-Tyr HCl salt was dissolved, the solution was transferred to a bottle, the volume was adjusted to the size of the bottle (e.g., 5 ml, 10 ml, 20 ml) by the addition of WFI, the final weight and volume were recorded, the solution was filtered, the head space was purged with nitrogen, tightly closed, and stored at 25° C.

### CD/LD-Tyr Free Base Solution Preparation

A dispersion of Tween® 80 in NMP was prepared by adding Tween® 80 to NMP and stiffing. The LD-Tyr free base, prepared according to Example 2, was added in portions, until maximum dissolution was reached (the solution may appear cloudy at maximum dissolution). When all of LD-Tyr free base was added, the CD solution, prepared as detailed above, was added, and volume was completed with WFI. The pH was measured, the solution was transferred to a bottle, the volume was adjusted to the size of the bottle by the addition of WFI, the final weight was recorded, the solution was filtered, and the head space was purged with nitrogen, tightly closed, and stored at 25° C.

TABLE 14

| | CD/LD-Tyr | CD/LD-Tyr HCl |
|---|---|---|
| Composition % | free base (F1) | salt (F2) |
| LD-Tyr free base | 12.5 | — |
| HCl counter ion | — | — |
| LD-Tyr HCl salt | | 17.4 (comprising the |
| | | equivalent of 15% |
| | | LD-Tyr free base and |
| | | 2.4% HCl counter ion) |
| Carbidopa (on a dry | 0.75 | 0.75 |
| basis) | | |
| Sodium bisulfite | 0.13 | 0.15 |
| Tween ® 80 | 0.30 | 0.30 |
| N-Methylpyrrolidone | 25 | 0 |
| Sodium hydroxide | 0.20 | 4.11 |
| WFI | QS to 100 | QS to 100 |
| Final pH | 6.6 | 8.5 |
| Stability | Stable | Stable overnight |
| | overnight | |

### Example 2.2—LD-Arg and Carbidopa (CD) Formulations

### CD/LD-Arg HCl Salt Solution Preparation

A CD solution was prepared according to the procedure described in Example 5.1. The CD solution was transferred into a vial and stirred. LD-Arg HCl salt was added in two portions, with constant pH monitoring. Once the added portions of LD-Arg HCl salt dissolved, the pH was adjusted to 7.1±0.2 by adding NaOH to the solution. When all of the LD-Arg HCl salt was dissolved, the solution was transferred to a bottle, the volume was adjusted to the size of the bottle by adding WFI, the final weight was recorded, the solution was filtered, and the head space was purged with nitrogen, tightly closed, and stored at 25° C.

TABLE 15

| | CD/LD-Arg | CD/LD-Arg | CD/LD-Arg |
|---|---|---|---|
| Composition % | HCl salt (F3) | HCl salt (F4) | HCl salt (F5) |
| LD-Arg HCl salt | 14.60 | 26.80 | 36.50 |
| | (comprising the | (comprising the | (comprising the |
| | equivalent of | equivalent of | equivalent of |
| | 12% LD-Arg | 22% LD-Arg | 30% LD-Arg |
| | free base and | free base and | free base and |
| | 2.6% HCl | 4.8% HCl | 6.5% HCl |
| | counter ion) | counter ion) | counter ion) |
| Carbidopa (on dry | 0.75 | 0.75 | 0.75 |
| basis) | | | |
| Na Bisulfite | 0.15 | 0.15 | 0.15 |
| Sodium hydroxide | 1.2 | 2 | 2.8 |
| Tween ® 80 | 0.30 | 0.30 | 0.30 |
| WFI | QS to 100 | QS to 100 | QS to 100 |
| Final pH | 7.02 | 7.05 | 7.22 |
| Stability | Stable | Stable | Stable |
| | overnight | overnight | overnight |

### Experimental Example 2.3—LD-Lys and Carbidopa (CD) Formulations

### CD/LD-Lys HCl Salt Solution Preparation

A CD/NaOH solution was prepared according to the procedure described in Experimental Example 2.1. CD solution was transferred into a vial and stirred. LD-Lys HCl salt, prepared according to Example 3, was added portion wise, with constant pH monitoring. Once the added portions of LD-Lys HCl salt dissolved, the pH was adjusted to 6.7±0.2 by adding NaOH to the solution. When all of the LD-Lys HCl salt was dissolved, the solution was transferred to a bottle, the volume was adjusted to the size of the bottle by the addition of WFI, the solution was filtered, the head space was purged with nitrogen, the bottle was tightly closed, and stored at 25° C.

TABLE 16

| | CD/LD-Lys | CD/LD-Lys | CD/LD-Lys | CD/LD-Lys | CD/LD-Lys |
|---|---|---|---|---|---|
| Composition | HCl salt | HCl salt | HCl salt | HCl salt | HCl salt |
| % | (F6) | (F7) | (F8) | (F9) | (F10) |
| LD-Lys HCl | 18.2 | 18.2 | 24.3 | 18.2 | 12.1 |
| salt | (comprising the | (comprising the | (comprising the | (comprising the | (comprising the |
| | equivalent of | equivalent of | equivalent of | equivalent of | equivalent of |
| | 15% LD-Lys | 15% LD-Lys | 20% LD-Lys | 15% LD-Lys | 10% LD-Lys |
| | free base and | free base and | free base and | free base and | free base and |
| | 3.2% HCl | 3.2% HCl | 4.3% HCl | 3.2% HCl | 2.14% HCl |
| | counter ion) | counter ion) | counter ion) | counter ion) | counter ion) |
| Carbidopa | 0 | 0.75 | 0.75 | 0.75 | 0 |
| (dry) | | | | | |
| Na Bisulfite | 0.15 | 0.15 | 0.15 | 0.15 | 0 |
| Sodium | 2.5 | 2.35 | 3.1 | 3.2 | 2.72 |
| hydroxide | | | | | |
| Tween ® 80 | 0.30 | 0.30 | 0.3 | 0.3 | 0 |
| WFI | QS to 100 | QS to 100 | QS to 100 | QS to 100 | QS to 100 |
| Final pH | 6.57 | 6.69 | 6.46 | 7.41 | 8.15 |
| Stability | Stable | Stable | Stable | Precipitated | Precipitated |
| | overnight | overnight | overnight | | |

### Experimental Example 3

### In-Vitro Metabolism of LDAA Compounds Using Liver Microsomes

The stability of test compounds in pooled human liver microsomes was determined on 96-well plates, wherein the test compounds were quantified at five time points by HPLC-MS/MS analysis. The assay matrix included mixed gender and a pool of 50 human liver microsomes, wherein the final microsomal protein concentration was 0.1 mg/mL. The test concentration was 0.1 μM with 0.01% DMSO, 0.25% acetonitrile and 0.25% methanol.

Each test compound was pre-incubated for five minutes with pooled liver microsomes in phosphate buffer (pH 7.4) in a 37° C. shaking water-bath. The reaction was initiated by adding a nicotinamide adenine dinucleotide phosphate (NADPH)-generating system and incubating for 0, 15, 30, 45, and 60 min. The reaction was stopped by transferring the incubation mixture to acetonitrile/methanol. Samples were then mixed and centrifuged, wherein the supernatants were used for HPLC-MS/MS analysis.

In each assay the four reference compounds propranolol, imipramine, verapamil and terfenadine were tested, wherein the propranolol and the imipramine are known to be relatively stable, while the verapamil and the terfenadine are known to be readily metabolized in human liver microsomes.

All samples were analyzed by HPLC-MS/MS using selected reaction monitoring. The HPLC system consisted of a binary LC pump with autosampler, a C-18 column, and a gradient. The conditions were adjusted when necessary.

Peak areas corresponding to the test compound were recorded. The amount of each compound remaining was calculated by comparing the peak area at each time point to time zero. The half-life is calculated from the slope of the initial linear range of the logarithmic curve of compound remaining (%) vs. time, assuming first order kinetics. In addition, the intrinsic clearance (Clᵢₙₜ) was calculated from the half-life using the following equation:

Clᵢₙₜ(μL/min/mg protein)=0.693/(t_{1/2}×protein concentration)

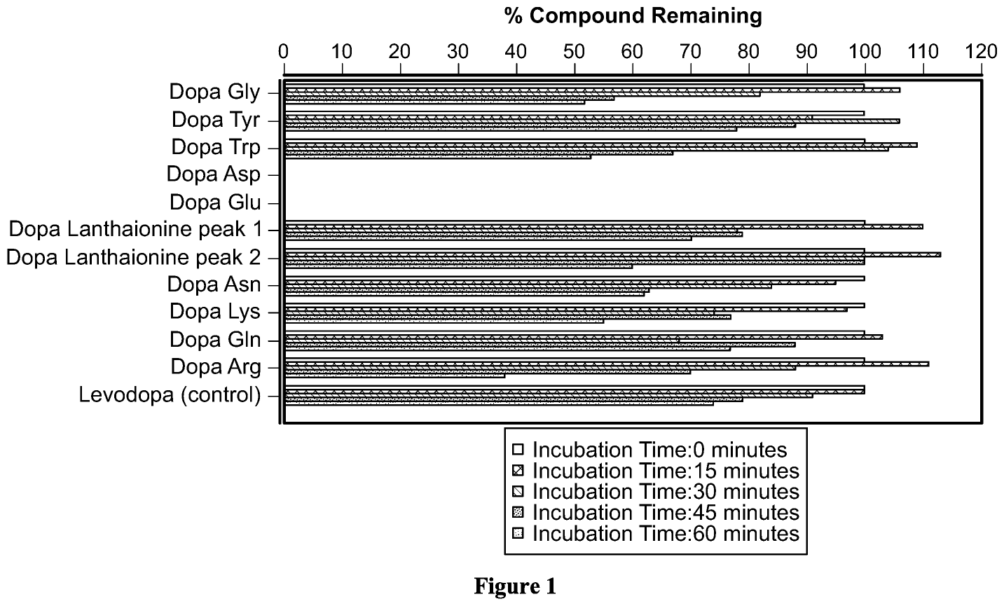

The results of the in-vitro human liver microsome metabolism tests of various LDAA compounds (10⁻⁷M), in their TFA salt forms, are provided in FIG. 1 and in Table 17, which presents the % of the compound remaining at times 0, 15, 30, 45 and 60 minutes, two half-life measurements and the Clᵢₙₜ, calculated as detailed above. It is noted that while FIG. 1 does not explicitly mention the TFA salt forms, the results presented therein are relevant to the TFA salts, i.e., Dopa Gly is what is referred to herein as LD-Gly TFA salt, etc.

TABLE 17

| | | Incubation | % Compound | | | | | | | |
|---|---|---|---|---|---|---|---|---|---|---|
| | Test | Time | Remaining | | | Half-Life (minute) | | | | |
| Compound | Concentration | (minutes) | 1^{st} | 2^{nd} | Mean | 1^{st} | 2^{nd} | Mean | Clint | Flags |
| LD-Gly TFA salt | 1.0E−07M | 0 | 100 | 100 | 100 | 54.5 | 54.1 | 54 | 127.7 | |
| | | 15 | 103.7 | 108.1 | 106 | | | | | |
| | | 30 | 86.7 | 77.9 | 82 | | | | | |
| | | 45 | 57.5 | 56.4 | 57 | | | | | |
| | | 60 | 51.7 | 52.9 | 52 | | | | | |
| LD-Tyr TFA salt | 1.0E−07M | 0 | 100 | 100 | 100 | 111.6 | 314.9 | >60 | <115.5 | |
| | | 15 | 90.7 | {121.6 | 91 | | | | | |
| | | 30 | 102.5 | 110.1 | 106 | | | | | |
| | | 45 | 71.4 | 104.1 | 88 | | | | | |
| | | 60 | 70.8 | 84.5 | 78 | | | | | |
| LD-Trp TFA salt | 1.0E−07M | 0 | 100 | 100 | 100 | 64 | 61.4 | >60 | <115.5 | |
| | | 15 | 108.6 | {96.6} | 109 | | | | | |
| | | 30 | 115.2 | 92.2 | 104 | | | | | |
| | | 45 | 70.5 | 63.1 | 67 | | | | | |
| | | 60 | 55.1 | 51.6 | 53 | | | | | |
| LD-Asp TFA salt | 1.0E−07M | 0 | | | | | | | | ND |
| | | 15 | | | | | | | | ND |
| | | 30 | | | | | | | | ND |
| | | 45 | | | | | | | | ND |
| | | 60 | | | | | | | | ND |
| LD-Glu TFA salt | 1.0E−07M | 0 | | | | | | | | ND |
| | | 15 | | | | | | | | ND |
| | | 30 | | | | | | | | ND |
| | | 45 | | | | | | | | ND |
| | | 60 | | | | | | | | ND |
| LD-LA1 TFA salt | 1.0E−07M | 0 | 100 | 100 | 100 | 138.9 | 87.3 | >60 | <115.5 | |
| | | 15 | {129.4 | 110.4 | 110 | | | | | |
| | | 30 | {125.6 | 77.6 | 78 | | | | | |
| | | 45 | 79.7 | 77.9 | 79 | | | | | |
| | | 60 | 74.2 | 65.6 | 70 | | | | | |
| LD-LA2 TFA salt | 1.0E−07M | 0 | 100 | 100 | 100 | 75.7 | 110.3 | >60 | <115.5 | |
| | | 15 | 122.9 | 103.5 | 113 | | | | | |
| | | 30 | 105.6 | 94.5 | 100 | | | | | |
| | | 45 | 97.5 | 102 | 100 | | | | | |
| | | 60 | 56.5 | 62.9 | 60 | | | | | |
| LD-Asn TFA salt | 1.0E−07M | 0 | 100 | 100 | 100 | 74.4 | 77.3 | >60 | <115.5 | |
| | | 15 | 92.8 | 96.4 | 95 | | | | | |
| | | 30 | 79.9 | 88.6 | 84 | | | | | |
| | | 45 | 66.1 | 59.7 | 63 | | | | | |
| | | 60 | 58.9 | 64.9 | 62 | | | | | |
| LD-Lys TFA salt | 1.0E−07M | 0 | 100 | 100 | 100 | 91.3 | 58.3 | >60 | <115.5 | |
| | | 15 | {123.5 | 96.7 | 97 | | | | | |
| | | 30 | 64.2 | 83.5 | 74 | | | | | |
| | | 45 | 76.7 | {43.7} | 77 | | | | | |
| | | 60 | 59.5 | 50.4 | 55 | | | | | |
| LD-Gln TFA salt | 1.0E−07M | 0 | 100 | 100 | 100 | 155 | 158.1 | >60 | <115.5 | |
| | | 15 | 103.4 | {121.2 | 103 | | | | | |
| | | 30 | 68.2 | {139.0 | 68 | | | | | |
| | | 45 | 81.8 | 94.1 | 88 | | | | | |
| | | 60 | 80.4 | 72.8 | 77 | | | | | |
| LD-Arg TFA salt | 1.0E−07M | 0 | 100 | 100 | 100 | 42.9 | 40.5 | 42 | 166.3 | |
| | | 15 | 109.7 | 111.8 | 111 | | | | | |
| | | 30 | 84.2 | 90.9 | 88 | | | | | |
| | | 45 | 70 | {52.4} | 70 | | | | | |
| | | 60 | 37.3 | 38.6 | 38 | | | | | |
| LD (control) | 1.0E−07M | 0 | 100 | 100 | 100 | 116.2 | 130.6 | >60 | <115.5 | |
| | | 15 | 102.5 | 97.8 | 100 | | | | | |
| | | 30 | 93.2 | 89.3 | 91 | | | | | |
| | | 45 | 80.1 | 78 | 79 | | | | | |
| | | 60 | 72.3 | 75.2 | 74 | | | | | |

As presented in Table 17 and in FIG. 1, the LD-Arg TFA salt provided the highest intrinsic clearance (Clᵢₙₜ), i.e., 166.3 μL/min/mg protein. The LD-Gly TFA salt provided a Clᵢₙₜ of 127.7 uL/min/mg. as further presented, both the LD-Gln and the LD-Asp TFA salts were not detected. The remaining tested LDAA compounds provided a Clᵢₙₜ value lower than 115.5 μL/min/mg protein.

### Experimental Example 4

### Human Liver S9 Stability Test

The stability of several LDAA compounds, in their TFA salt form, in human liver S9 was tested using commercially available liver S9. The substrate concentration was 10 μM, the S9 protein concentration was 0.2 mg/mL, and the incubation time 0, 5, 15, 30 and 60 minutes.

The results are described in Table 18, wherein the results describe the Ke, i.e., the slope of the percentage decrease of the remaining amount of the compound, measured at each of the above time points, such that the higher the Ke the faster the metabolism.

TABLE 18

| Compound | Ke |
|---|---|
| LDA (levodopa amide) | −0.0008 |
| LD-Arg TFA salt | 0.1268 |
| LD-Lys TFA salt | 0.0788 |
| LD-Asn TFA salt | 0.0015 |
| LD-Asp TFA salt | 0.0008 |
| LD-Tyr TFA salt | 0.0217 |

As shown in Table 18, the TFA salts of LD-Arg, LD-Lys and LD-Tyr were rapidly metabolized in human liver S9.

### Experimental Example 5

### Human Blood Stability Test

The stability of several LDAA compounds in human blood was tested. The substrate concentration was 10 μM and the incubation time 0, 5, 15, 30 and 60 minutes.

The results are described in Table 19, wherein the results describe the Ke, i.e., the slope of the percentage decrease of the remaining amount of the compound, measured at each of the above time points, such that the higher the Ke the faster the metabolism.

TABLE 19

| Compound | Ke |
|---|---|
| LDA (levodopa amide) | 0.0149 |
| LD-Arg TFA salt | 0.0919 |
| LD-Lys TFA salt | 0.0655 |
| LD-Asn TFA salt | 0.0038 |
| LD-Asp TFA salt | 0.0014 |
| LD-Tyr TFA salt | 0.0426 |
| LD-LA 1 TFA salt | −0.011 |
| LD-LA 2 TFA salt | 0.0128 |

As shown in Table 19, all compounds, except for LD-Asp, LD-LA 1, and less so, LD-Asn, were rapidly metabolized.

### Experimental Example 6

### Protein Binding—Equilibrium Dialysis Method

The protein binding values of various LDAA compounds in their TFA salt form (10⁻⁵M) were tested in human plasma. The equilibrium dialysis technique was used to separate the fraction of the test compound that was unbound from the fraction of the test compound that bound to proteins during the test. The test was performed on 96-well plates in a dialysis block constructed from Teflon™.

The protein containing matrix used was human plasma, wherein the assay matrix was human serum albumin and alpha-1 acid glycoprotein. The protein matrix was spiked with each test compound at 10 μM (by default, n=2) with a final DMSO concentration of 1%. The dialysate compartment is loaded with phosphate buffered saline (PBS, pH 7.4), and the sample compartment was loaded with an equal volume of the spiked protein matrix. The dialysis plate was then sealed and incubated at 37° C. for 4 h.

Following the incubation, samples were taken from each compartment, diluted with PBS followed by the addition of acetonitrile, after which the samples were centrifuged. The supernatants were collected and analyzed by HPLC-MS/MS. The HPLC tests included a binary LC pump with an autosampler, a C18 column (2×20 mm), and gradient elution. The HPLC conditions were adjusted when necessary.

A control sample (n=2) was prepared from the spiked protein matrix in the same manner; however, no dialysis was performed on the control. It is noted that the control sample served as the bases for the recovery determination.

Acebutolol, quinidine, and warfarin were used in each assay as reference compounds, wherein it is known that those reference compounds provide low, medium and high human plasma protein binding values, respectively.

The % of the tested compound that is bound to proteins and the recovery values were calculated as follows:

Protein binding (%)=100×(Areaₚ−Area_{b})/Areaₚ

Recovery (%)=100×(Areaₚ−Area_{b})/Area_{c}

Areaₚ=peak area of analyte in the protein matrix;

Area_{b}=peak area of analyte in the assay buffer; and

Area_{c}=peak area of analyte in the control sample.

The determination of the recovery % serves as an indicator of reliability of the calculated protein binding value. Low recovery indicates that the test compound is lost during the course of the assay. This is most likely due to non-specific binding or degradation of the test compound. It is noted that a recovery of above 60% is considered to be reliable, while under 60% recovery, the results of the test are considered to be unreliable.

The results of the protein binding tests are presented in Table 20 below.

TABLE 20

| | Test | % Protein Bound | | | % Recovery | | |
|---|---|---|---|---|---|---|---|
| Compound | Concentration | 1^{st} | 2^{nd} | Mean | 1^{st} | 2^{nd} | Mean |
| LD-Gly TFA salt | 1.0E-05M | ND | ND | ND | ND | ND | ND |
| LD-Tyr TFA salt | | ND | ND | ND | ND | ND | ND |
| LD-Trp TFA salt | | 54.8 | 66 | 60 | 68 | 74 | 71 |
| LD-Asp TFA salt | | 99.1 | 99.4 | 99 | 105 | 108 | 107 |
| LD-Glu TFA salt | | ND | ND | ND | ND | ND | ND |
| LD-LA1 TFA salt | | 24.4 | 27.8 | 26 | 75 | 71 | 73 |
| LD-LA2 TFA salt | | ND | ND | ND | ND | ND | ND |
| LD-Asn TFA salt | | ND | ND | ND | ND | ND | ND |
| LD-Lys TFA salt | | 59.5 | 53.1 | 56 | 12 | 19 | 16 |
| LD-Gln TFA salt | | 99.7 | 99.8 | 99 | 77 | 78 | 78 |
| LD-Arg TFA salt | | 44.1 | 26.4 | 35 | 6 | 5 | 6 |
| LD (control) | | ND | ND | ND | ND | ND | ND |

As mentioned above, when the % recovery is below 60% or above 100%, the test results are considered to be unreliable and therefore, the results presented in Table 20 regarding the LD-Lys TFA salt and the LD-Arg TFA salt are considered to be unreliable. In view of the low reliability of certain results, and in view of the fact that some compounds were not detected by the above tests, a second method (SPE method) for measuring protein binding was performed.

### Experimental Example 7

### Protein Binding—Solid Phase Extraction (SPE) Method

A solid phase extraction (SPE) method was used to prepare samples for several compounds in the plasma protein binding assay. The following SPE protocols were followed:

### SPE Protocol 1—Mixed Mode, Cation Exchange (Performed for LD-Lys TFA Salt and LD-Arg Tfa Salt)

Sorbent: Waters Oasis MCX 96—well MicroElution Plate—Cat #186001830BA
Sample: 200 μL of plasma spiked at 10 μM with test compound. Sample was diluted 1:1 with 4% phosphoric acid in water and mixed for 15 minutes
1) Place Oasis plate on vacuum manifold and set vacuum to 5″ Hg;
2) Condition with 200 μL methanol;
3) Equilibrate with 200 μL water;
4) Load dilute plasma sample;
5) Wash with 200 μL 2% formic acid in water;
6) Wash with 400 μL methanol; and
7) Elute with 100 μL 5% NH₄OH in methanol.

### SPE Protocol 2—Mixed Mode, Anion Exchange (Performed for LD-Tyr TFA Salt, LD-Asp TFA Salt, LD-Glu TFA Salt, LD-LA 2 TFA Salt and Levodopa)

Sorbent: Waters Oasis MAX 96-well MicroElution plate—Cat #186001829
Sample: 200 μL of plasma spiked at 10 μM with test compound. Sample was diluted 1:1 with 4% phosphoric acid in water and mixed for 15 minutes
1) Place Oasis plate on vacuum manifold and set vacuum to 5″ Hg;
2) Condition with 200 μL methanol;
3) Equilibrate with 200 μL water;
4) Load dilute plasma sample;
5) Wash with 200 μL 5% NH₄OH in water;
6) Wash with 400 μL methanol; and
7) Elute with 100 μL 2% formic acid in methanol.

The results of the protein binding SPE tests are presented in Table 21 below.

TABLE 21

| | Test | % Protein Bound | | | % Recovery | | |
|---|---|---|---|---|---|---|---|
| Compound | Concentration | 1^{st} | 2^{nd} | Mean | 1^{st} | 2^{nd} | Mean |
| LD-Tyr TFA salt | 1.0E−05M | 11 | 32.5 | 22 | 64 | 72 | 68 |
| LD-Asp TFA salt | | 47.8 | 34.1 | 41 | 55 | 64 | 60 |
| LD-Glu TFA salt | | 36.6 | 49.4 | 43 | 110 | 115 | 112 |
| LD-LA2 TFA salt | | 18.7 | | 18.7 | 39 | 46 | 43 |
| LD-Lys TFA salt | | 0.3 | 18.7 | 10 | 79 | 63 | 71 |
| LD-Arg TFA salt | | 91.5 | 91.2 | 91 | 79 | 79 | 79 |
| LD (control) | | 30.4 | 20.2 | 25 | 59 | 78 | 68 |

### Experimental Example 8

### In-Vitro Absorption (Using Caco-2 Cells)

### Overview

P-glycoprotein (Pgp), Breast Cancer Resistance Protein (BCRP) and Multidrug Resistance-Associated Protein 2 (MRP2) are ATP-binding Cassette (ABC) transporter proteins located in the intestine and blood-brain barrier, among other tissues. Compounds that are substrates of these efflux pumps may be secreted back into the lumen of the intestine, resulting in poor absorption and bioavailability. Additionally, drugs that are targeted to the central nervous system but are Pgp or BCRP substrates, may be excluded from the brain, thus resulting in poor brain penetration.

### Cell Model

Caco-2 cells are human intestinal epithelial cells derived from a colorectal adenocarcinoma. This cell line has endogenously high expression of Pgp, BCRP and MRP2 and can be used as an in vitro model to assess compounds as substrates for these transporters

### Experimental Protocol

The assays are performed in both the apical to basolateral (A-B) and the B-A direction. The test compound is prepared at 10 μM in HBSS-HEPES (pH 7.4) with a final DMSO concentration of 1%. The working solution is centrifuged, and the supernatant is added to the donor side. The assay plate is incubated at 37° C. with gentle shaking for 60 min or 40 min for the A-B or B-A assay, respectively. For Pgp substrate assessment, the assays are run with and without 100 μM verapamil on both the A and B sides. For BCRP substrate assessment, the assays are run with and without 10 μM Ko143 on both the A and B sides. For MRP2 substrate assessment, the assays are run with and without 100 μM MK571 on both the A and B sides. Samples are aliquoted from the donor side at time zero and the end point, and from the receiver side at the end point.

### Reference Compounds

Propranolol (highly permeable), labetalol (moderately permeable), ranitidine (poorly permeable), and colchicine (P-glycoprotein substrate), estrone-3-sulfate (BCRP substrate), or CDCF (MRP2 substrate) are included in each assay.

### Analytical Method

Samples are analyzed by HPLC-MS/MS using selected reaction monitoring. The HPLC system consists of a binary LC pump with an autosampler, a C-18 column, and a gradient. Conditions may be adjusted when necessary.

### Cell Monolayer Integrity Marker

Fluorescein permeability is assessed in the A-B direction at pH 7.4 on both sides after the permeability assay with the test compound. The cell monolayer with a fluorescein permeability of less than 1.5×10−6 cm/s is considered intact.

### Data Analysis

The apparent permeability coefficient (Papp) of the test compound and its recovery are calculated as follows:

$$\begin{matrix}{P}_{app} ( cm / s ) = \frac{{V}_{R} \times {C}_{R , end}}{Δ t} \times \frac{1}{A \times ( {C}_{D , mid} - {C}_{R , mid} )} \\ Recovery ( \% ) = \frac{{V}_{D} \times {C}_{D , end} + {V}_{R} \times {C}_{R , end}}{{V}_{D} \times {C}_{D 0}} \times 100\end{matrix}$$

A is the surface area of the cell monolayer (0.11 cm²).
C is concentration of the test compound, expressed as peak area.
D denotes donor and R is receiver.
0, mid, and end denote time zero, mid-point, and end of the incubation.
Δt is the incubation time.
V is the volume of the donor or receiver.

### Experimental Example 8.1—A-B Permeability

The permeability capabilities of the several LDAA compounds, in their TFA salt form, were determined using the Caco-2 A-B method. The test was performed with and without verapamil, which is a permeation inhibitor, and the results are presented in Tables 22 (without verapamil) and 34 (with verapamil).

TABLE 22
Permeability of the compounds through the Caco-2 (A-B) (10⁻⁶ cm/sec)

| | Test | Permeability (10 ⁻⁶ cm/s) | | | | Percent Recovery(%) | | |
|---|---|---|---|---|---|---|---|---|
| Compound | Concentration | 1^{st} | 2^{nd} | Mean | Flags | 1^{st} | 2^{nd} | Mean |
| LD-Gly TFA salt | 1.0E−05M | 0.03 | 0.02 | <0 | BLQ* | 65 | 62 | 64 |
| LD-Tyr TFA salt | | 0.05 | 0.04 | <0 | BLQ* | 45 | 39 | 42 |
| LD-Trp TFA salt | | 0.04 | 0.03 | <0 | BLQ* | 52 | 51 | 51 |
| LD-Asp TFA salt | | | | | ND | | | |
| LD-Glu TFA salt | | | | | ND | | | |
| LD-LA1 TFA salt | | 0.2 | 0.21 | 0.2 | | 82 | 77 | 79 |
| LD-LA2 TFA salt | | 20.2 | 17.8 | 19 | | 86 | 91 | 89 |
| LD-Asn TFA salt | | 0.6 | 0.47 | 0.5 | | 62 | 78 | 70 |
| LD-Lys TFA salt | | 0.12 | 0.09 | <0.1 | BLQ* | 21 | 38 | 30 |
| LD-Gln TFA salt | | 1.83 | 1.47 | 1.7 | | 68 | 70 | 69 |
| LD-Arg TFA salt | | 0.1 | 0.09 | <0.1 | BLQ* | 26 | 26 | 26 |
| LD (control) | | 1.15 | 1.25 | 1.2 | | 37 | 22 | 29 |

*BLQ = below limit quantification

TABLE 23
Permeability of the compounds through the Caco-2 (A-B)
(10⁻⁶ cm/sec) in the presence of verapamil

| | Test | Permeability (10 ⁻⁶ cm/s) | | | | Percent Recovery(%) | | |
|---|---|---|---|---|---|---|---|---|
| Compound | Concentration | 1^{st} | 2^{nd} | Mean | Flags | 1^{st} | 2^{nd} | Mean |
| LD-Gly TFA salt | 1.0E−05M | 0.02 | 0.02 | <0.02 | BLQ* | 60 | 59 | 59 |
| LD-Tyr TFA salt | | 0.03 | 0.03 | <0.03 | BLQ* | 40 | 39 | 40 |
| LD-Trp TFA salt | | 0.03 | 0.02 | <0.03 | BLQ* | 50 | 45 | 47 |
| LD-Asp TFA salt | | | | | ND | | | |
| LD-Glu TFA salt | | | | | ND | | | |
| LD-LA1 TFA salt | | 0.04 | 0.04 | <0.04 | BLQ* | 58 | 67 | 62 |
| LD-LA2 TFA salt | | 26.42 | 20.96 | 23.7 | | 89 | 87 | 88 |
| LD-Asn TFA salt | | 0.64 | 0.52 | 0.6 | | 65 | 62 | 63 |
| LD-Lys TFA salt | | 0.1 | 0.09 | <0.1 | BLQ* | 33 | 33 | 33 |
| LD-Gln TFA salt | | 3.23 | 2.45 | 2.8 | | 74 | 69 | 71 |
| LD-Arg TFA salt | | 0.08 | 0.07 | <0.1 | BLQ* | 25 | 26 | 25 |
| LD (control) | | 0.5 | 0.56 | 0.5 | | 63 | 41 | 52 |

As shown in Tables 22 and 23, the LD-LA 2 TFA salt presented the highest mean permeability both with and without verapamil.

### Experimental Example 8.1—B-A Permeability

The permeability capabilities of the several LDAA compounds, in their TFA salt form, were determined using the Caco-2 B-A method. The test was performed with and without verapamil, and the results are presented in Tables 24 (without verapamil) and 25 (with verapamil).

TABLE 24
Permeability of the compounds through the Caco-2 (B-A) (10⁻⁶ cm/sec)

| | Test | Permeability (10 ⁻⁶ cm/s) | | | | Percent Recovery(%) | | |
|---|---|---|---|---|---|---|---|---|
| Compound | Concentration | 1^{st} | 2^{nd} | Mean | Flags | 1^{st} | 2^{nd} | Mean |
| LD-Gly TFA salt | 1.0E−05M | 0.38 | 0.33 | 0.4 | | 93 | 78 | 85 |
| LD-Tyr TFA salt | | 0.01 | 0.01 | <0.01 | BLQ* | 82 | 97 | 89 |
| LD-Trp TFA salt | | 0.01 | 0.01 | <0.01 | BLQ* | 71 | 82 | 77 |
| LD-Asp TFA salt | | | | | ND | | | |
| LD-Glu TFA salt | | | | | ND | | | |
| LD-LA1 TFA salt | | 0.28 | 0.22 | 0.3 | | 80 | 86 | 83 |
| LD-LA2 TFA salt | | 13.03 | 13.64 | 13.3 | | 79 | 69 | 74 |
| LD-Asn TFA salt | | 0.42 | 0.39 | 0.4 | | 85 | 84 | 85 |
| LD-Lys TFA salt | | 0.03 | 0.03 | <0.03 | BLQ* | 79 | 86 | 82 |
| LD-Gln TFA salt | | 1.01 | 0.79 | 0.9 | | 81 | 92 | 87 |
| LD-Arg TFA salt | | 0.03 | 0.02 | <0.03 | BLQ* | 82 | 89 | 85 |
| LD (control) | | 0.38 | 0.41 | <0.4 | BLQ* | 101 | 92 | 96 |

TABLE 25
Permeability of the compounds through the Caco-2 (B-A)
(10⁻⁶ cm/sec) in the presence of verapamil

| | Test | Permeability (10 ⁻⁶ cm/s) | | | | Percent Recovery(%) | | |
|---|---|---|---|---|---|---|---|---|
| Compound | Concentration | 1^{st} | 2^{nd} | Mean | Flags | 1^{st} | 2^{nd} | Mean |
| LD-Gly TFA salt | 1.0E−05M | 0.17 | 0.14 | 0.2 | | 89 | 97 | 93 |
| LD-Tyr TFA salt | | 0.01 | 0.01 | <0.01 | BLQ* | 81 | 85 | 83 |
| LD-Trp TFA salt | | 0.01 | 0.01 | <0.01 | BLQ* | 83 | 84 | 83 |
| LD-Asp TFA salt | | | | | ND | | | |
| LD-Glu TFA salt | | | | | ND | | | |
| LD-LA1 TFA salt | | 0.02 | 0.02 | <0.02 | BLQ* | 82 | 89 | 86 |
| LD-LA2 TFA salt | | 14.17 | 10.66 | 12.4 | | 84 | 98 | 91 |
| LD-Asn TFA salt | | 0.29 | 0.31 | 0.3 | | 85 | 89 | 87 |
| LD-Lys TFA salt | | 0.03 | 0.03 | <0.03 | BLQ* | 78 | 90 | 84 |
| LD-Gln TFA salt | | 1.35 | 1.04 | 1.2 | | 86 | 93 | 90 |
| LD-Arg TFA salt | | 0.02 | 0.02 | <0.02 | BLQ* | 82 | 88 | 85 |
| LD (control) | | 0.19 | 0.11 | <0.1 | BLQ* | 77 | 81 | 79 |

As shown in Tables 24 and 25, and similarly to the results presented in Tables 22 and 23, the LD-LA 2 TFA salt presented the highest mean permeability both with and without verapamil.

### Experimental Example 9—In Vivo Studies

### Example 9.1—Subcutaneous Bolus Treatment

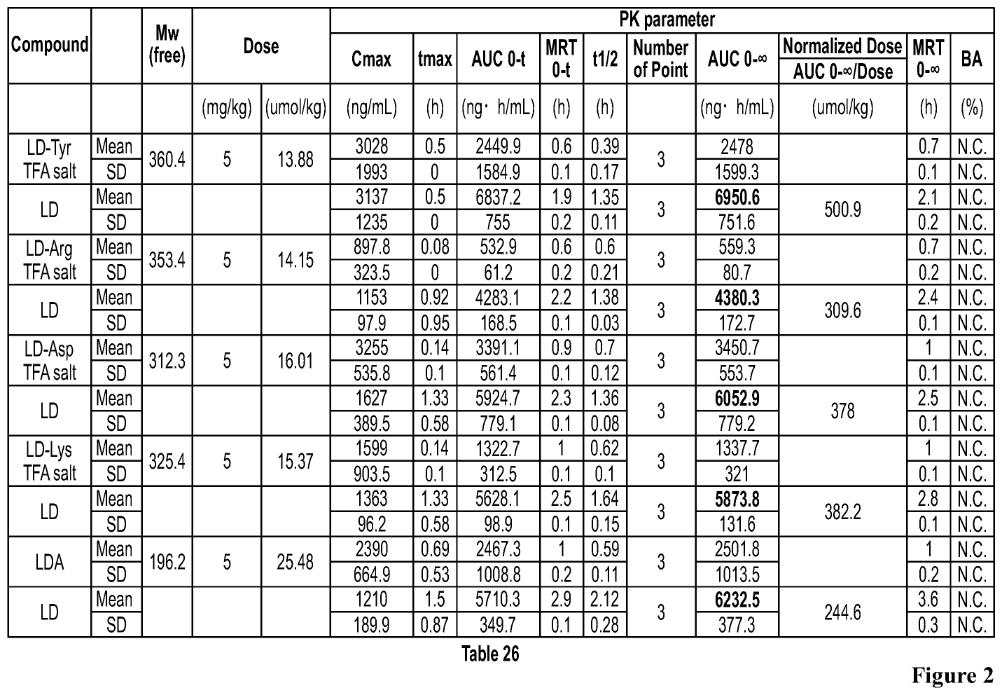

Several compounds (5 mg/Kg) were delivered to minipigs subcutaneously by bolus in order to examine the pharmacokinetic profile of those compounds and to compare them to one another. The compounds examined were LD-Tyr TFA salt, LD-Arg TFA salt, LD-Asp TFA salt, LD-Lys TFA salt and LDA (Dopamide). The bolus dose further comprised 1.25 mg/Kg carbidopa, 0.2% Tween® 80, 20 mM phosphate buffer and 137 mM NaCl, wherein the administered solution was prepared within an hour prior to administration. There were three repeats of each measurement. The pharmacokinetic parameters examined are described in FIGS. 2, 3 and 4.

FIG. 2 presents Table 26, which includes the pharmacokinetic parameters derived from the subcutaneous minipig bolus study. The tested compounds, as well as their levodopa metabolite, were examined and the amounts thereof determined. The examined parameters include Cₘₐₓ, tₘₐₓ, AUC₀₋ₜ, MRT₀₋ₜ, t_{1/2}, AUC_{0-∞}, normalized dose, MRT_{0-∞} and BA.

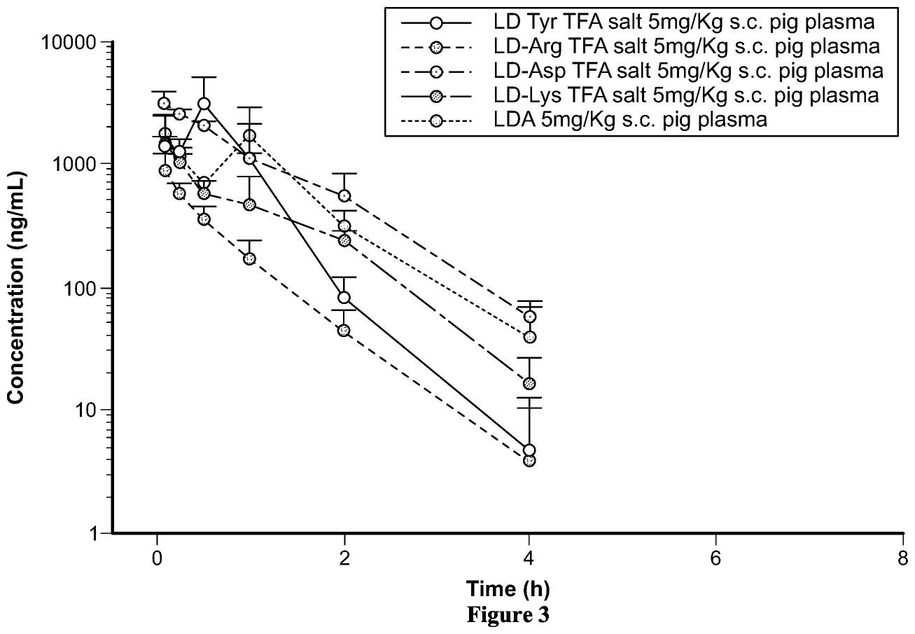

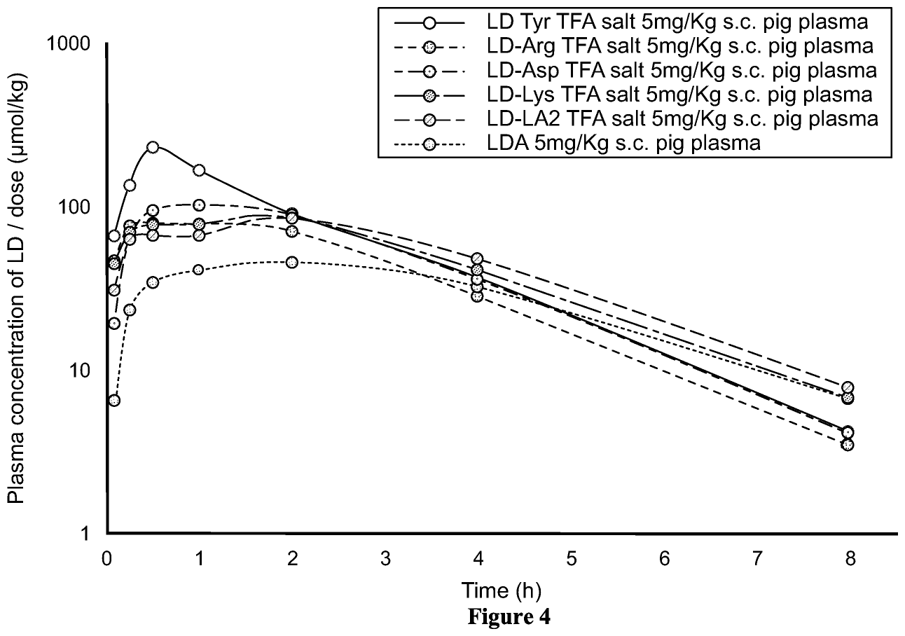

FIG. 3 is a graph presenting the LDAA compound concentration as a factor of time, following the subcutaneous bolus administration of 5 mg/Kg of each tested LDAA compound to minipigs. FIG. 4 is a graph presenting the levodopa concentration as a factor of time, following the subcutaneous bolus administration of 5 mg/Kg of each tested LDAA compound to minipigs. In this respect it is noted that levodopa is a metabolite of the LDAA compounds and therefore, the administration of the LDAA compounds provides a levodopa in the blood. It is further noted that, due to high speed centrifuge, the pharmacokinetic tests presented herein involve measurements performed in whole blood, not plasma.

### Example 9.2—24-Hour Subcutaneous Continuous Treatment—12.5% LD-Tyr Free Base Formulation

An LD-Tyr free base (12.5%) solution was applied to Göttingen minipigs continuously by an infusion pump for a period of 24 hours. The applied solution further comprised 0.75% CD, 25% NMP, 0.15% Na Bis, 0.1% NaOH, 0.3% Tween® 80 and WFI to complete to 100%. This formulation is related to herein as the 12.5% LD-Tyr formulation.

### Pharmacokinetic Studies

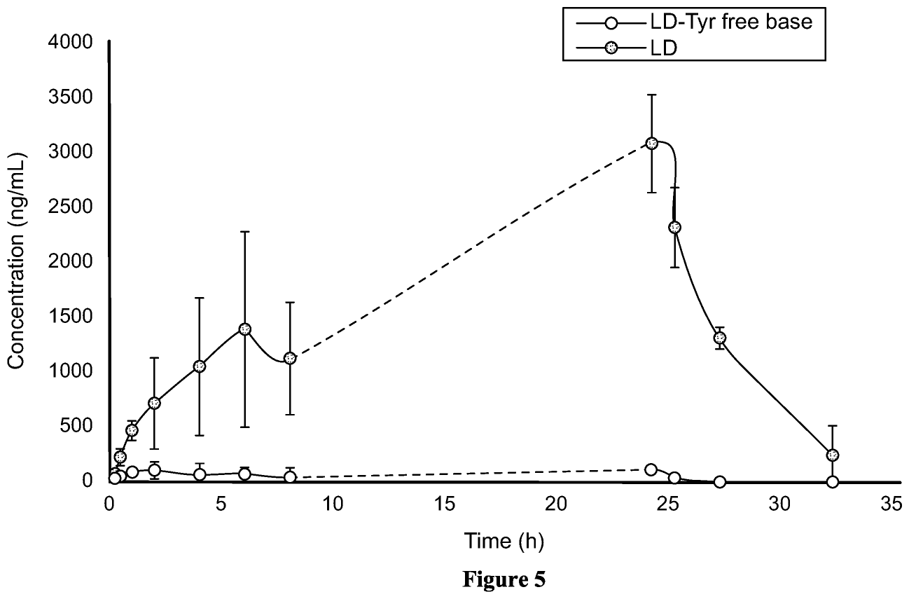

Sampling timepoints started at t=0 and terminated at t=32 after administration of the 12.5% LD-Tyr formulation. The pharmacokinetic results are described in Table 27 below and in FIG. 5, wherein the concentrations of both the tested compound, i.e., LD-Tyr free base, and its metabolite, levodopa, were measured at all time points.

TABLE 27

| | | LD-Tyr free base | | LD | |
|---|---|---|---|---|---|
| Treat- | Time | Concentration | | Concentration | |
| ment | (hrs) | Mean (ng/ml) | S.D. | Mean (ng/ml) | S.D. |
| LD- | 0 | 0 | | 0 | |
| Tyrfree | 0.25 | 37.34 | 42.78 | 85.07 | 73.76 |
| base | 0.5 | 46.99 | 34.60 | 233.44 | 90.78 |
| 12.5% | 1 | 84.92 | 78.45 | 474.97 | 416.48 |
| | 2 | 107.50 | 103.01 | 720.40 | 624.52 |
| | 4 | 66.09 | 50.65 | 1062.30 | 895.16 |
| | 6 | 79.16 | 84.37 | 1388.60 | 515.93 |
| | 8 | 44.88 | 37.54 | 1127.70 | 445.24 |
| | 24 | 115.72 | 54.40 | 3096.30 | 366.74 |
| | 25 | 38.44 | 21.01 | 2328.30 | 100.96 |
| | 27 | 6.28 | 5.83 | 1313.30 | 263.92 |
| | 32 | 0 | | 254.53 | 99.50 |

### Local Toxicity Studies

Initial data from local toxicity studies performed in Göttingen minipigs with the administration of the 12.5% LD-Tyr formulation (24 hours continuous subcutaneous treatment) provide an acceptable safety and local tolerability profile, i.e., a profile with no systemic or local drug related adverse reactions, such as cutaneous ulcers.

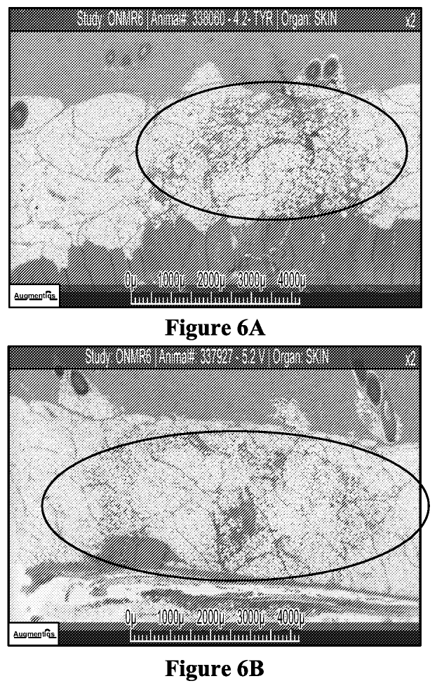

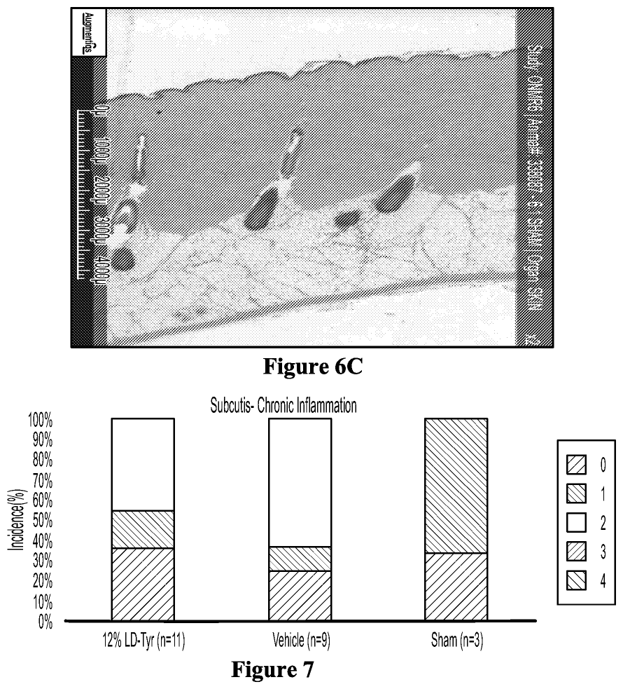

FIGS. 6A, 6B, 6C and 7 partially depict the data obtained from the 24 hour continuous administration Göttingen minipig study. Particularly, FIG. 6A presents a histopath obtained from the Göttingen minipigs after two weeks recovery from a 24 hour continuous subcutaneous administration of the LD-Tyr free base solution described above, FIG. 6B presents a histopath obtained from the Göttingen minipigs after two weeks recovery from a 24 hour continuous subcutaneous administration of the vehicle of the same solution, i.e., the solution without the LD-Tyr free base itself, and FIG. 6C presents a histopath obtained after 24 hours of having a sham (needle alone) inserted into the Göttingen minipigs. When reviewing those figures, and comparing them to one another, it appears that, while there are some artifacts of a minimal/mild chronic inflammation in FIGS. 6A and 6B (see particularly encircled areas in FIGS. 6A and 6B), the severity thereof is very low, thus showing the non-toxicity of the administered solution.

Further, when particularly comparing FIGS. 6A and 6B to one another, it appears that the severity of inflammation is very similar and therefore, it may be concluded that the vehicle itself causes most of the inflammation, not the LD-Tyr free base active ingredient.

Finally, FIG. 7 presents the % of incidence of inflammation and the severity thereof, wherein 0 is the lowest severity and 4 is the highest. As shown in FIG. 7, only relatively low severity inflammation incidents are present (0, 1 and 2, not 3 or 4) and further, the incidents are similar when administering the LD-Tyr free base solution and when administering the vehicle alone, i.e., the same solution without the LD-Tyr free base. It may therefore again be concluded that the vehicle itself causes most of the inflammation, not the LD-Tyr free base active ingredient.

### Experimental Example 10—Evaluation of In Vitro Conversion Efficiency Using Human Hepatocytes

Conversion efficiency from a prodrug to L-DOPA was evaluated with a metabolic test using human hepatocytes. A prodrug was incubated with human hepatocytes at 37° C. for 4 hours. A part of the reaction solution was sampled at each predetermined time and mixed with an organic solvent to stop the reaction. The reaction-stopped solution was centrifuged, and the obtained supernatant was measured with LC-MS/MS. The conversion efficiency to L-DOPA was evaluated as an amount of L-DOPA produced 4 hours after the start of the reaction. Table 28 shows the L-DOPA production amounts of the compounds of some of the examples of the present invention.

TABLE 28

| Example | L-DOPA production amount (nmol/L) |
|---|---|
| 4 | 652 |
| 5 | 704 |
| 6 | 785 |
| 8 | 558 |
| 13 | 932 |
| 18 | 729 |
| 19 | 1267 |
| 20 | 1313 |
| 21 | 718 |

As shown in the results of the above tests, it was confirmed that all compounds produced L-DOPA. From these results, efficient L-DOPA production in vivo is expected, and it is considered to be particularly useful as a therapeutic medicament for Parkinson's disease.

### Experimental Example 11—Comparative Solubility and Formulation Studies—11 LDAA Molecules

### Example 11.1—Comparative Solubility Tests

Table 29 lists 11 LDAA TFA salts, comprising one equivalent of TFA, which were prepared according to Example 1.

TABLE 29

| | | | | | % |
|---|---|---|---|---|---|
| | | LDAA | | | LDAA |
| | | TFA | | | TFA |
| | LDAA | salt | | | salt |
| | Mole- | mole- | | | equiv- |
| | cular | cular | | | alent |
| | weight | weight | | | to 30% |
| | (gr/ | (gr/ | LDAA | TFA | LDAA |
| LDAA | mol) | mol) | % | % | base |
| LD-Gly | 254.24 | 328.29 | 77.44 | 22.56 | 38.74 |
| LD-Tyr | 360.37 | 434.42 | 75.96 | 17.05 | 39.49 |
| LD-Trp | 383.4 | 457.45 | 77.08 | 16.19 | 38.92 |
| LD-Asp | 312.28 | 386.33 | 73.25 | 19.17 | 40.95 |
| LD-Glu | 326.31 | 400.36 | 74.11 | 18.50 | 40.48 |
| LD-LA 1 | 387.4 | 461.45 | 77.26 | 16.05 | 38.83 |
| LD-LA 2 | 387.4 | 461.45 | 77.26 | 16.05 | 38.83 |
| LD-Asn | 311.29 | 385.34 | 73.19 | 19.22 | 40.99 |
| LD-Lys | 325.37 | 399.42 | 74.05 | 18.54 | 40.51 |
| LD-Gln | 325.32 | 399.37 | 74.05 | 18.54 | 40.51 |
| LD-Arg | 353.38 | 427.43 | 75.61 | 17.32 | 39.68 |

A solution, as detailed in Table 30 was prepared:

TABLE 30

| | Final concentration |
|---|---|
| Ingredient | (% w/v) |
| Tween80 | 0.30 |
| Ascorbic acid | 0.50 |
| NAC | 0.50 |
| L-Arginine | 5.50 |
| Tromethamine (TRIS) | 11.50 |
| Water q.s.to | 100.00 |

Eleven formulations, each comprising one of the LDAA TFA, in an amount equivalent to 30% w/v of the corresponding LDAA base, and 70% w/v the stock solution, as detailed in Table 30, were prepared. Surprisingly, not all LDAAs were dissolved; rather, as detailed in Table 31, six of the eleven were dissolved, while five were not (wherein, the five that were not dissolved either did not dissolve during preparation, or demonstrated precipitation within an hour of the preparation of the formulations).

TABLE 31

| Dissolved | Not dissolved |
|---|---|
| LD-Tyr | LD-Gly |
| LD-Trp | LD-Glu |
| LD-Asp | LD-LA 2 |
| LD-LA 1 | LD-Asn |
| LD-Lys | LD-Gln |
| LD-Arg | |

### Experimental Example 11.2—Comparative Formulation Tests

The following formulations were prepared using the six LDAAs (in the TFA salt form) that demonstrated solubility. It is noted that the formulations were similarly to those above; however, CD was added, and further, the amounts of the antioxidants, i.e., ascorbic acid and NAC, were added at three different concentrations, as detailed in Tables 32a-c below. Further, an additional amount of arginine was added to the solution in order to adjust the pH to a physiologically acceptable pH.

TABLE 32a
Formulations comprising~0.1% w/v ascorbic acid and NAC

| | NB 144-15 | NB 144-15 | NB 144-15 | NB 144-15 | NB 144-15 | NB 144-15 |
|---|---|---|---|---|---|---|
| | (F-1) | (F-2) | (F-3) | (F-4) | (F-5) | (F-6) |
| Ingredient (% w/v) | LD-Tyr | LD-Trp | LD-Asp | LD-LA 1 | LD-Lys | LD-Arg |
| LDAA TFA | 35.43 | 33.83 | 33.32 | 33.34 | 37.46 | 37.49 |
| salt | | | | | | |
| LDAA base | 26.92 | 26.08 | 24.41 | 25.76 | 27.74 | 28.34 |
| equivalent | | | | | | |
| Carbidopa | 0.75 | 0.72 | 0.76 | 0.71 | 1.09 | 0.88 |
| monohydrate | | | | | | |
| Ascorbic acid | 0.09 | 0.09 | 0.08 | 0.09 | 0.09 | 0.09 |
| NAC | 0.09 | 0.09 | 0.08 | 0.09 | 0.09 | 0.09 |
| L-Arginine | 5.08 | 4.98 | 4.65 | 4.91 | 5.09 | 5.19 |
| Tromethamine | 10.62 | 10.41 | 9.73 | 10.27 | 10.65 | 10.85 |
| (TRIS) | | | | | | |
| Additional L- | 4.33 | 2.85 | 13.92 | 8.95 | 0.00 | 0.00 |
| Arginine (pH | | | | | | |
| adjustment) | | | | | | |
| Total L- | 9.41 | 7.83 | 18.58 | 13.86 | 5.09 | 5.19 |
| Arginine | | | | | | |
| pH measured | 7.62 | 7.48 | 7.23 | 6.99 | 7.09 | 7.36 |

TABLE 32b
Formulations comprising~0.8% w/v ascorbic acid and NAC

| | NB 144-18 | NB 144-18 | NB 144-18 | NB 144-18 | NB 144-18 | NB 144-18 |
|---|---|---|---|---|---|---|
| | (F-7) | (F-8) | (F-9) | (F-10) | (F-11) | (F-12) |
| Ingredient (% w/v) | LD-Tyr | LD-Trp | LD-Asp | LD-LA 1 | LD-Lys | LD-Arg |
| LDAA TFA salt | 32.09 | 33.25 | 33.47 | 32.22 | 36.69 | 34.59 |
| LDAA base | 24.38 | 25.63 | 24.51 | 24.90 | 27.17 | 26.16 |
| equivalent | | | | | | |
| Carbidopa (on dry | 0.67 | 0.75 | 0.70 | 0.68 | 0.77 | 0.74 |
| basis) | | | | | | |
| Ascorbic acid | 0.81 | 0.86 | 0.82 | 0.83 | 0.89 | 0.87 |
| NAC | 0.81 | 0.86 | 0.82 | 0.83 | 0.89 | 0.87 |
| L-Arginine | 4.44 | 4.71 | 4.51 | 4.58 | 4.91 | 4.81 |
| Tromethamine | 9.27 | 9.85 | 9.43 | 9.58 | 10.27 | 10.05 |
| (TRIS) | | | | | | |
| Additional L- | 9.80 | 5.30 | 19.03 | 12.29 | 1.85 | 1.79 |
| Arginine (pH | | | | | | |
| adjustment) | | | | | | |
| Total L-Arginine | 14.24 | 10.01 | 23.54 | 16.88 | 6.76 | 6.60 |
| pH measured | 8.06 | 7.67 | 7.76 | 7.43 | 7.37 | 7.58 |

TABLE 32c
Formulations comprising~0.4% w/v ascorbic acid and NAC

| | NB 144-20 | NB 144-20 | NB 144-20 | NB 144-20 | NB 144-20 | NB 144-20 |
|---|---|---|---|---|---|---|
| | (F-13) | (F-14) | (F-15) | (F-16) | (F-17) | (F-18) |
| Ingredient (% w/v) | LD-Tyr | LD-Trp | LD-Asp | LD-LA 1 | LD-Lys | LD-Arg |
| LDAA TFA salt | 31.67 | 32.94 | 32.50 | 32.87 | 35.81 | 36.37 |
| LDAA base | 24.06 | 25.39 | 23.81 | 25.40 | 26.52 | 27.50 |
| equivalent | | | | | | |
| Carbidopa (on dry | 0.67 | 0.74 | 0.69 | 0.71 | 0.71 | 0.76 |
| basis) | | | | | | |
| Ascorbic acid | 0.40 | 0.42 | 0.40 | 0.41 | 0.44 | 0.45 |
| NAC | 0.40 | 0.42 | 0.40 | 0.41 | 0.44 | 0.45 |
| L-Arginine | 4.44 | 4.66 | 4.37 | 4.55 | 4.82 | 5.00 |
| Tromethamine | 9.27 | 9.75 | 9.13 | 9.52 | 10.09 | 10.45 |
| (TRIS) | | | | | | |
| Additional L- | 8.20 | 4.80 | 17.50 | 11.31 | 0.87 | 0.92 |
| Arginine (pH | | | | | | |
| adjustment) | | | | | | |
| Total L-Arginine | 12.64 | 9.46 | 21.86 | 15.86 | 5.69 | 5.92 |
| pH measured | 8.04 | 7.74 | 7.69 | 7.32 | 7.36 | 7.50 |

The formulations prepared as detailed in Tables 32a, 32b and 32c were (a) held at room temperature for two days; (b) transferred to a refrigerator (2-8° C.) for two days; and (c) transferred from the refrigerator to room temperature for an additional two days, during which they were assessed again for precipitants. The formulations were then returned to the refrigerator (2-8° C.) and assessed for precipitants on day 40. The physical stability of each formulation was assessed on the first two days and again on the last two days. The physical stability of the formulations was assessed visually. A clear solution was considered to be stable, while a solution comprising precipitants was considered to be physically unstable. The stability results of the formulations of Tables 32a, 32b and 32c are detailed in Tables 33a, 33b and 33c, respectively.

TABLE 33a

| | | Day 1 | Day 2 | Day 5 | Day 6 | Day 40 |
|---|---|---|---|---|---|---|
| NB 144-15 | LD-Tyr | Stable | Stable | Precipitated | Precipitated | Precipitated |
| ~0.1% Asc + | LD-Trp | Stable | Stable | Stable | Stable | Stable |
| ~0.1% NAC | LD-Asp | Stable | Stable | Stable | Stable | Stable |
| | LD-LA 1 | Stable | Stable | Stable | Stable | Stable |
| | LD-Lys | Precipitated | Precipitated | Precipitated | Precipitated | Precipitated |
| | LD-Arg | Stable | Stable | Stable | Stable | Stable |

TABLE 33b

| | | Day 1 | Day 2 | Day 5 | Day 6 | Day 40 |
|---|---|---|---|---|---|---|
| NB 144-20 | LD-Tyr | Stable | Stable | Stable | Stable | Stable |
| ~0.8% Asc + | LD-Trp | Stable | Stable | Stable | Stable | Stable |
| ~0.8% NAC | LD-Asp | Stable | Stable | Stable | Stable | Stable |
| | LD-LA 1 | Stable | Stable | Stable | Stable | Stable |
| | LD-Lys | Stable | Stable | Precipitated | Precipitated | Precipitated |
| | LD-Arg | Stable | Stable | Stable | Stable | Stable |

TABLE 33c

| | | Day 1 | Day 2 | Day 5 | Day 6 | Day 40 |
|---|---|---|---|---|---|---|
| NB 144-18 | LD-Tyr | Stable | Stable | Stable | Stable | Stable |
| ~0.4% Asc + | LD-Trp | Stable | Stable | Stable | Stable | Stable |
| ~0.4% NAC | LD-Asp | Stable | Stable | Stable | Stable | Stable |
| | LD-LA 1 | Precipitated | Precipitated | Precipitated | Precipitated | Precipitated |
| | LD-Lys | Stable | Stable | Precipitated | Precipitated | Precipitated |
| | LD-Arg | Stable | Stable | Stable | Stable | Stable |

As presented in Tables 33a-c, the physical stability of the prepared formulations is dependent on the LDAA used, as well as on the amount of the antioxidant in the solution. For instance, the LD-LA 1 solution, which was found to be physically stable over the course of 40 days when 0.1% and 0.4% ascorbic acid and NAC were used, was not stable when 0.9% of each of ascorbic acid and NAC were used. It is noted that almost all formulations are stable for at least 48 hours at room temperature. It is possible that if after the first 48 hours the formulations remained only at 2-8° C. they would have remained stable for the entirety of the 40 test days, or even longer. It is also possible that if the formulations were placed at 2-8° C. immediately after being prepared, they would have remained stable for the entirety of the 40 test days, or even longer.

### Experimental Example 12—LD-Arg, LD-Lys and LD-Tyr Formulations

Formulations as described in the tables below were prepared by adding a CD solution and dissolving all ingredients together. The CD for the LD-Arg formulation was prepared as follows: WFI was added to a bottle, after which Tween® 80 and sodium bisulfite were added, stirred to dissolution, and heated to 60° C. CD was added, stirred for 1-2 minutes to achieve homogenization. Finally, L-Arginine was added, the bottle was flushed with nitrogen, tightly closed and stirred 15 min. Dissolution was verified and the preparation was allowed to cool to ambient temperature. The pH was measured, and the preparation was transferred to a measurement bottle, where the volume was completed to the predefined final volume by adding WFI. The preparation was then filtered through sterile 0.22 μm nylon filter, transferred to 20 ml vials, after which nitrogen was purged into the headspace and the vials were frozen at −20° C. until use.

The CD solution for the LD-Tyr and LD-Lys formulations was prepared as follows: WFI was added to a bottle. Tween® 80 and sodium bisulfite were added, stirred to dissolution and heated to 60° C. CD was added, and stirred for 1-2 minutes to achieve homogenization. NaOH was added, after which the bottle was washed with nitrogen, closed tightly and stirred for 15 minutes. Dissolution was verified and the preparation was allowed to cool to ambient temperature. The pH was measured, and preparation was transferred to a measurement bottle, in which the volume was completed to final predefined volume by adding WFI. The preparation was then filtered through a sterile 0.22 μm nylon filter, transferred to 20 ml vials, after which nitrogen was purged into headspace and the vials were frozen at −20° C. until use.

TABLE 34

| Ingredient | LD-Tyr/ | LD-Arg/ | LD-Lys/ |
|---|---|---|---|
| (% w/v) | CD | CD | CD |
| LD-Tyr base | 12 | 0 | 0 |
| LD-Arg HCl | 0 | 17.5 | 0 |
| (base equivalent) | | | |
| LD-Lys TFA | 0 | 0 | 12.5 |
| (base equivalent) | | | |
| Carbidopa (on | 0.75 | 0.75 | 0.75 |
| dry basis) | | | |
| N-MP | 24.7 | 0 | 0 |
| Tween ® 80 | 0.3 | 0.3 | 0.3 |
| L-Arginine | 0 | 5.4 | 0 |
| Sodium hydroxide | 0.2 | 0 | 2.54 |
| Sodium bisulfite | 0.15 | 0.15 | 0.15 |
| WFI q.s to | 100 | 100 | 100 |
| PH | 6.56 | 7.24 | 7.60 |

TABLE 35

| Ingredient | LD-Arg/ | LD-Arg/ | LD-Arg/ |
|---|---|---|---|
| (% w/v) | CD | CD | CD |
| LD-Arg HCl | 11 | 20.5 | 28 |
| (free base equivalent) | | | |
| Carbidopa (on dry | 0.75 | 0.75 | 0.75 |
| basis) | | | |
| Na Bisulfite | 0.15 | 0.15 | 0.15 |
| Sodium hydroxide | 0.85 | 1.62 | 2.42 |
| Tween ® 80 | 0.30 | 0.30 | 0.30 |
| WFI q.s. to | 100 | 100 | 100 |
| pH final | 7.02 | 7.05 | 7.22 |

TABLE 36

| Ingredient | LD-Tyr/ | LD-Lys/ | LD-Lys/ | LD-Arg/ |
|---|---|---|---|---|
| (% w/v) | CD | CD | CD | CD |
| LD-Tyr base | 13.7 | 0 | 0 | 0 |
| LD-Lys HCl | 0 | 15.0 | 22.0 | 0 |
| (base equivalent) | | | | |
| LD-Arg HCl | 0 | 0 | 0 | 30.0 |
| (base equivalent) | | | | |
| Carbidopa (on | 0.75 | 0.75 | 0.75 | 0.75 |
| dry basis) | | | | |
| N-MP | 24.70 | 0 | 0 | 0 |
| Tween ® 80 | 0.30 | 0.30 | 0.30 | 0.30 |
| Ascorbic acid | 0.20 | 0.20 | 0.20 | 0.20 |
| NAC | 0.20 | 0 | 0 | 0 |
| L-Cysteine HCl | 0 | 0.25 | 0.25 | 0.25 |
| NaOH | 0.96 | 2.88 | 4.20 | 5.60 |
| WFI q.s. to | 100 | 100 | 100 | 100 |
| Final pH | 7.50 | 6.90 | 6.90 | 7.40 |

TABLE 37

| Ingredient | LD-Tyr/ | LD-Lys/ | LD-Lys/ | LD-Arg/ | LD-Arg/ |
|---|---|---|---|---|---|
| (% w/v) | CD | CD | CD | CD | CD |
| LD-Tyr base | 13.5 | 0 | 0 | 0 | 0 |
| LD-Lys HCl | 0 | 15.0 | 22.0 | 0 | 0 |
| (base equivalent) | | | | | |
| LD-Arg HCl | 0 | 0 | 0 | 16 | 23.5 |
| (base equivalent) | | | | | |
| Carbidopa (on | 0.75 | 0.75 | 0.75 | 0.75 | 0.75 |
| dry basis) | | | | | |
| N-MP | 24.70 | 0 | 0 | 0 | 0 |
| Tween ® 80 | 0.30 | 0.30 | 0.30 | 0.30 | 0.30 |
| Ascorbic acid | 0.20 | 0.20 | 0.20 | 0.20 | 0.20 |
| NAC | 0.20 | 0 | 0 | 0 | 0 |
| L-Cysteine HCl | 0 | 0.25 | 0.25 | 0.25 | 0.25 |
| NaOH | 0.90 | 2.90 | 4.20 | 1.4 | 2.05 |
| WFI q.s. to | 100 | 100 | 100 | 100 | 100 |
| Final pH | 7.5 | 6.90 | 6.90 | 7.37 | 7.35 |

TABLE 38

| Ingredient | LD-Tyr/ | LD-Tyr/ | LD-Lys/ | LD-Arg/ |
|---|---|---|---|---|
| (% w/v) | CD | CD | CD | CD |
| LD-Tyr base | 12.9 | 20.0 | 0 | 0 |
| LD-Lys HCl | 0 | 0 | 23 | 0 |
| base equivalent | | | | |
| LD-Arg HCl | 0 | 0 | 0 | 26.0 |
| base equivalent | | | | |
| Carbidopa (on | 0.75 | 0.75 | 0.75 | 0.75 |
| dry basis) | | | | |
| N-MP | 24.70 | 0 | 0 | 0 |
| Tween ®-80 | 0.30 | 0.30 | 0.30 | 0.30 |
| Ascorbic acid | 0.20 | 0.50 | 0.20 | 0.20 |
| NAC | 0.20 | 0.50 | 0 | 0 |
| L-Cysteine HCl | 0 | 0 | 0.25 | 0.25 |
| NaOH | 1.01 | 1.10 | 3.80 | 2.33 |
| L-Arginine | 0 | 10.00 | 0 | 0 |
| WFI q.s. to | 100 | 100 | 100 | 100 |
| Final pH | 7.61 | 8.6 | 6.82 | 7.24 |

TABLE 39
high LD-Tyr concentration formulations

| Ingredient (% w/v) | F-1 | F-2 | F-3 | F-4 | F-5 | F-6 |
|---|---|---|---|---|---|---|
| LD-Tyr | 25.00 | 30.00 | 30.00 | 37.00 | 44.00 | 44.00 |
| Carbidopa (on dry | 0.75 | 0.75 | 0.75 | 0.75 | 0.75 | 0.75 |
| basis) | | | | | | |
| L-Arginine | 4.20 | 5.00 | 18.10 | 6.00 | 8.00 | 25.70 |
| Tromethamine | 8.70 | 10.50 | 0 | 13.00 | 15.00 | 0 |
| (TRIS) | | | | | | |
| Ascorbic acid | 0.50 | 0.50 | 0.50 | 0.50 | 0.50 | 0.50 |
| NAC | 0.50 | 0.50 | 0.50 | 0.50 | 0.50 | 0.50 |
| Tween ® 80 | 0.30 | 0.30 | 0.30 | 0.30 | 0.30 | 0.30 |
| Water q.s. to | 100.0 | 100.0 | 100.0 | 100.0 | 100.0 | 100.0 |
| pH | 8.42 | 8.25 | 8.2 | 8.3 | 8.2 | 8.08 |

TABLE 40
physical stability data for formulations presented in
Table 39 above and additional formulations

| APIs | | | | | | | | | |
|---|---|---|---|---|---|---|---|---|---|
| LD- | | | | | | | | Refrigerated | |
| Tyr | CD | Counter ions* | | | | 25° C. | | (2-8° C.) | |
| (%) | (%) | L-Arginine | NaOH | TRIS | pH | t = days | Appearance | t = days | Appearance |
| 30.0 | 0.75 | 18.10 | 0 | 0 | 8.36 | — | Not tested | 50 | Stable |
| 30.0 | 0.75 | 7.00 | 0 | 10.5 | 8.23 | — | Not tested | 44 | precipitated |
| 38.0 | 0 | 21.90 | 0 | 0 | 8.43 | 70 | Stable | — | Not tested |
| 37.0 | 0.75 | 6.00 | 0 | 12.124 | 8.12 | 26 | Stable | 26 | Stable |
| 37.0 | 0.75 | 6.00 | 0 | 13 | 8.14 | 12 | Stable | 12 | Stable |
| 44.0 | 0.75 | 25.72 | 0 | 0 | 8.43 | 27 | Stable | 70 | Stable |
| 44.0 | 0 | 21.4 | 0.60 | 0 | 8.29 | 1 | Stable | — | Not tested |
| 44.0 | 0.75 | 21.4 | 0.60 | 0 | 8.29 | 12 | Stable | — | Not tested |
| 44.0 | 0.75 | 21.86 | 0.60 | 0 | 8.57 | 24 | Stable | 56 | Stable |
| 44.0 | 0.75 | 6.27 | 0 | 12.69 | 8.02 | 3 | Stable | 1 | Stable |
| 44.0 | 0.75 | 6.76 | 0 | 13.726 | 8.08 | 26 | Stable | 26 | Stable |
| 44.0 | 0.75 | 8.00 | 0 | 15 | 8.15 | 14 | Stable | 7 | Stable |
| 44.0 | 0.75 | 25.70 | 0 | 0 | 8.34 | 14 | Stable | 14 | Stable |

*it is noted that certain materials may be referred to herein as a base, counterion, or solvent; however, the definitions for those materials are all equivalent

TABLE 41
LD-Tyr Concentration and Arginine:Tris
Ratio Effect on Stability

| LD-Tyr | Arg | TRIS | | Molar | Physical |
|---|---|---|---|---|---|
| (%) | (%) | (%) | pH | ratio | Stability* |
| 20 | 0 | 8.8 | 7.94 | 1:1.20 | Precipitated |
| | | | | | overnight |
| 30 | 4.5 | 9.72 | 8.00 | 1:1.20 | Stable |
| | | | | | overnight |
| 44 | 6.8 | 13.8 | 8.02 | 1:1.20 | Stable |
| | | | | | overnight |
| 37 | 5.8 | 11.7 | 8.12 | 1:1.32 | Stable |
| | | | | | overnight |
| 44 | 7.33 | 14.9 | 8.08 | 1:1.30 | Stable |
| | | | | | overnight |

*The formulations were stored at room temperature overnight, after which the physical stability was assessed

### Example 53—LD-Tyr Formulations with Varying Concentrations of CD, TRIS and L-Arginine

The following formulations were prepared according to the procedures detailed in Example 12.

TABLE 42

| Ingredient | NB 144-4 | NB 130-145 | NB 130-145 | NB 130-148 | NB144-32 | NB144-34 | NB 144-39 |
|---|---|---|---|---|---|---|---|
| (% w/v) | 30% | (F1) 30% | (F2) 37% | (F3) 44% | (F1) | (F2) | 30% (F3) |
| LD-Tyrosine | 30.00 | 30.00 | 37.00 | 44.00 | 37.00 | 44.00 | 30.00 |
| (Nominal) | | | | | | | |
| Carbidopa | 0.75 | 1.00 | 1.00 | 1.00 | 0.50 | 0.50 | 0.50 |
| (on dry basis) | | | | | | | |
| Ascorbic acid | 0.50 | 0.50 | 0.50 | 0.50 | 0.50 | 0.50 | 0.50 |
| NAC | 0.50 | 0.50 | 0.50 | 0.50 | 0.50 | 0.50 | 0.50 |
| L-Arginine | 5.50 | 5.50 | 6.50 | 8.00 | 6.50 | 8.00 | 5.50 |
| Tromethamine | 11.50 | 11.50 | 13.00 | 15.00 | 13.00 | 15.00 | 11.50 |
| (TRIS) | | | | | | | |
| Tween80 | 0.30 | 0.30 | 0.30 | 0.30 | 0.30 | 0.30 | 0.30 |
| WFI q.s. to | 100.0 | 100.0 | 100.0 | 100.0 | 100.0 | 100.0 | 100.0 |
| Final pH | 8.19 | 8.17 | 8.20 | 8.14 | 8.13 | 8.17 | 8.25 |
| Molar | 1.446 | 1.446 | 1.349 | 1.340 | 1.349 | 1.340 | 1.446 |
| ratio (Bases/ | | | | | | | |
| L-Tyrosine) | | | | | | | |

TABLE 43
NB 144-4 Formulation Analytical Stability Results

| Storage temp. 2-8° C. | | | | % retention | |
|---|---|---|---|---|---|
| | t = 0 | t = 1 week | t = 2 weeks | t = 1 week | t = 2 weeks |
| LD- | 327.05 | 323.22 | 326.20 | 98.8 | 99.7 |
| Tyr | | | | | |
| CD | 7.37 | 7.36 | 7.30 | 99.9 | 99.1 |
| Storage temp. 25° C. | | | | % retention | |
| | t = 0 | t = 1 week | t = 2 weeks | t = 1 week | t = 2 weeks |
| LD- | 327.05 | 330.81 | 318.15 | 101.1 | 97.3 |
| Tyr | | | | | |
| CD | 7.37 | 7.32 | 7.41 | 99.3 | 100.5 |
| Storage temp. 40° C. | | | | % retention | |
| | t = 0 | t = 1 week | t = 2 weeks | t = 1 week | t = 2 weeks |
| LD- | 327.05 | 304.24 | NA | 93.0 | NA |
| Tyr | | | | | |
| CD | 7.37 | 7.26 | NA | 98.5 | NA |

As shown in Table 43, the LD-Tyr and CD in the F53-1 formulation are highly stable, even when stored at temperatures up to 40° C.

TABLE 44
Stability Results for NB130-145 (F1), NB130-145(F2) and NB130-148 (F3)

| | t = 0 | | | | t = 28 h at 32° C. | | | |
|---|---|---|---|---|---|---|---|---|
| | LD-Tyr | | CD | | LD-Tyr | | CD | |
| Batch No | mg/mL | % | mg/mL | % | mg/mL | % | mg/mL | % |
| NB130- | 319.65 | 106.55 | 9.83 | 98.33 | 318.17 | 106.06 | 9.73 | 97.26 |
| 145 (F1) | | | | | | | | |
| NB130- | 397.37 | 107.40 | 9.87 | 98.67 | 396.01 | 107.03 | 9.78 | 97.82 |
| 145 (F2) | | | | | | | | |
| NB130- | 464.11 | 105.48 | 9.68 | 96.78 | 451.69 | 102.63 | 9.57 | 95.70 |
| 148 (F3) | | | | | | | | |

*it is noted that all % in Table 44 are compared to the measured concentrations of 30% LD-Tyr and 1% CD

As shown in Table 44, when formulations NB130-145 (F1), NB130-145 (F2) and NB130-148 (F3) are stored at 32° C. for 28 h, the concentrations of the active ingredients, as measured by HPLC, hardly change, i.e., those formulations are stable for at least 28 h at 32° C.

TABLE 45a
NB 144-32 (F1)-37% LD-Tyr/0.5% CD

| | t = 0 | t = 28 h at 32° C. | % Recovery |
|---|---|---|---|
| LD-Tyr | 364.15 | 352.22 | 96.73 |
| CD | 4.78 | 4.77 | 99.90 |

TABLE 45b
NB 144-34 (F2)-44% LD-Tyr/0.5% CD

| | t = 0 | t = 28 h at 32° C. | % Recovery |
|---|---|---|---|
| LD-Tyr | 437.98 | 424.09 | 96.83 |
| CD | 4.84 | 4.78 | 98.86 |

TABLE 45c
NB 144-39 (F3)-30% LD-Tyr/0.5% CD

| | t = 0 | t = 28 h at 32° C. | % Recovery |
|---|---|---|---|
| LD-Tyr | 295.42 | 291.94 | 98.82% |
| CD | 4.86 | 4.74 | 97.53% |

As shown in Tables 45a, 45b and 45c, when formulations NB144-32 (F1), NB144-34 (F2), NB144-39 (F3) are stored at 32° C. for 28 h, the concentrations of the active ingredients, as measured by HPLC, remain above 96%, and even above 98%, i.e., those formulations are stable for at least 28 h at 32° C. It is noted that the recovery % compared the amount of any given material as measured at t=28 h (or any other presented value) compared to the amount of that material, at measured at t=0.

### EQUIVALENTS

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention. All numbers expressing quantities of ingredients, reaction conditions, and so forth used in the specification are to be understood as being modified in all instances by the term “about”, even if the term “about” is not specifically recited in respect to any of the disclosed embodiments.

### INCORPORATION BY REFERENCE

The entire contents of all patents, published patent applications, websites, and other references referred to herein, are hereby expressly incorporated herein in their entireties by reference.

1. A liquid pharmaceutical composition comprising:
a levodopa amino acid conjugate (LDAA) of the general formula (I): 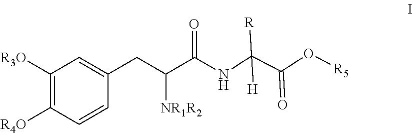
an enantiomer, diastereomer, racemate, ion, zwitterion, pharmaceutically acceptable salt thereof, or any combination thereof, wherein:
R is an amino acid side chain;
R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;
R₃ and R₄ are each independently selected from the group consisting of H, (C₁-C₃)alkyl, C₃-C₆cycloalkyl, phenyl, and —P(═O)(OR′)₂;
R₅ is selected from the group consisting of H, (C₁-C₃)alkyl, C₃-C₆cycloalkyl and phenyl;
R′ is independently selected, for each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and
R″ is independently selected, for each occurrence, from the group consisting of a (C₁-C₆)alkyl, (C₂-C₆)alkenyl, and (C₂-C₆)alkynyl; and
a pharmaceutically acceptable excipient.

2. The liquid pharmaceutical composition according to claim 1, wherein R is an amino acid side chain selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, and lanthionine side chains.

3. The liquid pharmaceutical composition according to claim 1 or 2, wherein R is an amino acid side chain selected from the group consisting of arginine, tyrosine, lysine, tryptophan, aspartic acid, or lanthionine 1.

4. The liquid pharmaceutical composition according to any one of claims 1-3, wherein the LDAA is represented by:

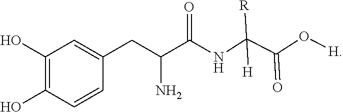

5. The liquid pharmaceutical composition according to any one of claims 1-4 comprising one LDAA conjugate, or a mixture of two or more different LDAA conjugates, each represented by Formula I, or an enantiomer, diastereomer, racemate, ion, zwitterion, pharmaceutically acceptable salt thereof, or any combination thereof.

6. The liquid pharmaceutical composition of any one of claims 1-5, comprising between about 10 to about 45% w/v of one or more of the LDAA conjugate represented by Formula I.

7. The liquid pharmaceutical composition according to any one of claims 1-6, wherein the liquid pharmaceutical composition has a pH in the range of between about 5 to about 10 at about 25° C.

8. The liquid pharmaceutical composition according to any one of claims 1-7, further comprising a decarboxylase inhibitor.

9. The liquid pharmaceutical composition according to claim 8, wherein the decarboxylase inhibitor is carbidopa.

10. The liquid pharmaceutical composition according to any one of claims 1-9, further comprising a base.

11. The liquid pharmaceutical composition according to claim 10, wherein the base is selected from the group consisting of arginine, NaOH, tris(hydroxymethyl)aminomethane (TRIS), and any combination thereof.

12. The liquid pharmaceutical composition according to any one of claims 10-11, wherein said liquid pharmaceutical composition comprises between about 0.1% to about 30% w/v of the base.

13. The liquid pharmaceutical composition according to any one of claims 8-12, wherein said liquid pharmaceutical composition comprises between about 0.25 to about 1.5% w/v of the decarboxylase inhibitor.

14. The liquid pharmaceutical composition according to any one of claims 1-13, further comprising an antioxidant or a combination of two or more antioxidants.

15. The liquid pharmaceutical composition according to claim 14, wherein the antioxidant is each independently selected from the group consisting of ascorbic acid or a salt thereof, a cysteine, a bisulfite or a salt thereof, glutathione, a tyrosinase inhibitor, a Cu²⁺ chelator, and any combination thereof.

16. The liquid pharmaceutical composition according to claim 14 or 15, wherein said liquid pharmaceutical composition comprises between about 0.05 to about 1.5% w/v of the antioxidant or the combination of antioxidants.

17. The liquid pharmaceutical composition according to any one of claims 1-16, further comprising at least one of: a catechol-O-methyltransferase (COMT) inhibitor, a monoamine oxidase (MAO) inhibitor, a surfactant, a buffer, an acid, a solvent, and any combination thereof.

18. The liquid pharmaceutical composition according to claim 17, wherein the buffer is TRIS.

19. The liquid pharmaceutical composition according to any one of claims 17-18, wherein said liquid pharmaceutical composition comprises between about 5.0 to about 40.0% w/v of the buffer.

20. A method of treating neurodegenerative conditions and/or conditions characterized by reduced levels of dopamine in the brain, wherein the method comprises administering an effective amount of a liquid pharmaceutical composition to a patient in need thereof, wherein the liquid pharmaceutical composition comprises a levodopa amino acid conjugate (LDAA) of the general formula (I): 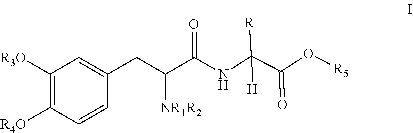
an enantiomer, diastereomer, racemate, ion, zwitterion, pharmaceutically acceptable salt thereof, or any combination thereof, wherein
R is an amino acid side chain;
R₁ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;
R₃ and R₄ are each independently selected from the group consisting of H, (C₁-C₃)alkyl, C₃-C₆cycloalkyl, phenyl, and —P(═O)(OR′)₂;
R₅ is selected from the group consisting of H, (C₁-C₃)alkyl, C₃-C₆cycloalkyl and phenyl;
R′ is independently selected, for each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and
R″ is independently selected, for each occurrence, from the group consisting of a (C₁-C₆)alkyl, (C₂-C₆)alkenyl, and (C₂-C₆)alkynyl; and
a pharmaceutically acceptable excipient.

21. The method according to claim 20, wherein the neurodegenerative condition is Parkinson's disease.

22. The method according to claim 20 or claim 21, wherein the liquid pharmaceutical composition is administered concomitantly to the patient with an additional active ingredient.

23. The method according to claim 22, wherein the additional active ingredient is selected from the group consisting of a decarboxylase inhibitor, a COMT inhibitor, a MAO inhibitor, and any combination thereof.

24. The method according to any one of claims 20-23, wherein the liquid pharmaceutical composition is administered substantially continuously to the patient.

25. The method according to any one of claims 20-24, wherein the liquid pharmaceutical composition is administered subcutaneously.

26. A levodopa amino acid conjugate (LDAA) of the general formula (III): 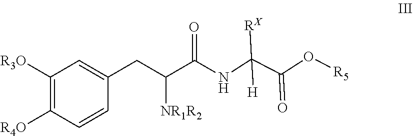
an enantiomer, diastereomer, racemate, ion, zwitterion, pharmaceutically acceptable salt thereof, or any combination thereof, wherein
R^{X} is an amino acid side chain; or an O-phosphorylated amino acid side chain thereof;
R¹ and R₂ are each independently selected from the group consisting of H, (C₁-C₆)alkyl, (C₂-C₆)alkenyl, (C₂-C₆)alkynyl, C₃-C₆cycloalkyl, phenyl, —O—C(═O)—R′, —C(═O)—OR′, —C(═O)—R′, —C(═S)—R′, —O—C(═O)—NR′R′, —O—C(═S)—NR′R′, and —O—C(═O)—R″;
R₃ and R₄ are each independently selected from the group consisting of H, (C₁-C₃)alkyl, C₃-C₆cycloalkyl, phenyl, and —P(═O)(OR′)₂;
R₅ is selected from the group consisting of H, (C₁-C₃)alkyl, C₃-C₆cycloalkyl and phenyl;
R′ is independently selected, in each occurrence, from the group consisting of H, (C₁-C₆)alkyl, (C₂-C₆)alkenyl, C₃-C₆cycloalkyl, phenyl, and heteroaryl bonded to the nitrogen through a ring carbon; and
R″ is independently selected, in each occurrence, from the group consisting of a (C₁-C₆)alkyl, (C₂-C₆)alkenyl, and (C₂-C₆)alkynyl.

27. The levodopa amino acid conjugate (LDAA) according to claim 26, wherein the amino acid side chain in R^{x} is selected from the group consisting of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, ornithine, lanthionine and 3,4-dihydroxyphenylalanine side chain.

28. The levodopa amino acid conjugate (LDAA) according to any one of claims 25 to 27, wherein the amino acid side chain in R^{x} is selected from the group consisting of arginine, lysine, serine, glycine, alanine, valine, phenylalanine, tyrosine, ornithine, and 3,4-dihydroxyphenylalanine.

29. The levodopa amino acid conjugate (LDAA) according to any one of claims 25 to 28, wherein each one of R₁, R₂ and R₅ are H; R₃, and R₄ independently is H or —P(═O)(OR′)₂; and R′ is H.

30. The levodopa amino acid conjugate (LDAA) selected from the group consisting of:
(2S)-2-amino-3-(3-hydroxy-4-phosphonooxyphenyl)propionamide,
2-[[(2S)-2-amino-3-(3-hydroxy-4-phosphonooxyphenyl)propanoyl]amino]ethanesulfonic acid,
(2S)-2-amino-6-[[(2S)-2-amino-3-(3-hydroxy-4-phosphonooxyphenyl)propanoyl]amino]hexanoic acid, and
(2S)-2-amino-5-[[(2S)-2-amino-3-(3-hydroxy-4-phosphonooxyphenyl)propanoyl]amino]pentanoic acid.

31. A method of treating Parkinson's disease in a patient in need thereof, comprising subcutaneously administering to the patient an effective amount of a compound of claim 30.
